(19) United States
(12) Patent Application Publication
(10) Pub. No.: US 2010/0074083 A1
(43) Pub. Date: Mar. 25, 2010

(54) RESIN COMPOSITION AND MOLDED PRODUCT OBTAINED BY MOLDING THE RESIN COMPOSITION

(75) Inventors: Atsushi Shibuya, Chiba (JP); Yukihiro Kumamoto, Chiba (JP); Masaru Wada, Fukuoka (JP); Shota Abe, Chiba (JP); Yuji Terado, Hiroshima (JP); Naruyoshi Mita, Chiba (JP); Kaori Matoishi, Chiba (JP)

Correspondence Address: BUCHANAN, INGERSOLL & ROONEY PC, POST OFFICE BOX 1404, ALEXANDRIA, VA, 22313-1404, US

(73) Assignee: MITSUI CHEMICALS, INC., Minato-ku, Tokyo (JP)

(21) Appl. No.: 12/443,374

(22) PCT Filed: Oct. 11, 2007
(86) PCT No.: PCT/JP2007/001102

§ 371 Date: Mar. 27, 2009

(30) Foreign Application Priority Data
Oct. 17, 2006 (JP) 2006-283105

(51) Int. Cl. G11B 7/00; C08K 5/3492; C07D 403/14

(52) U.S. Cl. 369/100; 524/100; 544/198; G9B/7

(57) ABSTRACT

Provided are a resin composition comprising 100 parts by mass of the polymer having an alicyclic structure at least in a part of a repeating structural unit and 0.05 to 5 parts by mass of a hindered amine compound having a carbon atom at a ratio of from 67% by weight to 80% by weight in the molecular structure and having a molecular weight of from 500 to 3500, a novel piperidine derivative having a piperidylaminotriazine skeleton, a molded product such as an optical component obtained by molding the resin composition, and an optical pickup device which employs the optical component.

### TECHNICAL FIELD

The present invention relates to a resin composition providing excellent optical characteristics, a piperidine derivative having a piperidylaminotriazine skeleton, a molded product such as an optical component obtained by molding the resin composition and an optical pickup device employing the optical component.

### BACKGROUND ART

The optical pickup device (also referred to as an optical head, an optical head device, or the like) for replaying and recording the information on a light information recording medium (also referred to as an optical disk, or a medium) such as a CD (a compact disk), DVD (a digital video disk, or a digital versatile disk) have been developed and produced, and thus popularized. Recently, the standard of the information recording medium which enabled the higher density information recording has been researched and developed.

Such optical pickup device forms a spot by collecting beam of light emitted from mainly a laser diode as a light source through an optical system including an optical component such as a beam shaping prism, a collimator, a beam splitter, an objective lens, or the like, onto the information recording surface of an optical disc, next collects reflection from an information recording hole (also called as a pit) on the recording surface through a same optical system onto a sensor at this time, and then converts into an electrical signal, to playback the information. During this time, ‘0’ and ‘1’ information are discriminated based on the phenomenon that the light beam of reflection varies according to the shape of the information recording hole. On the information recording surface of an optical disc, a protective layer made of plastic, which is also called as a cover glass, is provided as a protective substrate.

When recording information on recording type media such as CD-R, CD-RW, and the like, a spot resulted from the laser beam is formed on a recording surface and a thermochemical change is generated in a recording material on the recording surface. Accordingly, for example in the case of CD-R, the thermal diffusive pigment is irreversibly changed and a shape same to the information recording hole is formed. In the case of CD-RW, since a phase change-type material is used, a reversible change between a crystalline state and non-crystalline state by the thermochemical change is generated, and thus the rewriting of the information is possible.

For the optical pickup device for playing back the information from an optical disc of a CD standard, the numerical aperture (NA) of an objective lens is around 0.45, and the wavelength of a light source for use is around 785 nm. In addition, as for the recording, ones having 0.50 in approximate is a lot used. Herein, the thickness of a protective substrate for an optical disc of the CD standard is 1.2 mm.

A CD has been widely popularized as an optical information recording medium, and for the last few years, a DVD is popularized. The DVD is increased in its information recording capacity by making the thickness of the protective substrate thinner than the CD and also by miniaturizing the information recording hole. While a recording capacity of a CD is about 600 to 700 MB (Mega Bite), a DVD has a large recording capacity of about 4.7 GB (Giga Byte), thus being used a lot as a distribution medium to which a moving image such as a movie picture is recorded.

In addition, the optical pickup device for playing back the information from an optical disc of a DVD standard is principally the same as that of the CD. However, since the information recording hole is miniaturized as described above, the optical pickup device employs an objective lens having the NA of around 0.60, and a light source having the wavelength of around 655 nm. Further, as for the recording, ones having 0.65 in approximate is more often used. Herein, the thickness of the protective substrate for an optical disc of the DVD standard is 0.6 mm.

A recording type for the optical disc of the DVD standard is already put to practical use, and there are various standards such as DVD-RAM, DVD-RW/R, DVD+RW/R, and the like. The technical principal of these optical disc is also same as that of the CD standard. As described above, there has been proposed an optical disc of high density/high capacity. This optical disc is to use the light source for providing the light having a wavelength of around 405 nm, which is the light source for providing so-called a blue-violet laser. For such ‘optical disc of high density/high capacity, even if the wavelength to be used is determined, the thickness of the protective substrate, recording capacity, NA, and the like cannot be determined without variation.

In order to improve the recording density substantially, it is necessary to reduce the thickness of the protective substrate of an optical disc and to increase the NA accordingly. Alternatively, the thickness of the protective substrate and NA can be in the same standard as the conventional optical disc standard. At this time, the physical recording density is not significantly increased, but the properties required as the optical system become relatively gradual.

In specific, there is proposed a protective substrate such as further reduced ones having the thickness of 0.1 mm, or ones same with DVD of 0.6 mm.

The optical component to be used in the above-described optical pickup device is mostly formed by an injection molding with a plastic resin or pressure molding with a glass. Of these, the glass-made optical component is generally small in the refractive-index variance to a temperature change. Therefore, this element can be used in a beam shaping prism disposed nearby a light source which is the heat source. However there is a problem that the manufacturing cost is high. Therefore, it is less employed in each of optical components of collimator, coupling lens, objective lens, and the like. On the other hand, the plastic resin-made optical component has a merit that the manufacturing cost is low as it is molded by injection, and thus is used a lot to a large extent. However, since the plastic material has an absorption in the wavelength area to various degrees or another, there is a problem that the optical properties for a use are deteriorated.

Further, in order to perform a playing back of information (reading) or recording of information at high speed, it is necessary to improve the light amount to surely form a spot of collected light. For this, a most simple method is to increase the light emitting amount of the diode by raising the power of a laser diode, but due to this if optical properties involved in the use are increasingly deteriorated, a problem arises in that the optical properties as designed cannot be attained. In addition, increase in an atmospheric temperature due to the raise of the laser power becomes a factor that promotes a deterioration of the resin. Further, if the operation is carried out at high speed, the actuator also operates at high speed, and thus generated heat also becomes a factor that promotes a deterioration of the resin.

Accordingly, there is proposed various efforts to control the change of the optical properties at the time of use.

For example, in Patent Document 1, there is disclosed a resin composition comprising 0.03 to 1 parts by mass of a hindered amine light-resistant stabilizer, 0.002 to 2 parts by mass of phenol antioxidant, and 0.002 to 1 parts by mass of phosphorous antioxidant, based on 100 parts by mass of a thermoplastic norbornene resin (for example, a hydrogenated product of ring-opening polymer of 1,4-methano-1,4,4a,9a-tetrahydrofluorene). However, the stability for light of a resin composition disclosed in Patent Document 1 is not sufficient, and thus is not appropriate to be used in an optical pickup device having the blue-violet laser light source. In addition, there is a flaw in transmittance that it is lowered due to a coloring as salt is formed from the phenol antioxidant and the hindered amine light-resistant stabilizer. There are also problems that the foaming at the time of molding is easy to occur, and since the birefringence is poor, an optical component of high density cannot be obtained.

Also, for example in Patent Document 2, there is disclosed a resin composition comprising a vinyl alicyclic hydrocarbon polymer and a hindered amine light-resistant stabilizer having the number average molecular weight (Mn) of 1,000 to 10,000. This resin composition is excellent in processing stability, and capable to obtain a molded product excellent in light-resistant stability, thermal resistance, and transparency. According to the method, the foaming at the time of molding and the birefringence are improved as compared to the above-described technique, but still the stability for light is insufficient, and thus is not appropriate to be used in an optical pickup device having the blue-violet laser light source. In addition, this method has a flaw in that white turbidity occurs due to the blue-violet laser light irradiation.

Further, in Patent Document 3, as the resin composition having excellent weather resistance, light resistance, transparency, thermal resistance, and a low dusting characteristic at the time of molding process, and exhibiting excellent optical properties when molded to an optical component, there is disclosed a weather-resistant resin characterized by containing (A) a cyclic polyolefin resin, (B) benzotriazole UV absorbent having the molecular weight of 300 or more, the vapor pressure at 20° C. temperature of 1×10⁻⁸ Pa or less, and the 5% weight reducing temperature with a heat loss measurement of 200° C. or above, and (C) a hindered amine light stabilizer having the molecular weight of 500 or more, the vapor pressure at 20° C. temperature of 1×10⁻⁶ Pa or less, and the 5% weight reducing temperature with a heat loss measurement of 250° C. or above. According to the method, the thermal resistance is improved and the foaming at the time of molding is controlled as in the above-described technique, but there is absorption with the benzotriazole ultraviolet absorber, and thus is not appropriate to be used in an optical pickup device having the blue-violet laser light source. In addition, there is a flaw that the water absorption is high.

In Patent Document 4, in order to obtain a molded product with no coloration and no color change although irradiated with UV ray for a long period, there is disclosed a technique of mixing pellet A formed from a resin composition containing 100 parts by mass of a vinyl alicyclic hydrocarbon polymer and 0.001 to 2.0 parts by mass of an antioxidant with pellet B formed from a resin composition containing 100 parts by mass of a vinyl alicyclic hydrocarbon polymer and 2 to 20 parts by mass of a light-resistant stabilizer at a ratio of 5≦A/B≦50 by mass, and then melt-molding the resultant. However, the stability at the time of molding is deteriorated, and the transparency of the resin and the stability for the light are both insufficient, thus is not appropriate to be practically used in an optical pickup device using the blue-violet laser light source. In addition, the method is inappropriate for a large-scale production as the manufacturing and molding processes are complicated.

In the Patent Document 5, there is disclosed a resin composition comprising a polymer (A) comprising an ethylenic unsaturated monomer unit which is produced by subjecting an aromatic vinyl monomer to an addition polymerization reaction, and then hydrogenating an aromatic ring, and an antioxidant (B) having a phosphate ester structure and a phenol structure in a molecule such as 6-[3-(3-t-butyl-4-hydroxy-5-methylphenyl)propoxy]-2,4,8,10-tetra kis-t-butyldibenzo[d,f][1.3.2]dioxaphosphepin. It is described that the molded product of the resin composition is excellent in mechanical strength, and is not colored even with the irradiation of a light beam such as a blue-violet laser with a short wavelength and a high strength. However, the optical properties are still not sufficiently stable due to a deterioration of the resin during the use. Thus, it is difficult that the resin composition is used for an optical pickup device using a blue-violet laser beam source.

Furthermore, outdoor components such as solar cells and sunshine roofs of automobiles and windows are used outdoors. For these outdoor components, glass and the like are used, but a molded product of a resin composition has become in use as it is easily reduced in weight and has excellent moldability. These outdoor components are exposed to a solar light, and thus it is required to have light resistance. However, the conventional outdoor components may be deteriorated in transparency due to the deterioration of the resin during use, and accordingly, it is difficult to use those at outdoor.

Also, in order to improve the weather resistance of the molded product comprised of the resin composition, a hindered amine light stabilizer is used as a light stabilizer (Patent Documents 6 to 8).

[Patent Document 1] JP-A-09-268250

[Patent Document 2] Pamphlet of International Patent Publication WO 01/092412

[Patent Document 3] JP-A-2001-72839

[Patent Document 4] JP-A-2003-276047

[Patent Document 5] JP-A-2004-83813

[Patent Document 6] JP-A-01-50858

[Patent Document 7] JP-A-61-238777

[Patent Document 8] JP-A-62-030757

### DISCLOSURE OF THE INVENTION

It is an object of the present invention to provide a resin composition which is capable of providing a molded product having excellent light resistance, transparency, and the like, suppressed deterioration of the optical characteristics when a blue-violet laser light source is used, a novel piperidine derivative having a piperidylaminotriazine skeleton, which is capable of giving excellent weather resistance to a molded product, a molded product such as an optical component obtained by molding the resin composition, and an optical pickup device that utilizes an optical component.

The present inventors have found that resin composition comprising a polymer having an alicyclic structure at least in a part of a repeating structural unit, and a specific hindered amine compound can solve the above problems, thereby completing the present invention.

Specifically, the resin composition of the present invention comprises 100 parts by mass of the polymer having an alicyclic structure at least in a part of a repeating structural unit and 0.05 to 5 parts by mass of a hindered amine compound having a carbon atom at a ratio of from 67% by weight to 80% by weight in the molecular structure and having a molecular weight of from 500 to 3500.

Further, the novel piperidine derivative of the present invention is represented by the following General Formula (20):

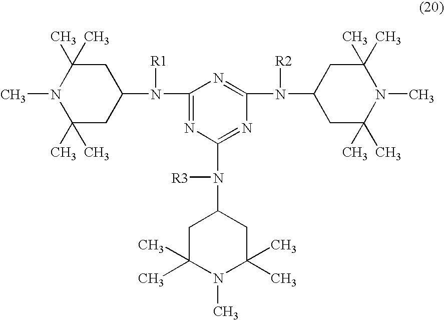

[wherein R1 to R3 may be the same as or different from each other, and each represent an alkyl group having 1 to 18 carbon atoms].

The piperidine derivative can be used as a hindered amine compound contained in the resin composition of the present invention.

Further, the present invention provides a molded product obtained by molding the resin composition.

Moreover, the present invention provides an optical pickup device which utilizes the molded product as an optical component.

According to the present invention, a resin composition which is capable of providing a molded product having excellent light resistance, transparency, and the like, suppressed deterioration of the optical characteristics when a blue-violet laser light source is used, a novel piperidine derivative which is capable of giving excellent weather resistance to a molded product, a molded product such as an optical component obtained by molding the resin composition, and an optical pickup device that utilizes an optical component can be obtained.

### BRIEF DESCRIPTION OF DRAWINGS

FIG. 1 is a drawing showing the optical pickup device according to the present invention.

### PREFERRED EMBODIMENTS FOR CARRYING OUT THE INVENTION

Hereinbelow, the present invention is described in detail.

[Polymer Having an Alicyclic Structure at Least in a Part of a Repeating Structural Unit]

The polymer having an alicyclic structure at least in a part of a repeating structural unit of the present invention (which may be hereinafter simply referred to the “polymer having an alicyclic structure”) may be any one having an alicyclic structure at least in a part of a repeating unit of the polymer, and specifically, it preferably includes a polymer having one or two or more kinds of the structures represented by General Formula (3):

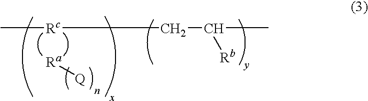

In Formula (3), x and y each represent a copolymerization ratio, and are each a real number satisfying 0/100≦y/x≦95/5. x and y are based on moles.

n represents a number of a substituent Q, and is a real number satisfying 0≦n≦2, and preferably 0.

R^{a} is a 2+n valent group selected from the group consisting of hydrocarbon groups having 2 to 20 carbon atoms, and preferably 2 to 12 carbon atoms.

R^{b} is a hydrogen atom, or a monovalent group selected from the group consisting of hydrocarbon groups having 1 to 10 carbon atoms.

R^{c} is a tetravalent group selected from the group consisting of hydrocarbon groups having 2 to 10 carbon atoms, and preferably 2 to 5 carbon atoms.

Q is COOR^{d}. R^{d} is a hydrogen atom, or a monovalent group selected from the group consisting of a hydrocarbon group having 1 to 10 carbon atoms. Preferably, R^{d} is a hydrogen atom, or a hydrocarbon group having 1 to 3 carbon atoms.

Furthermore, R^{a}, R^{b}, R^{c} and Q may be each one kind, or a combination of two or more kinds thereof at any ratio.

Further, in the above General Formula (3), R^{a} is preferably one, or two or more kinds of the divalent group selected from hydrocarbon groups having 2 to 12 carbon atoms, and more preferably, in a case of n=0, it is a divalent group represented by General Formula (7), and most preferably, it is a divalent group represented by the following General Formula (7) in which p is 0 or 1. The structure of R^{a} may be only one kind or two or more kinds may be used in combination.

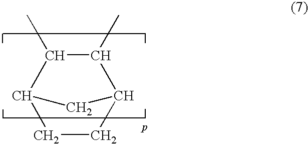

Here, in Formula (7), p is an integer of 0 to 2.

Further, in the above General Formula (3), examples of R^{b} include a hydrogen atom, a methyl group, an ethyl group, an n-propyl group, an i-propyl group, an n-butyl group, and a 2-methylpropyl group, preferably, a hydrogen atom and/or a methyl group, and most preferably, a hydrogen atom.

Further, in the above General Formula (3), examples of R^{c} include, in a case of n=0, those represented by the following General Formulae (8) to (10).

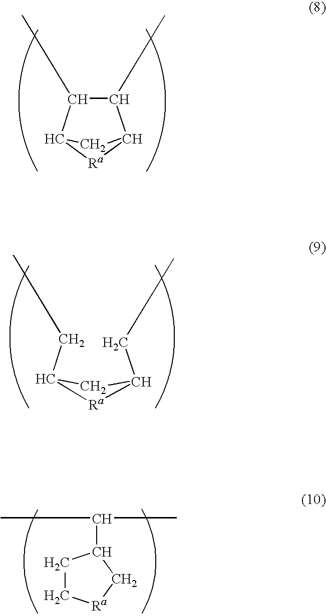

In Formulae (8) to (10), R^{a} indicates the same as in the above General Formula (3).

Further, in the above General Formula (3), n is preferably 0.

Further, the type of polymerization in the present invention is not limited at all, and well-known various types of polymerization such as addition polymerization, ring-opening polymerization, and the like can be applied. Examples of the addition polymerization include a random copolymer, a block copolymer, an alternating copolymer, and the like. In the present invention, a random copolymer is preferably used since it inhibits the deterioration of optical performances.

When a resin used as a main component has the above structure, a high-precision optical component having excellent optical properties such as transparency, refractive index, birefringent index, and the like, can be obtained.

(Examples of the Polymer Having an Alicyclic Structure at Least in a Part of a Repeating Structural Unit)

When the polymer represented by the above General Formula (3) is largely classified, the polymers are classified into the following (i) to (iv) categories:

(i) a copolymer of ethylene or α-olefin and cyclic olefin;

(ii) a ring-opening polymer or a hydrogenated product thereof;

(iii) a vinyl alicyclic hydrocarbon polymer; and

(iv) other polymers.

Hereinbelow, these are described orderly.

### ((i) Copolymer of Ethylene or α-Olefin and Cyclic Olefin)

(i) The copolymer of ethylene or α-olefin and cyclic olefin is a cyclic olefin copolymer represented by General Formula (4). For example, it comprises a structural unit (A) derived from ethylene or straight or branched α-olefin having 3 to 30 carbon atoms and structural unit (B) derived from a cyclic olefin.

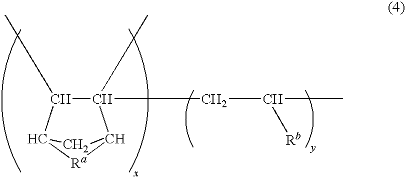

In Formula (4), R^{a} is a divalent group selected from the group consisting of hydrocarbon groups having 2 to 20 carbon atoms, and preferably 2 to 12 carbon atoms. R^{b} is a hydrogen atom or a monovalent group selected from the group consisting of hydrocarbon groups having 1 to 10 carbon atoms, and preferably 1 to 5 carbon atoms.

Further, R^{a} and R^{b} may be each one kind, or may have a combination of two or more kinds thereof at any ratio.

x and y each represent a copolymerization ratio, and are each a real number satisfying 5/95≦y/x≦95/5, preferably 50/50≦y/x≦95/5, and more preferably 55/45≦y/x≦80/20. x and y are based on moles.

(Structural Unit (A) Derived from Ethylene or α-Olefin)

The structural unit (A) derived from ethylene or α-olefin is a structural unit derived from ethylene or straight or branched α-olefin having 3 to 30 carbon atoms as follows.

Specific examples thereof include ethylene, propylene, 1-butene, 1-pentene, 1-hexene, 3-methyl-1-butene, 3-methyl-1-pentene, 3-ethyl-1-pentene, 4-methyl-1-pentene, 4-methyl-1-hexene, 4,4-dimethyl-1-hexene, 4,4-dimethyl-1-pentene, 4-ethyl-1-hexene, 3-ethyl-1-hexene, 1-octene, 1-decene, 1-dodecene, 1-tetradecene, 1-hexadecene, 1-octadecene, 1-eicosene, and the like. Among these, ethylene is preferable. These structural units derived from ethylene or α-olefin may be included in two or more kinds within the above range that the effect of the invention is not impaired.

(Structural Unit (B) Derived from a Cyclic Olefin)

The structural unit (B) derived from a cyclic olefin comprises at least one kind selected from the group consisting of structural units derived from a cyclic olefins represented by the following General Formula (11), General Formula (12), and General Formula (13).

The cyclic olefin represented by the following General Formula (11) has the following structure.

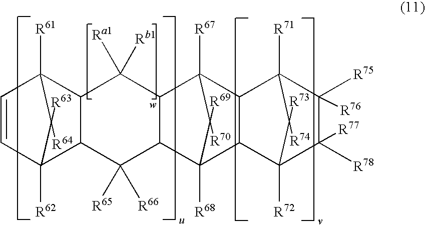

In Formula (11), u is 0 or 1; v is 0 or a positive integer; and w is 0 or 1. When w is 1, a ring represented by incorporating w is a 6-membered ring, and when w is 0, the ring is a 5-membered ring. R⁶¹ to R⁷⁸, and R^{a1} and R^{b1} may be the same as or different from each other, and is a hydrogen atom, a halogen atom, or a hydrocarbon group.

The halogen atom is a fluorine atom, a chlorine atom, a bromine atom, or an iodine atom. Examples of the hydrocarbon group generally include an alkyl group having 1 to 20 carbon atoms, an alkyl halide group having 1 to 20 carbon atoms, a cycloalkyl group having 3 to 15 carbon atoms, or an aromatic hydrocarbon group.

More specific examples of the alkyl group include methyl, ethyl, propyl, isopropyl, amyl, hexyl, octyl, decyl, dodecyl, and octadecyl, and the like. An example of the alkyl halide group includes a group in which the above alkyl group having 1 to 20 carbon atoms is substituted with one or more halogen atom(s). Examples of the cycloalkyl group include a cyclohexyl group, and the like. Examples of the aromatic hydrocarbon group include phenyl, naphthyl, and the like.

Further, in the above General Formula (11), R⁷⁵ and R⁷⁶, R⁷⁷ and R⁷⁸, R⁷⁵ and R⁷⁷, R⁷⁶ and R⁷⁸, R⁷⁵ and R⁷⁸, or R⁷⁶ and R⁷⁷ may be bonded or combined to each other to form a monocyclic or polycyclic group, and thus-formed monocyclic or polycyclic group may have a double bond. However, from the viewpoint of thermal resistance, the polycyclic is preferable to the monocyclic since a copolymer of high glass transition temperature (Tg) can be obtained with a smaller content of the polycyclic. Also, there is an advantage that the small amount of cyclic olefin is put for a production. Specific examples of the monocyclic or polycyclic group formed herein include the followings.

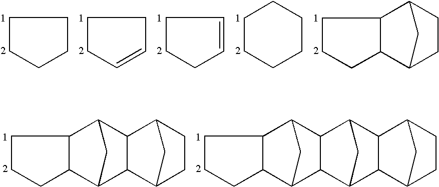

In the above examples, carbon atoms numbered 1 or 2 respectively represent a carbon atom to which R⁷⁵ (R⁷⁶) or R⁷⁷ (R⁷⁸) is bonded in the above General Formula (11).

An alkylidene group may be formed with R⁷⁵ and R⁷⁶ or R⁷⁷ and R⁷⁸. The alkylidene group usually has 2 to 20 carbon atoms, and specific examples thereof include ethylidene, propylidene, isopropylidene, and the like.

The cyclic olefin represented by General Formula (12) has the following structure.

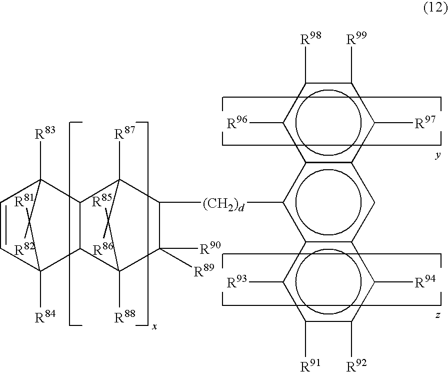

In Formula (12), x and d are each an integer of 0 or 1 or more; and y and z are each 0, 1, or 2. Further, R⁸¹ to R⁹⁹ may be the same as or different from each other, and are each a hydrogen atom, a halogen atom, an aliphatic hydrocarbon group, an aromatic hydrocarbon group, or an alkoxy group, a carbon atom to which R⁸⁹ and R⁹⁰ are bonded, and a carbon atom to which R⁹³ is bonded or a carbon atom to which R⁹¹ is bonded may be bonded directly or via an alkylene group having 1 to 3 carbon atoms, and when y=z=0, R⁹⁵ and R⁹², or R⁹⁵ and R⁹⁹ may be bonded to each other to form a monocyclic or polycyclic aromatic ring.

As the halogen atom, the same halogen atoms mentioned in the above Formula (11) can be exemplified.

The aliphatic hydrocarbon group is exemplified by an alkyl group having 1 to 20 carbon atoms, and a cycloalkyl group having 3 to 15 carbon atoms. More specific examples of the alkyl group include methyl, ethyl, propyl, isopropyl, amyl, hexyl, octyl, decyl, dodecyl, octadecyl, and the like. Examples of the cycloalkyl group include cyclohexyl, and the like.

Examples of the aromatic hydrocarbon group include an aryl group, an aralkyl group, and the like, and specifically phenyl, tolyl, naphthyl, benzyl, phenylethyl, and the like.

Examples of the alkoxy group include methoxy, ethoxy, propoxy, and the like. Herein, a carbon atom to which R⁸⁹ and R⁹⁰ are bonded, and a carbon atom to which R⁹³ is bonded or a carbon atom to which R⁹¹ is bonded may be bonded directly or via an alkylene group having 1 to 3 carbon atoms. That is, when the above two carbon atoms are bonded via an alkylene group, each of R⁸⁹ and R⁹³, or R⁹⁰ and R⁹¹ jointly forms any one alkylene group selected from a methylene group (—CH₂—), an ethylene group (—CH₂CH₂—), or a propylene group (—CH₂CH₂CH₂—).

Further, when y=z=0, R⁹⁵ and R⁹², or R⁹⁵ and R⁹⁹ may be bonded to each other to form a monocyclic or polycyclic aromatic ring. When y=z=0, specific examples of the aromatic ring formed with R⁹⁵ and R⁹² include the following aromatic rings. However, from the viewpoint of thermal resistance, the polycyclic is preferable to the monocyclic since a copolymer of high glass transition temperature (Tg) can be obtained with a smaller content of the polycyclic. Also, there is an advantage that the small amount of cyclic olefin can be put for production.

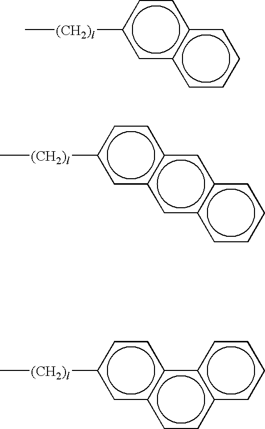

Herein, I is the same as d in the above General Formula (12).

The cyclic olefin represented by General Formula (13) has the following structure.

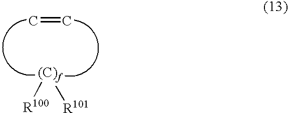

In Formula (13), R¹⁰⁰ and R¹⁰¹ may be the same as or different from each other, and each represent a hydrogen atom or a hydrocarbon group having 1 to 5 carbon atoms; and f satisfies Examples of the hydrocarbon group having 1 to 5 carbon atoms preferably include an alkyl group, an alkyl halide group, and a cycloalkyl group. Specific examples are as shown in the specific examples of R⁶¹ to R⁷⁸ in the above Formula (11).

Specific examples of the structural unit (B) derived from the cyclic olefin represented by the above General Formula (11), (12) or (13) include a bicyclo-2-heptene derivative (a bicyclohepto-2-ene derivative), a tricyclo-3-decene derivative, a tricyclo-3-undecene derivative, a tetracyclo-3-dodecene derivative, a pentacyclo-4-pentadecene derivative, a pentacyclopentadecadiene derivative, a pentacyclo-3-pentadecene derivative, a pentacyclo-4-hexadecene derivative, a pentacyclo-3-hexadecene derivative, a hexacyclo-4-heptadecene derivative, a heptacyclo-5-eicosene derivative, a heptacyclo-4-eicosene derivative, a heptacyclo-5-heneicosene derivative, an octacyclo-5-dococene derivative, a nonacyclo-5-pentacosene derivative, a nonacyclo-6-hexacosene derivative, a derivative of cyclopentadiene-acenaphthylene adduct, a 1,4-methano-1,4,4a,9a-tetrahydrofluorene derivative, a 1,4-methano-1,4,4a,5,10,10a-hexahydroanthracene derivative, a cycloalkylene derivative having 3 to 20 carbon atoms, and the like.

Further, among the structural units (B) derived from the cyclic olefin represented by the above General Formula (11), (12), or (13), a tetracyclo[4.4.0.1^{2,5}.1^{7,10}]-3-dodecene derivative, a hexacyclo[6.6.1.1^{3,6}.1^{10,13}.0^{2,7}.0^{9,14}]-4-heptadecene derivative, and the derivatives of the compound represented by the following structures are exemplified as the preferred embodiments.

### 5-Phenyl-bicyclo[2.2.1]hept-2-ene

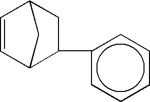

### 5-Methyl-5-phenyl-bicyclo [2.2.1] hept-2-ene

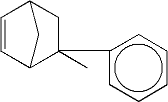

### 5-Tolyl-bicyclo [2.2.1] hept-2-ene

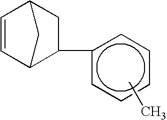

### 5-(Ethylphenyl)-bicyclo[2.2.1]hept-2-ene

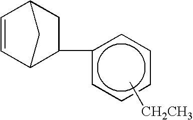

### 5-(Isopropylphenyl)-bicyclo[2.2.1] hept-2-ene

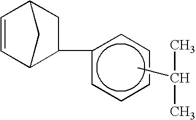

### 5-(α-Naphtyl)-bicyclo [2.2.1] hept-2-ene

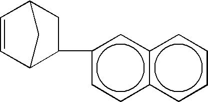

### 5-(Biphenyl)-bicyclo[2.2.1]hept-2-ene

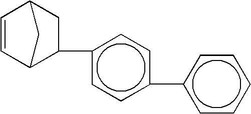

### 5,6-(Diphenyl)-bicyclo[2.2.1] hept-2-ene

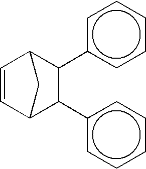

### 1,4-Methano-1,4,4a,9a-tetrahydrofluorene

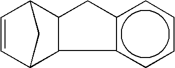

### 1,4-Methano-1,4,4a,5,10,10a-hexahydroanthracene

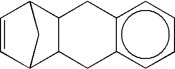

### Cyclopentadiene-acenaphthylene Adduct

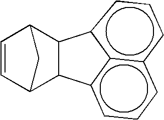

### Cyclopentadiene-benzaine Adduct (benzonorbornadiene)

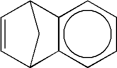

### Benzonorbornadiene Derivative

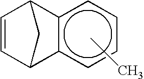

Further, particularly preferably, the cyclic olefin is selected from the group consisting of tetracyclo[4.4.0.1^{2,5}.1^{7,10}]-3-dodecene, 1,4-methano-1,4,4a,9a-tetrahydrofluorene, cyclopentadiene-benzaine adduct, and cyclopentadiene-acenaphthylene adduct, and most preferably, it is tetracyclo[4.4.0.1^{2,5}.1^{7,10}]-3-dodecene.

The cyclic olefin represented by the above General Formula (11) or (12) can be produced by performing a Diels-Alder reaction of cyclopentadiene with an olefin having a corresponding structure. The structural unit (B) derived from the cyclic olefin represented by the above General Formula (11), (12), or (13) may be included in two or more kinds. Additionally, ones polymerized using the above monomer can be modified according to its necessity, and in such case, a structure of the structural unit derived from the monomer can be modified. For example, according to a hydrogenation treatment, a benzene ring, and the like in the structural unit derived from the monomer can be modified into a cyclohexyl ring under the condition.

In the present invention, the “(i) copolymer of ethylene or α-olefin and cyclic olefin” is preferably a copolymer comprising ethylene and tetracyclo[4.4.0.1^{2,5}. 1^{7,10}]-3-dodecene.

Further, the type of the copolymerization in the present invention is not limited at all, and well-known various types of copolymerization such as a random copolymer, a block copolymer, an alternating copolymer, and the like, can be employed, but preferred is a random copolymer.

((ii) Ring-Opening Polymer or a Hydrogenated Product Thereof)

The (ii) ring-opening polymer or a hydrogenated product thereof is a cyclic olefin polymer containing a structural unit represented by General Formula (9) among the structural groups exemplified as the preferable examples in the above General Formula (3).

Further, the cyclic olefin polymer may have a polar group. Examples of the polar group include a hydroxyl group, a carboxyl group, an alkoxy group, an epoxy group, a glycidyl group, an oxycarbonyl group, a carbonyl group, an amino group, an ester group, and the like.

The cyclic olefin polymer is generally obtained by polymerizing a cyclic olefin, specifically by ring-open polymerizing an alicyclic olefin, and for example, the cyclic olefin polymer having a polar group is obtained by introducing a compound having a polar group in the cyclic olefin polymer by a modification reaction, or copolymerizing a monomer containing a polar group as the copolymer component.

Specific examples of the alicyclic olefin used to obtain the cyclic olefin polymer include the followings. A norbornene monomer such as bicyclo[2.2.1]-hept-2-ene (popular name: norbornene), 5-methyl-bicyclo[2.2.1]-hept-2-ene, 5,5-dimethyl-bicyclo[2.2.1]-hept-2-ene, 5-ethyl-bicyclo[2.2.1]-hept-2-ene, 5-butyl-bicyclo[2.2.1]-hept-2-ene, 5-hexyl-bicyclo[2.2.1]-hept-2-ene, 5-octyl-bicyclo[2.2.1]-hept-2-ene, 5-octadecyl-bicyclo[2.2.1]-hept-2-ene, 5-ethylidene-bicyclo[2.2.1]-hept-2-ene, 5-methylidene-bicyclo[2.2.1]-hept-2-ene, 5-vinyl-bicyclo[2.2.1]-hept-2-ene, 5-propenyl-bicyclo[2.2.1]-hept-2-ene, 5-methoxy-carvinyl-bicyclo[2.2.1]-hept-2-ene, 5-cyano-bicyclo[2.2.1]-hept-2-ene, 5-methyl-5-methoxycarbonyl-bicyclo[2.2.1]-hept-2-ene, 5-ethoxycarbonyl-bicyclo[2.2.1]-hept-2-ene, bicyclo[2.2.1]-hept-5-enyl-2-methylpropionate, bicyclo[2.2.1]-hept-5-enyl-2-methyloctanate, bicyclo[2.2.1]-hept-2-ene-5,6-dicarboxylic acid anhydride, 5-hydroxymethylbicyclo[2.2.1]-hept-2-ene, 5,6-di(hydroxy methyl)-bicyclo[2.2.1]-hept-2-ene, 5-hydroxy-1-propylbicyclo[2.2.1]-hept-2-ene, 5,6-dicarboxy-bicyclo[2.2.1]-hept-2-ene, bicyclo[2.2.1]-hept-2-ene-5,6-dicarboxylic acid imide, 5-cyclopentyl-bicyclo[2.2.1]-hept-2-ene, 5-cyclohexyl-bicyclo[2.2.1]-hept-2-ene, 5-cyclohexenyl-bicyclo[2.2.1]-hept-2-ene, 5-phenyl-bicyclo[2.2.1]-hept-2-ene, tricyclo[4.3.0.1^{2,5}]deca-3,7-diene (popular name: dicyclopentadiene), tricyclo[4.3.0.1^{2,5}]deca-3-ene, tricyclo[4.4.0.1^{2,5}]undeca-3,7-diene, tricyclo[4.4.0.1^{2,5}]undeca-3,8-diene, tricyclo[4.4.0.1^{2,5}]undeca-3-ene, tetracyclo[7.4.0.1^{10,13}.0^{2,7}]-trideca-2,4,6-11-tetraene (popular name: 1,4-methano-1,4,4a,9a-tetrahydrofluorene), tetracyclo[8.4.0.1^{11,14}.0^{3,8}]-tetradeca-3,5,7,12-11-tetraene (popular name: 1,4-methano-1,4,4a,5,10,10a-hexahydroanthracene), tetracyclo[4.4.0.1^{2,5}.1^{7,10}]-dodeca-3-ene (popular name: tetracyclododecene), 8-methyl-tetracyclo[4.4.0.1^{2,5}.1^{7,10}]-dodeca-3-ene, 8-ethyl-tetracyclo[4.4.0.1^{2,5}.1^{7,10}]-dodeca-3-ene, 8-methylidene-tetracyclo[4.4.0.1^{2,5}.1^{7,10}]-dodeca-3-ene, 8-ethylidene-tetracyclo[4.4.0.1^{2,5}.1^{7,10}]-dodeca-3-ene, 8-vinyl-tetracyclo[4.4.0.1^{2,5}.1^{7,10}]-dodeca-3-ene, 8-propenyl-tetracyclo[4.4.0.1^{2,5}.1^{7,10}]-dodeca-3-ene, 8-methoxycarbonyl-tetracyclo[4.4.0.1^{2,5}.1^{7,10}]-dodeca-3-ene, 8-methyl-8-methoxycarbonyl-tetracyclo[4.4.0.1^{2,5}.1^{7,10}]-dodeca-3-ene, 8-hydroxymethyl-tetracyclo[4.4.0.1^{2,5}.1^{7,10}]-dodeca-3-ene, 8-carboxy-tetracyclo[4.4.0.1^{2,5}.1^{7,10}]-dodeca-3-ene, 8-cyclopentyl-tetracyclo[4.4.0.1^{2,5}.1^{7,10}]-dodeca-3-ene, 8-cyclohexyl-tetracyclo[4.4.0.1^{2,5}.1^{7,10}]-dodeca-3-ene, 8-cyclohexenyl-tetracyclo[4.4.0.1^{2,5}.1^{7,10}]-dodeca-3-ene, 8-phenyl-tetracyclo[4.4.0.1^{2,5}. 1^{7,10}]-dodeca-3-ene, pentacyclo[6.5.1.1^{3,6}.0^{2,7}.0^{9,13}]-pentadeca-3,10-diene, and pentacyclo[7.4.0.1^{3,6}.1^{10,13}.0^{2,7}]-pentadeca-4,11-diene, and the like; a monocyclic cycloalkene such as cyclobutene, cyclopentene, cyclohexene, 3,4-dimethylcyclopentene, 3-methylcyclohexene, 2-(2-methylbuthyl)-1-cyclohexene, cyclooctene, 3a,5,6,7a-tetrahydro-4,7-methano-1H-indene, cycloheptene, and the like;

a vinyl alicyclic hydrocarbon monomer such as vinyl cyclohexene, vinyl cyclohexane, and the like; and

an alicyclic conjugated diene monomer such as cyclopentadiene, cyclohexadiene, and the like. The alicyclic olefin may be used each singly or in combination of two or more kinds thereof.

Additionally, a copolymerizable monomer can be copolymerized, if necessary. Specific examples of the monomer include ethylene or α-olefin having 2 to 20 carbon atoms such as ethylene, propylene, 1-butene, 1-pentene, 1-hexene, 3-methyl-1-butene, 3-methyl-1-pentene, 3-ethyl-1-pentene, 4-methyl-1-pentene, 4-methyl-1-hexene, 4,4-dimethyl-1-hexene, 4,4-dimethyl-1-pentene, 4-ethyl-1-hexene, 3-ethyl-1-hexene, 1-octene, 1-decene, 1-dodecene, 1-tetradecene, 1-hexadecene, 1-octadecene, and 1-eicosene; a cycloolefin such as cyclobutene, cyclopentene, cyclohexene, 3,4-dimethylcyclopentene, 3-methylcyclohexene, 2-(2-methylbutyl)-1-cyclohexene, cyclooctene, 3a,5,6,7a-tetrahydro-4,7-methano-1H-indene, and the like; and a nonconjugated diene such as 1,4-hexadiene, 4-methyl-1,4-hexadiene, 5-methyl-1,4-hexadiene, 1,7-octadiene, and the like. These monomers may be used each singly or in combination of two or more kinds thereof.

A polymerization method of the alicyclic olefin is not particularly limited, and can be carried out in accordance with a well-known method. These ring-opening polymerization products are preferably used as a hydrogenated product, from the viewpoint of the thermal resistance, the stability, and the optical properties. As for the hydrogenation method, well-known methods can be used.

((iii) Vinyl Alicyclic Hydrocarbon Polymer)

The (iii) vinyl alicyclic hydrocarbon polymer is a hydrogenated product of a (co)polymer obtained from a vinyl aromatic hydrocarbon compound as a monomer, or a (co) polymer obtained from vinyl alicyclic hydrocarbon compound as a monomer. Examples of the vinyl compound include a vinyl aromatic compound, a vinyl alicyclic hydrocarbon compound, and the like.

Examples of the vinyl aromatic compound include styrenes such as styrene, α-methylstyrene, α-ethylstyrene, α-propylstyrene, α-isopropylstyrene, α-t-butylstyrene, 2-methylstyrene, 3-methylstyrene, 4-methylstyrene, 2,4-diisopropylstyrene, 2,4-dimethylstyrene, 4-t-butylstyrene, 5-t-butyl-2-methylstyrene, monochlorostyrene, dichlorostyrene, monofluorostyrene, 4-phenylstyrene, and the like.

Examples of the vinyl alicyclic hydrocarbon compound include vinylcyclohexanes such as vinylcyclohexane, 3-methylisopropenylcyclohexane, and the like; and vinylcyclohexenes such as 4-vinylcyclohexene, 4-isopropenylcyclohexene, 1-methyl-4-vinylcyclohexene, 1-methyl-4-isopropenylcyclohexene, 2-methyl-4-vinylcyclohexene, 2-methyl-4-isopropenylcyclohexene, and the like.

In the present invention, the above-described monomers and the other copolymerizable monomers may be copolymerized. Examples of the copolymerizable monomer include α-olefin monomers such as ethylene, propylene, isobutene, 2-methyl-1-butene, 2-methyl-1-pentene, 4-methyl-1-pentene, and the like; cyclopentadiene monomers such as cyclopentadiene, 1-methylcyclopentadiene, 2-methylcyclopentadiene, 2-ethylcyclopentadiene, 5-methylcyclopentadiene, 5,5-dimethylcyclopentadiene, dicyclopentadiene, and the like; monocyclic olefin monomers such as cyclobutene, cyclopentene, cyclohexene, and the like; conjugated diene monomers such as butadiene, isoprene, 1,3-pentadiene, furane, thiophene, 1,3-cyclohexadiene, and the like; nitrile monomers such as acrylonitrile, methacrylonitrile, α-chloroacrylonitrile, and the like; (meth)acrylate ester monomers such as methyl methacrylate, ethyl methacrylate, propyl methacrylate, butyl methacrylate, methyl acrylate, ethyl acrylate, propyl acrylate, butyl acrylate, and the like; unsaturated fatty acid monomers such as acrylic acid, methacrylic acid, maleic acid anhydride, and the like; phenylmaleimide, methylvinyl ether, and heterocyclic ring-containing vinyl compound monomers such as N-vinylcarbazole, N-vinyl-2-pyrolidone, and the like.

From the viewpoints of thermal resistance, low birefringence, and mechanical strength, the mixture of above-mentioned monomers used for polymerization contains generally a vinyl aromatic compound and/or a vinyl alicyclic hydrocarbon compound in the amount of 50% by mass or more, preferably from 70 to 100% by mass, and even more preferably from 80 to 100% by mass. The monomer mixture may contain both the vinyl aromatic compound and the vinyl alicyclic hydrocarbon compound.

A polymerization method of the vinyl aromatic hydrocarbon compound or the vinyl alicyclic hydrocarbon compound is not particularly limited, and can be carried out in accordance with a well-known method. The (co)polymer obtained from the vinyl aromatic hydrocarbon compound is preferably used as a hydrogenated product, considering the thermal resistance, the stability, and the optical properties. As for the hydrogenation method, well-known methods can be used.

The hydrogenated product of the (co)polymer obtained from the vinyl aromatic hydrocarbon compound can have a hydrogenation rate of phenyl groups of preferably 95% or more, and more preferably 99% or more. By hydrogenation treatment, the phenyl groups in the resin structure changes to cyclohexyl groups. The molded product containing the resin has an improved light transmittance at a short wavelength and a reduced birefringence/optical anisotropy. Also, by hydrogenation treatment of unreacted monomers and impurities at the same time, resistance against heat/light is improved. By treating a hydrogenation rate within the above value range, these effects are particularly remarkable.

((iv) Other Polymers)

Specific examples of (iv) the other polymer include a polymer of monocyclic cycloalkene, a polymer of alicyclic conjugated diene monomer, and an aromatic olefin polymer. The structure which is not contained in the above (i) to (iii) may also be optionally selected within the above range of General Formula (3). Examples include the ones obtained by copolymerization of (i) to (iii), or copolymerization of well-known copolymerizable monomers.

Further, the type of the copolymerization in the present invention is not limited at all, and well-known various types of copolymerization such as a random copolymer, a block copolymer, an alternating copolymer, and the like can be employed, but preferred is a random copolymer.

Among the four kinds of polymers classified into the above mentioned (i) to (iv), (i) the copolymer of ethylene or α-olefin, and cyclic olefin is preferable, and among them, an ethylene tetracyclo[4.4.0.1^{2,5}.1^{7,10}]-3-dodecene copolymer is most preferable, from the viewpoint of optical properties.

(Other Structure which can be Used as a Part of a Main Chain)

The polymer having an alicyclic structure which is used in the present invention may have a repeating structural unit derived from the other copolymerizable monomer within the above range of not impairing the preferred properties of the product obtained by the molding method of the present invention, depending on its necessity. The copolymerization ratio is not limited, but is preferably 20 mol % or less and more preferably 0 to 10 mol %, and if the copolymerization amount is 20 mol % or less, a high precision optical component can be obtained without impairing the optical properties. In addition, the kind of copolymerization is not limited.

(Molecular Weight of Polymer Having an Alicyclic Structure at Least in a Part of a Repeating Structural Unit)

The molecular weight of the polymer having an alicyclic structure used in the present invention is not limited, but when the limiting viscosity [η] is represented as an alternative characteristic of a molecular weight, the limiting viscosity [η] measured in the decalin at 135° C. is preferably from 0.03 to 10 dl/g, more preferably from 0.05 to 5 dl/g, and most preferably from 0.10 to 2 dl/g. When the limiting viscosity [η] is within the above value range, an excellent moldability can be obtained, and the mechanical strength of the molded product is not impaired.

### (Glass Transition Temperature of the Polymer Having an Alicyclic Structure at Least in a Part of a Repeating Structural Unit)

The glass transition temperature (Tg) of the polymer having an alicyclic structure at least in a part of a repeating structural unit used in the present invention is preferably from 50 to 240° C., more preferably from 50 to 160° C., and most preferably from 100 to 150° C. When the glass transition temperature (Tg) is within the above value range, during the use of the molded product as an optical component, a sufficient thermal resistance can be obtained, and also, an excellent moldability is obtained.

The measuring apparatus of the glass transition temperature, and the like are not limited, but for example, the differential scanning calorimeter (DSC) can be used to measure the glass transition temperature of a thermoplastic amorphous resin. For example, a measurement method with the use of DSC-20 manufactured by SEIKO Corporation and at the rate of the temperature increase of 10° C./minute, or the like is exemplified.

Such polymers with alicyclic structures can be produced by suitably selecting the condition in accordance with the method as described below, respectively.

(i) JP-A-60-168708, JP-A-61-120816, JP-A-61-115912, JP-A-61-115916, JP-A-61-271308, JP-A-61-272216, JP-A-62-252406, and JP-A-62-252407 for the copolymer of ethylene or α-olefin and cyclic olefin;

(ii) JP-A-60-26024, JP-A-9-268250, JP-A-63-145324, and JP-A-2001-72839 for the ring-opening polymer or a hydrogenated product thereof; and

(iii) WO 01/092412, and JP-A-2003-276047 and 2004-83813 for the vinyl alicyclic hydrocarbon polymer.

Moreover, in the process for producing the above polymer having an alicyclic structure, at least once, by contacting a hydrogenation catalyst and hydrogen into a system containing the polymer or the polymer with a monomer which is a starting material, and then hydrogenating at least one part of an unsaturated bond included in the polymer and/or monomer, it is possible to improve the optical properties of the polymer such as the thermal resistance and the transparency. Here, the hydrogenation so-called a hydrogenation can be carried out in accordance with a well-known conventional method.

### [Hindered Amine-Based Compound]

The hindered amine compound used in the present invention can have a ratio of carbon atoms in the molecule structure from 67% by weight to 80% by weight, preferably from 68% by weight to 79% by weight, and more preferably 70% by weight to 77% by weight.

If the ratio of carbon atoms contained in the hindered amine compound is no less than the lower limit, a resin composition in which the hindered amine compound is sufficiently dispersed can be obtained. As a result, a molded product such as an optical component obtained by molding the resin composition can sufficiently exhibit light resistance, and accordingly, the change in the shapes and the refractive index during the use is suppressed, and further, the generation of the microcracks can be suppressed. On the other hand, if the ratio of carbon atoms contained in the hindered amine compound is no more than the upper limit, the density of the functional groups of the hindered amine compound in the resin composition becomes sufficient, and as a result, more excellent light resistance can be exhibited. That is, by using the ratio of carbon atoms in the molecule structure within the above value range, a molded product such as an optical component having excellent light resistance can be obtained. As a result, in the use as an optical component, the deterioration of the optical characteristics can be suppressed, and in particular, in the use of a blue-violet laser light source, the deterioration of the optical characteristics can be effectively suppressed.

Further, the ratio of carbon atoms contained in the molecule structure as described above is a theoretical value as calculated from the chemical formula, but this theoretical value is substantially consistent to the ratio of carbon atoms as measured by a CHN elemental analyzer (for example, CHNS-932 manufactured by LECO Corporation).

Also, the molecular weight of the hindered amine compound used in the present invention can be from 500 to 3500, preferably from 600 to 3000, and more preferably from 700 to 2000.

If the molecular weight of the hindered amine compound is no less than the lower limit, the movement of the hindered amine compound is suppressed in the resin after molding. As a result, the light resistance is sufficiently exhibited, and accordingly, the change in the shapes and the refractive index during the use is suppressed, and further, the generation of the microcracks can be suppressed. On the other hand, if the molecular weight of the hindered amine compound is no more than the upper limit, the fluidity upon melting becomes sufficient, and the compound can be uniformly dispersed in the resin. As a result, the light resistance is sufficiently exhibited, and accordingly, the change in the shapes and the refractive index during the use is suppressed, and further, the generation of the microcracks can be suppressed. That is, by using the hindered amine compound within the above value range of the molecular weight, both of at the molding and after the molding, the dispersibility of the hindered amine compound is excellent, and accordingly, the change in the shapes and the refractive index of the molded product during the use is effectively suppressed, and the generation of the microcracks can be effectively prevented.

In addition, the molecular weight of the above-described hindered amine compound is a theoretical value as calculated from the chemical formula, but this theoretical value is substantially consistent to a weight average molecular weight in terms of polystyrene as measured by gel permeation chromatography (GPC), or a molecular weight as measured by mass analysis.

According to the hindered amine compound used in the present invention, both of the ratio of carbon atoms and the molecular weight are within the above value range, and thus by using the compound in a predetermined amount, the dispersibility in the resin composition can be improved, and a molded product having excellent light resistance, transparency, or the like, and suppressed deterioration of the optical characteristics during the use of a blue-violet laser light source can be obtained.

In the resin composition of the present invention, by using the hindered amine compound and the polymer represented by General Formula (3), an optical component having low reduction in the light transmittance and low deterioration in optical performances during the use of a blue-violet laser light source while maintaining moldability, low birefringence, heat resistance, mass productivity, mechanical strength, and light transmittance can be obtained. The optical component and the optical pickup device, comprising the resin composition, have sufficient optical performance, hardly deteriorate with a laser light close to ultraviolet ray, and hardly change the performances during the use, whereby it has industrially high value.

Examples of the hindered amine compound satisfying the above-described characteristics include the compounds represented by the following Chemical Formulae [1] to [43].

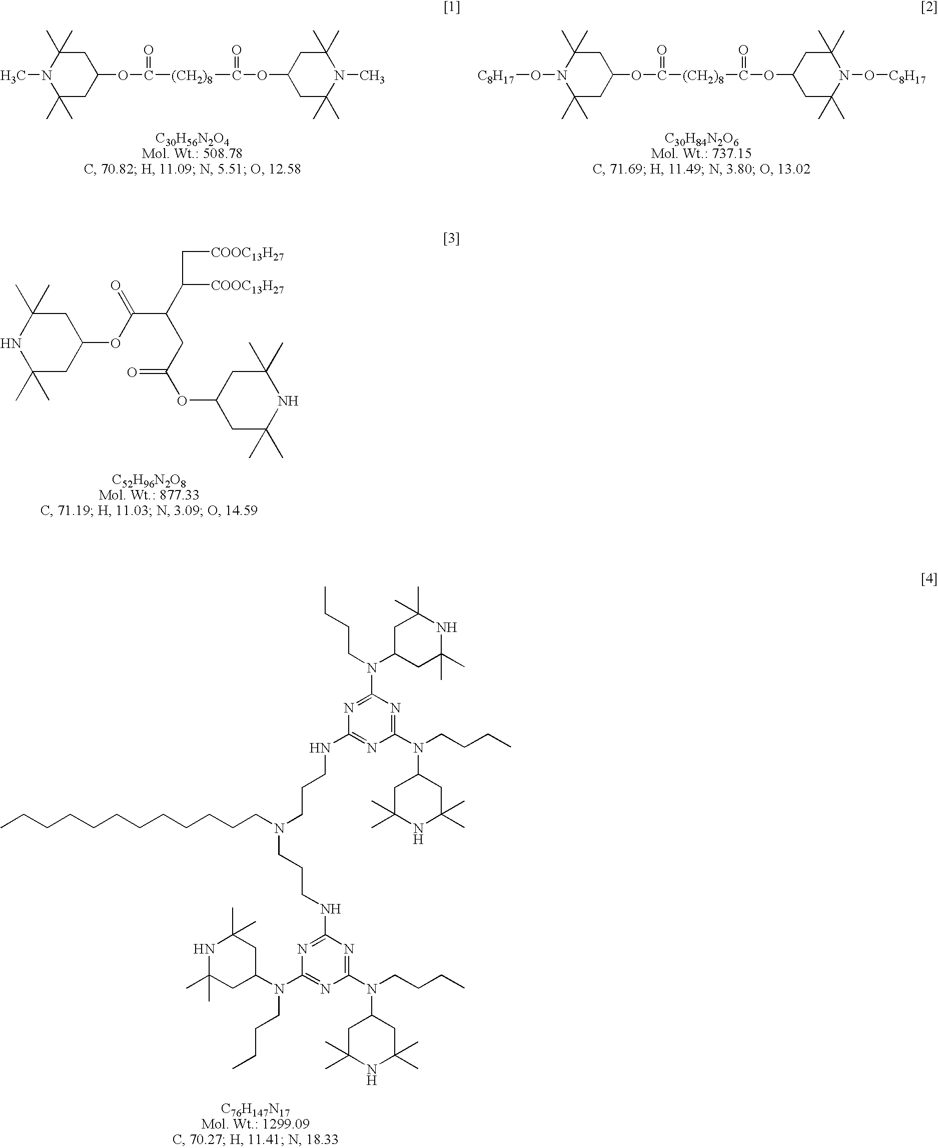

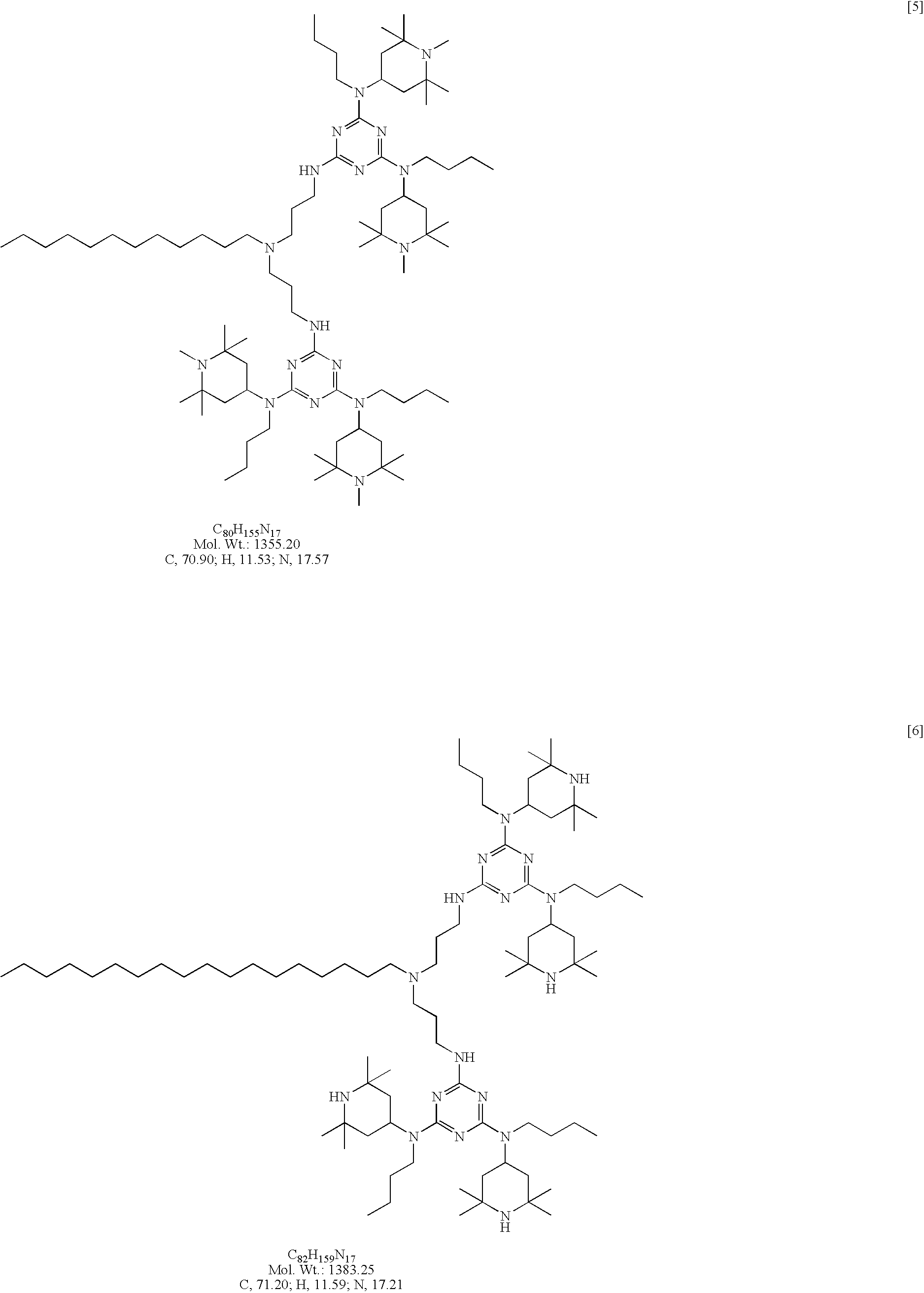

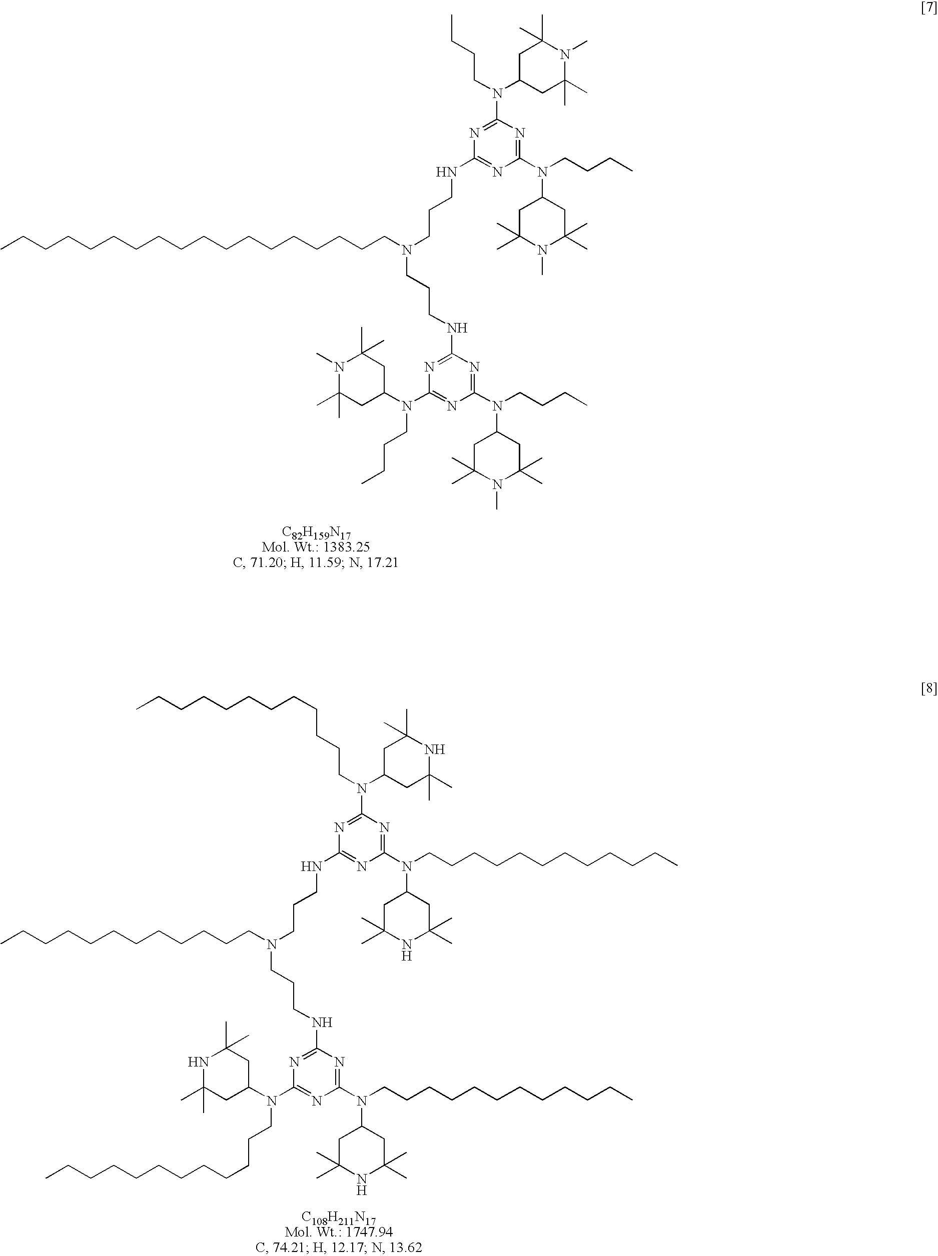

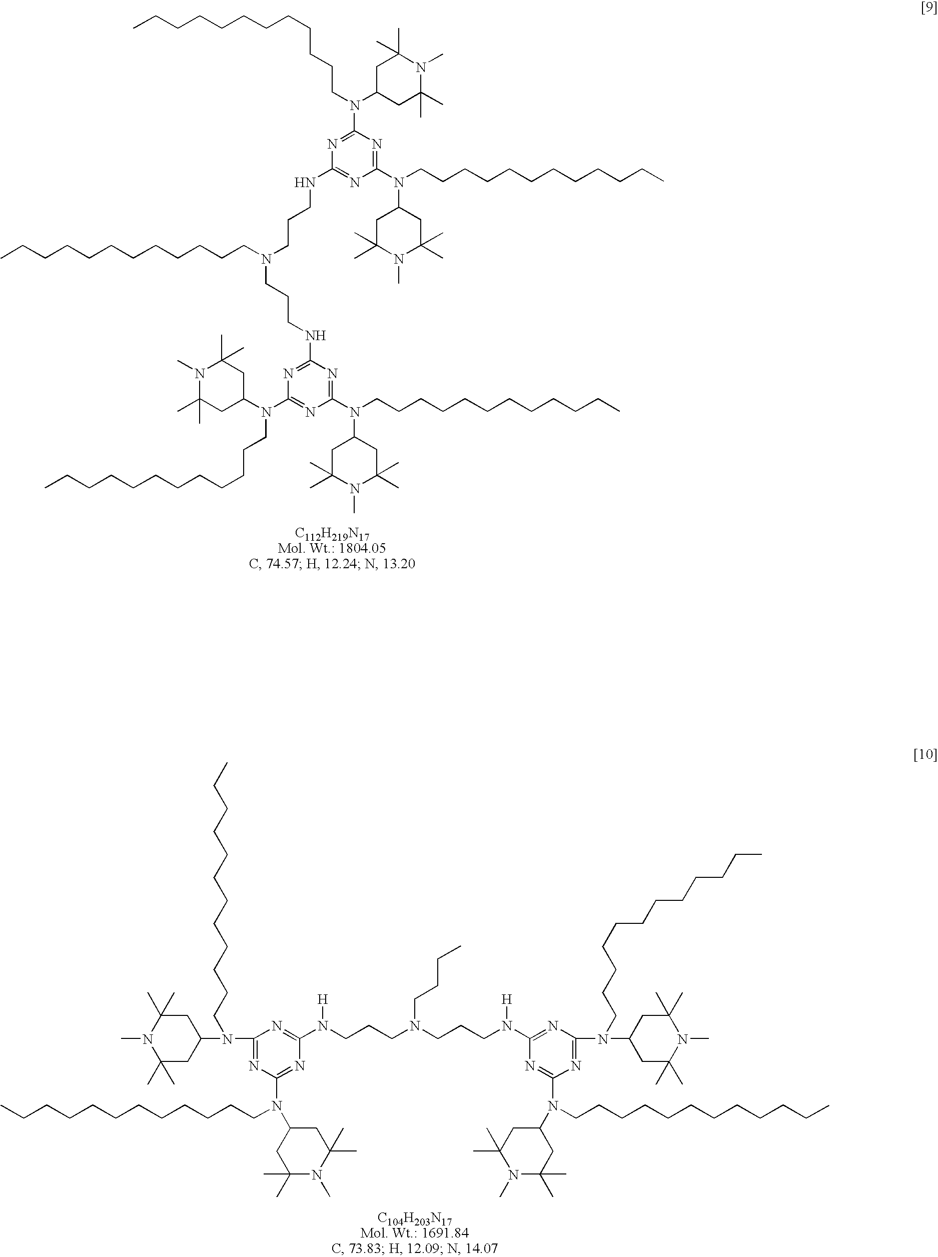

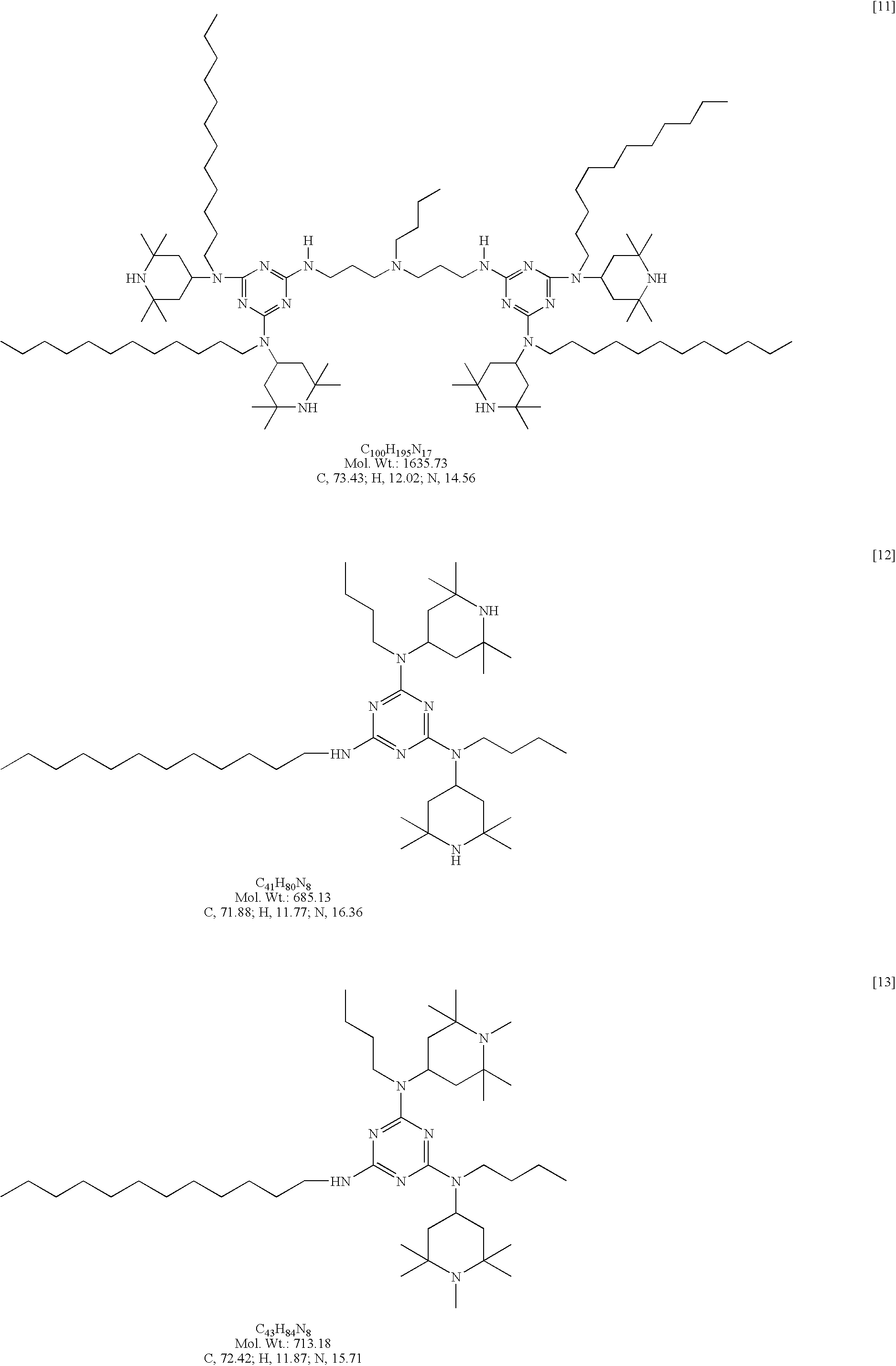

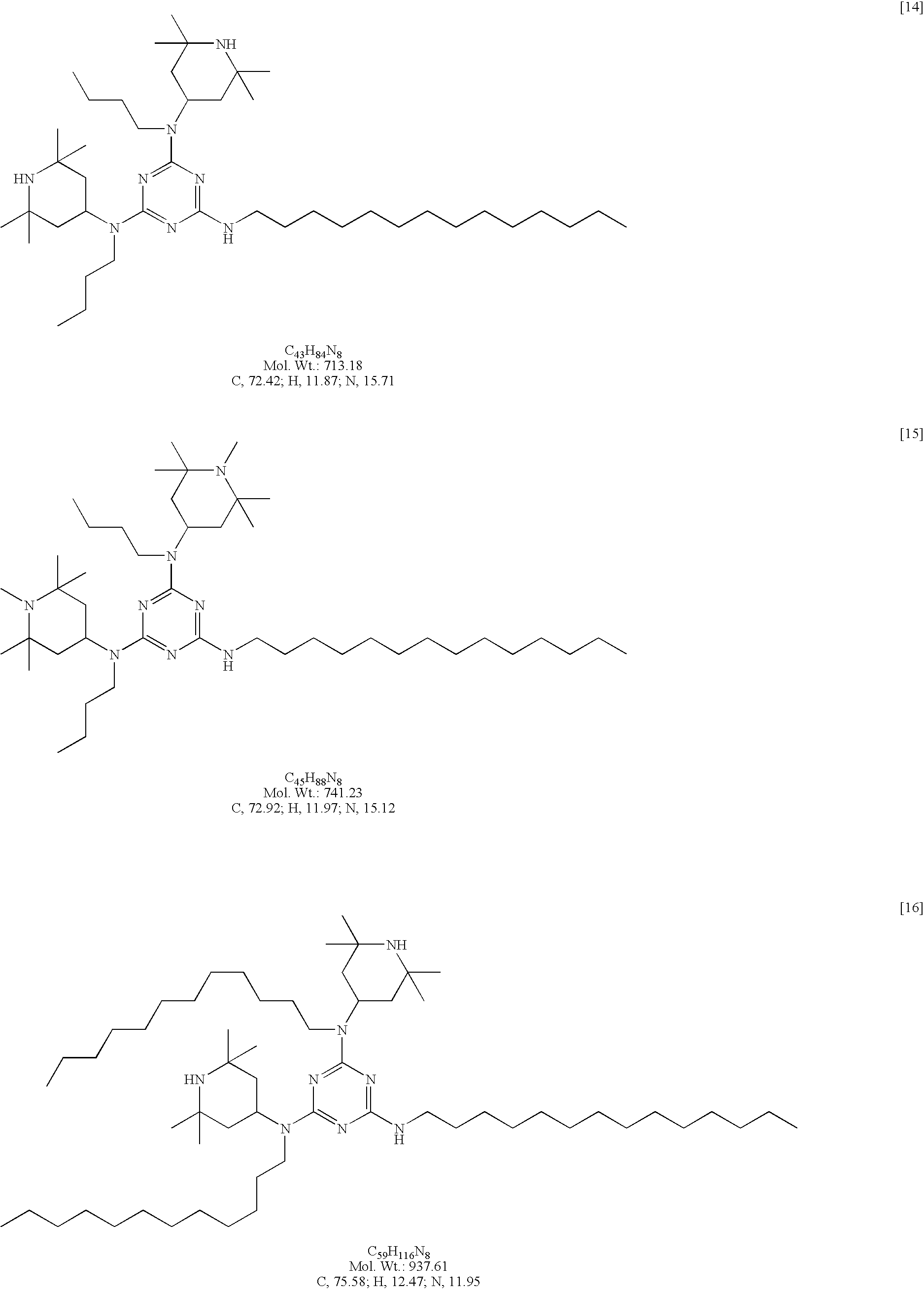

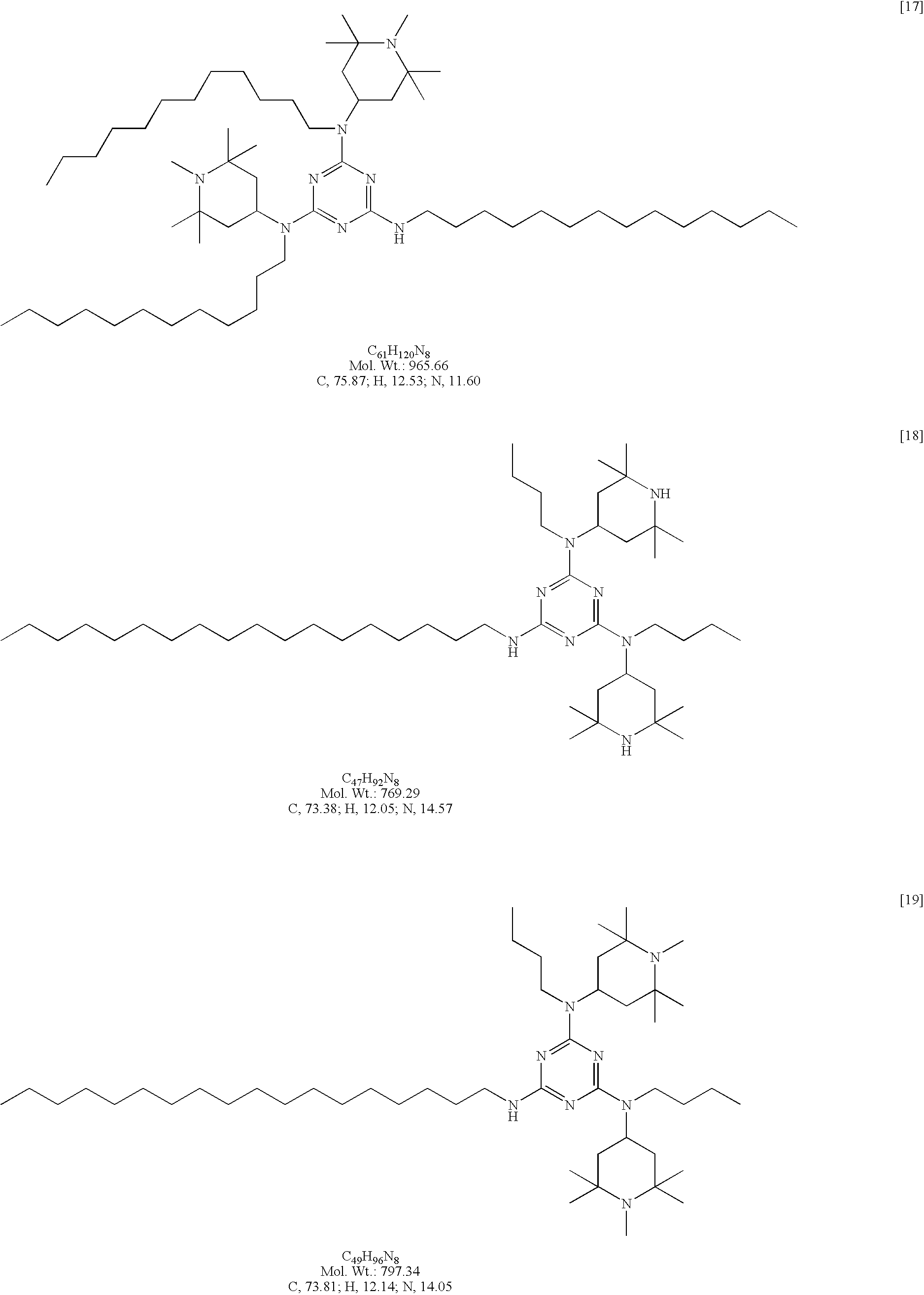

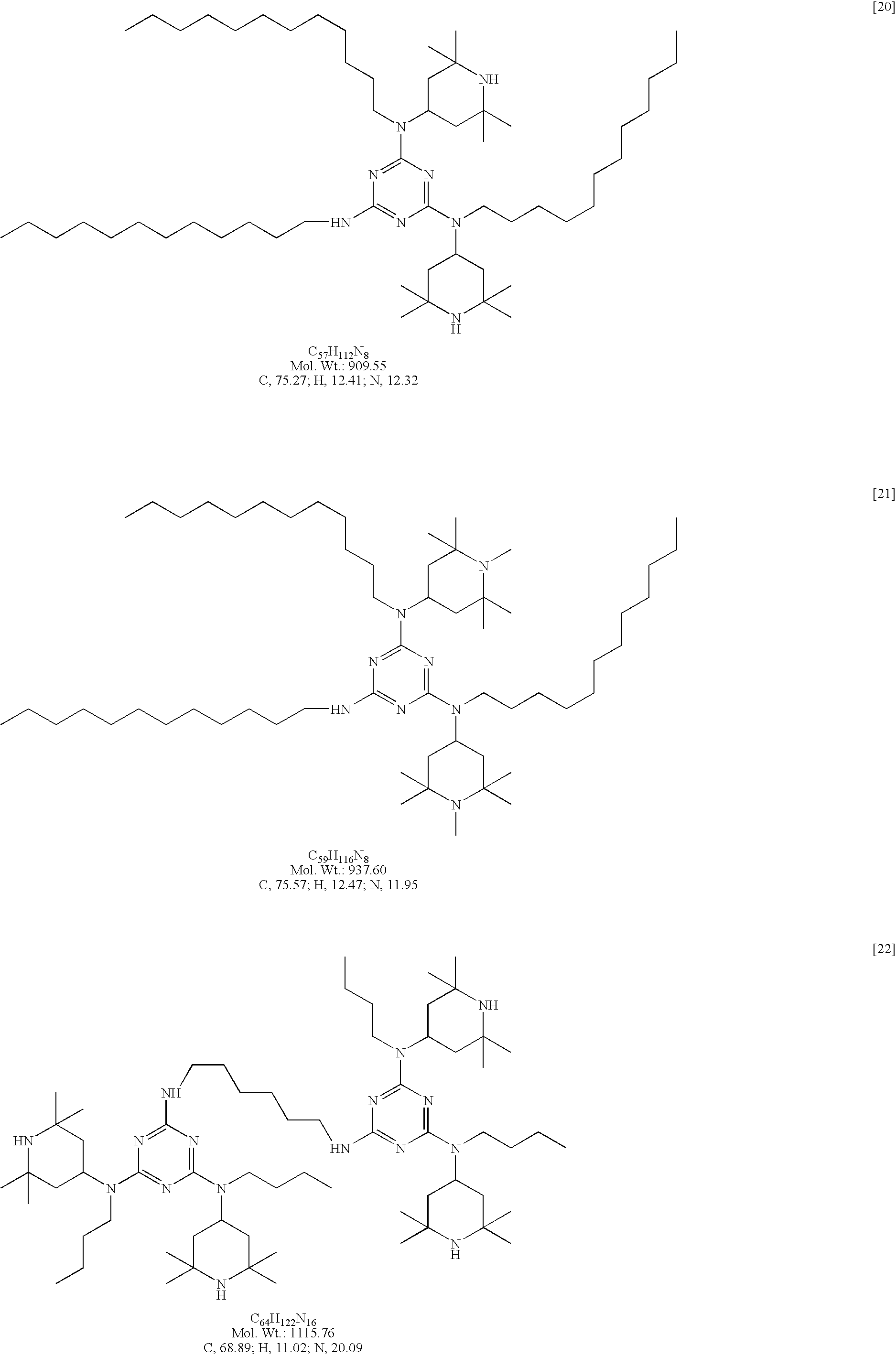

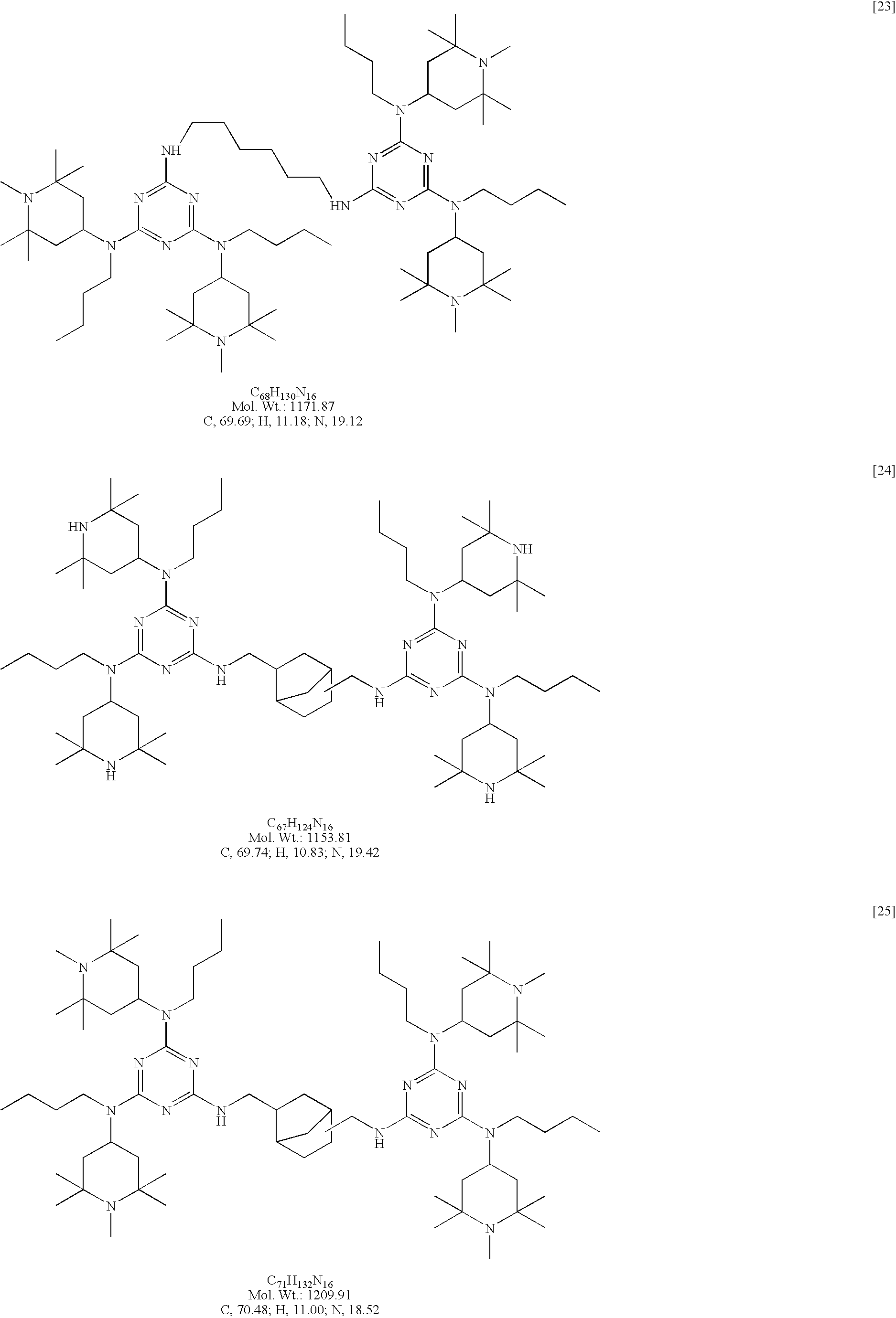

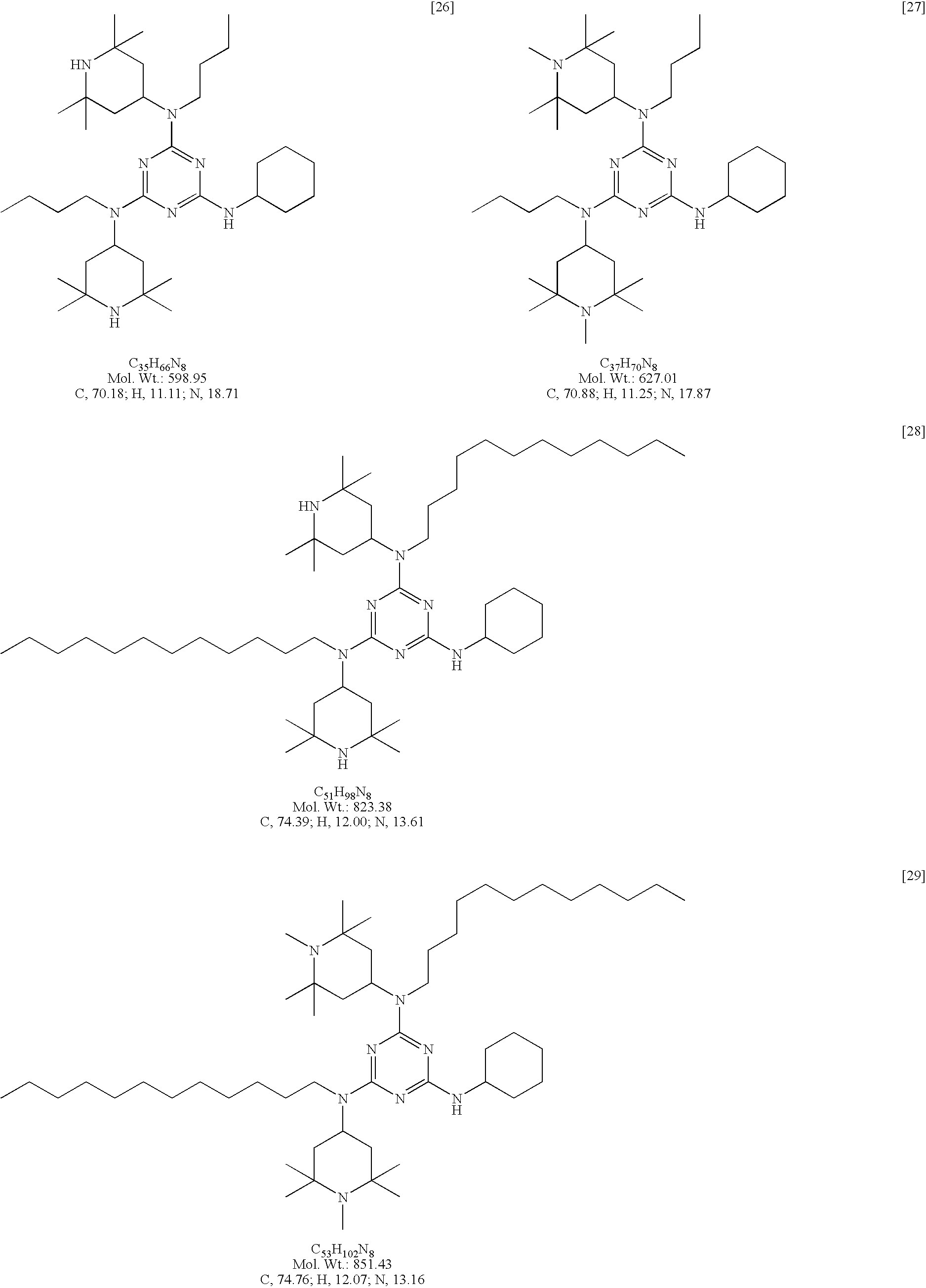

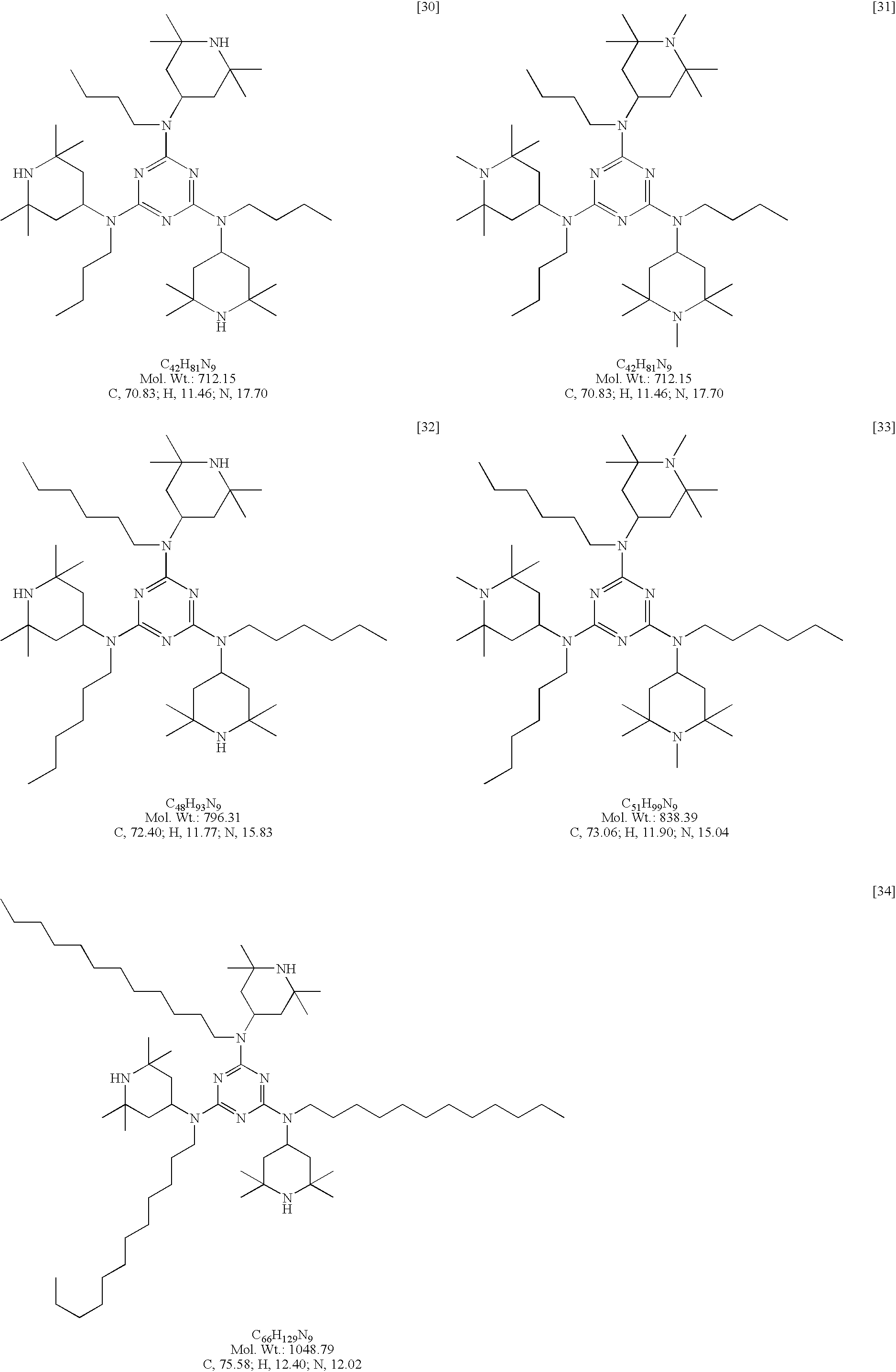

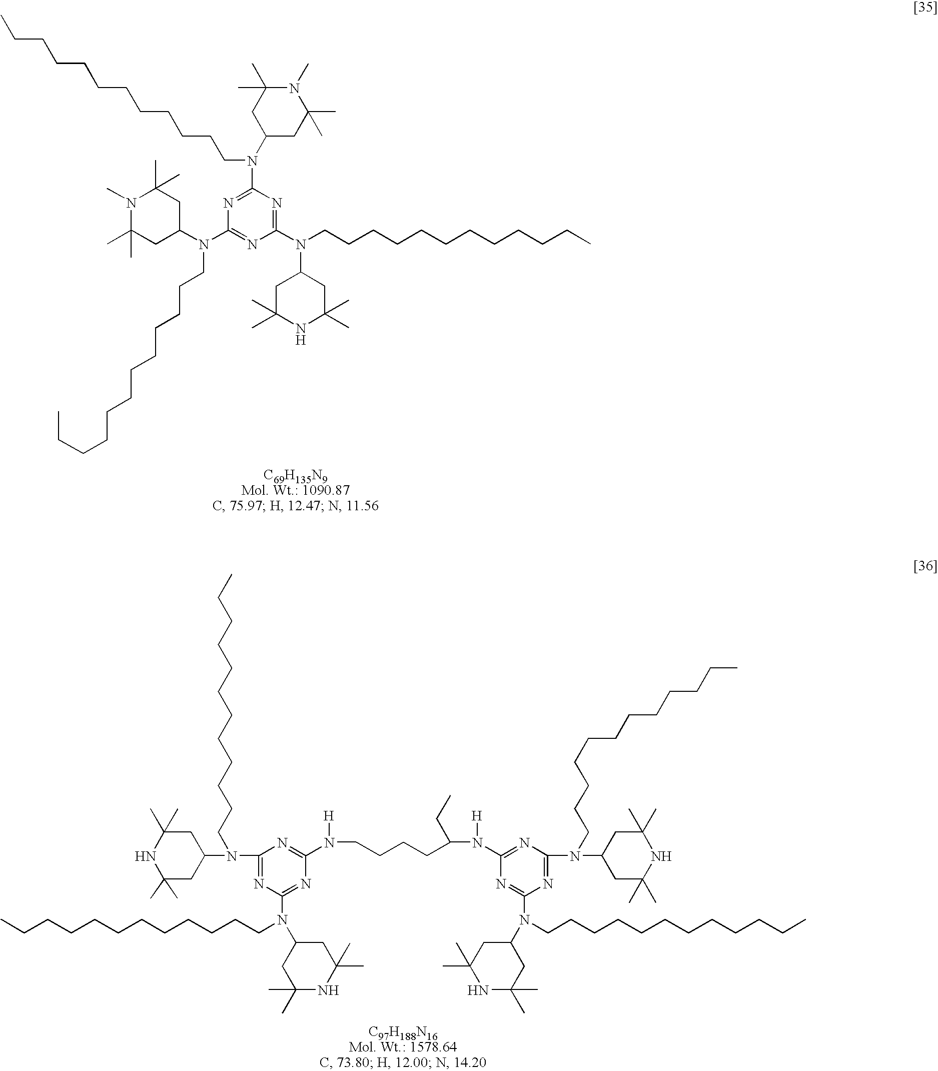

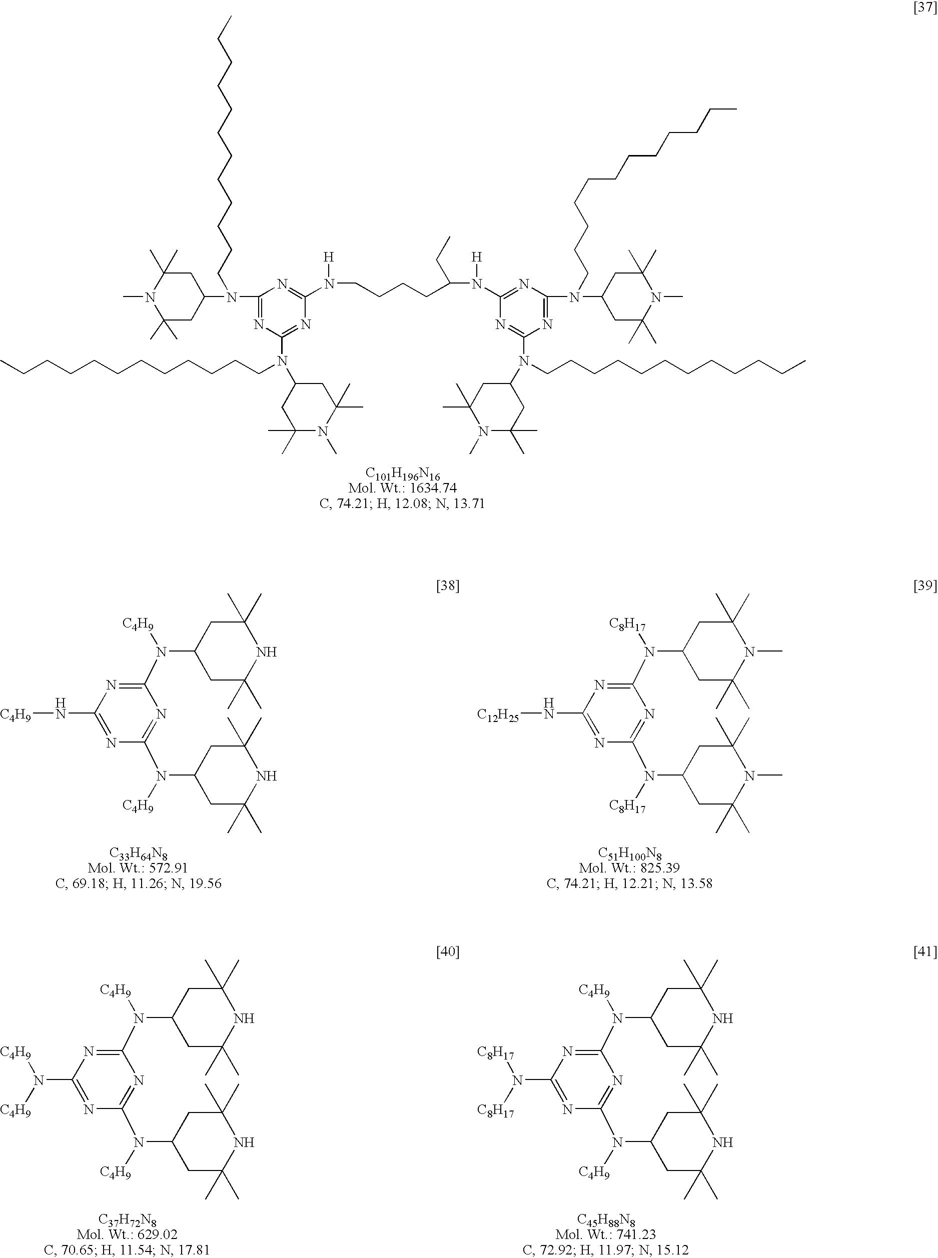

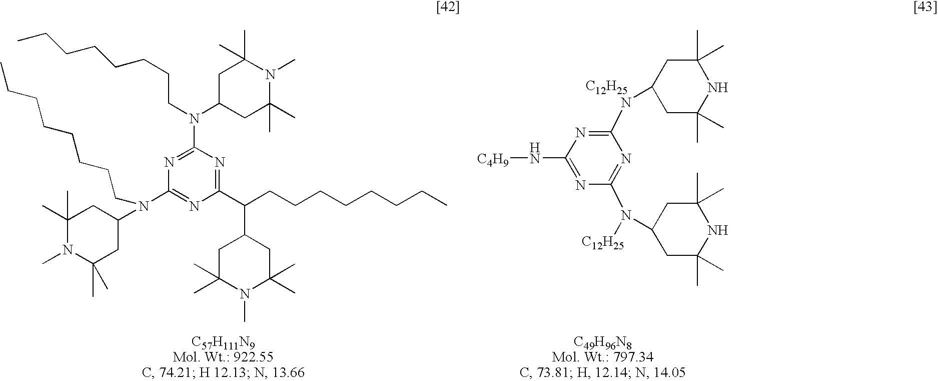

Moreover, the solubility of the hindered amine compound in 100 g of hexane at 23° C. is 25 g or more, preferably 50 g or more, and more preferably 100 g or more.

With the hexane solubility within the above value range, the dispersibility in the resin becomes sufficient. As a result, the light resistance is sufficiently exhibited, and accordingly, the change in the shapes and the refractive index during the use is suppressed, and further, the generation of the microcracks is suppressed.

Examples of the hindered amine compound satisfying the above-described hexane solubility include the compounds represented by the Chemical Formulae [1] to [43] described above.

If the hexane solubility of the hindered amine compound is within the above range, the compound is more uniformly dispersed in the resin components, and due to its light resistance, an excellent molded product can be obtained.

Moreover, when the hindered amine compound used in the present invention is heated at 5° C./minute under nitrogen, the 5% by weight reducing temperature at heating can be 300° C. or higher, and preferably 320° C. or higher. More preferably, when it is heated at 5° C./minute under nitrogen, the 1% by weight reducing temperature at heating can be 200° C. or higher, and more preferably the 5% by weight reducing temperature at heating can be 320° C. or higher, and also, the 1% by weight reducing temperature at heating can be 200° C. or higher.

If the weight reducing temperature at heating of the hindered amine compound is no less than the lower limit, the degradation of the hindered amine compound during the resin melting is suppressed. As a result, the transparency and the light resistance are sufficiently exhibited, and accordingly, the change in the shapes and the refractive index during the use is suppressed, and thus, the generation of the microcracks is also suppressed.

The weight reducing temperature at heating can be measured, for example, by means of Thermogravimetry/Differential Thermal Analysis Apparatus (TG/DTA apparatus, for example, DTG-60A/60AH manufactured by Shidmazu Corporation).

The hindered amine compound having the above-described characteristics can be represented by the following General Formula (1).

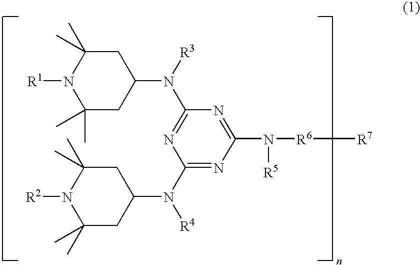

In Formula (1), n represents 1 or 2.

R¹ and R² may be the same as or different from each other, and each represent a hydrogen atom or a methyl group, and preferably a methyl group. When R¹ and R² are methyl groups, the coloration of the molded product can be prevented at a high temperature and in the coexistence of an acidic material.

R³, R⁴ and R⁵ may be the same as or different from each other, and each can be exemplified by the following (1) to (5).

(1) Hydrogen atom.

(2) Alkyl group having 1 to 24 carbon atoms.

(3) Saturated hydrocarbon group having an alicyclic skeleton having 5 to 12 carbon atoms, in which the alicyclic skeleton may have 1 to 3 alkyl substituents having 1 to 9 carbon atoms.

Examples of the saturated hydrocarbon group having an alicyclic skeleton include a cycloalkyl group having 5 to 12 carbon atoms, which may be unsubstituted or contain 1 to 3 alkyl groups having 1 to 4 carbon atoms.

(4) Group represented by —R^{A}-Ph(−R^{B})p (wherein R^{A} represents an alkylene group having 1 to 3 carbon atoms, and Ph represents a phenyl group that is unsubstituted or substituted with an alkyl group having 1 to 4 carbon atoms represented by R^{B}. p is an integer of 0 to 3.).

(5) Substituted alkyl group having 2 to 4 carbon atoms, which has at least one substituent on a carbon atom other than the carbon atom to which a nitrogen atom is directly bonded, in which the substituent is selected from an OH group, an alkoxy group having 1 to 8 carbon atoms, and a dialkylamino group (a plurality of the alkyl groups, may be the same as or different from each other, and are each an alkyl group having 1 to 4 carbon atoms).

R³, R⁴, and R⁵ may be the same as or different from each other, but for these, preferably (1) a hydrogen atom, (2) an alkyl group having 1 to 24 carbon atoms, or (3) a cycloalkyl group having 5 to 12 carbon atoms that is unsubstituted, or has 1 to 3 alkyl groups having 1 to 4 carbon atoms can be used. By using these groups as R³, R⁴, and R⁵, the transmittance at a short wavelength becomes better, and thus it can be suitably used, particularly, as an optical component.

R⁶ represents an alkylene group having 1 to 4 carbon atoms, or a single bond.

R⁷ may be the same as or different from each other, and can be exemplified by the following (1) to (7).

(1) Hydrogen atom.

(2) Aliphatic saturated hydrocarbon group having 1 to 17 carbon atoms.

The aliphatic saturated hydrocarbon group having 1 to 17 carbon atoms represents, in a case of n=1, a hydrogen atom or an alkyl group having 1 to 17 carbon atoms, or in a case of n=2, an alkylene group having 1 to 17 carbon atoms.

(3) The saturated hydrocarbon group having an alicyclic skeleton having 5 to 12 carbon atoms, in which the alicyclic skeleton may have 1 to 3 alkyl substituents having 1 to 4 carbon atoms.

The saturated hydrocarbon group having an alicyclic skeleton is, in a case of n=1, a monovalent group, or in a case of n=2, a divalent group. Examples of the monovalent group include a cyclohexyl group, and the like, and examples of the divalent group include 1,2-cyclohexylene, 1,3-cyclohexylene, 1,4-cyclohexylene, and the like.

(4) Group represented by —R^{7A}-Ph(—R^{7B})p (wherein R^{7A} represents a divalent or trivalent saturated hydrocarbon group having 1 to 3 carbon atoms, and Ph represents a phenyl group that is unsubstituted or substituted with an alkyl group having 1 to 4 carbon atoms represented by R^{7B}. p is an integer of 0 to 3.).

(5) N,N-dialkylamino group represented by —N(R^{7F})(R⁷) (wherein R^{7F} and R^{7G} each independently represent an alkyl group having 1 to 18 carbon atoms), or a group represented by —N(R^{7F})— (wherein R^{7F} represents an alkyl group having 1 to 18 carbon atoms, and — represents a bond).

(6) Substituted aliphatic saturated hydrocarbon group having 2 to 4 carbon atoms, which has at least one substituent on a carbon atom other than the carbon atom to which R⁶ is directly bonded, in which the substituent is selected from an OH group, an alkoxy group having 1 to 8 carbon atoms, and a dialkylamino group (a plurality of the alkyl groups may be the same as or different from each other, and are each an alkyl group having 1 to 4 carbon atoms).

Furthermore, the above-described substituted aliphatic saturated hydrocarbon group has a substituent on a carbon atom other than the carbon atom to which a nitrogen atom is directly bonded, wherein R⁶ is a single bond.

(7) Group represented by the following formula:

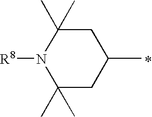

(wherein R⁸ represents a hydrogen atom or a methyl group, and * represents a bond).

As R⁷ in General Formula (1), in a case of n=1, (1) a hydrogen atom, (2) an alkyl group having 1 to 17 carbon atoms, (3) a cycloalkyl group having 5 to 12 carbon atoms that is unsubstituted, or has 1 to 3 alkyl groups having 1 to 4 carbon atoms, (5) an N,N-dialkylamino group represented by —N(R^{7F})(R^{7G}) (wherein R^{7F} and R^{7G} represent each independently an alkyl group having 1 to 18 carbon atoms), and (7) a group represented by the above Formula can be preferably used. On the other hand, in a case of n=2, as R⁷, (2) an alkylene group having 1 to 17 carbon atoms, (3) a cycloalkylene group having 5 to 12 carbon atoms that is unsubstituted, or has 1 to 3 alkyl groups having 1 to 4 carbon atoms, and (5) a group represented by —N(R^{7F})— (wherein R^{7F} represents an alkyl group having 1 to 18 carbon atoms, and — represents a bond) can be preferably used.

By using these groups as R⁷, the transmittance at a short wavelength becomes better, and thus it can be suitably used, particularly, as an optical component.

As the hindered amine compound represented by the above General Formula (1), the compounds represented by the above Chemical Formulae [4] to [43] can be exemplified.

Further, as the hindered amine compound represented by the above General Formula (1), the hindered amine compound represented by the following General Formula (2) can be used. By using this hindered amine compound, the coloration of the molded product at a high temperature can be suppressed.

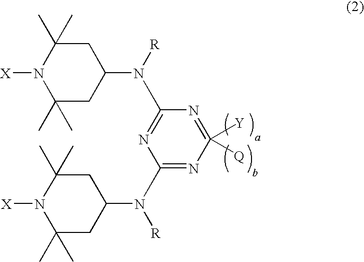

[In Formula (2), a and b are each 0 or 1, and satisfy a+b=1.

R represents an alkyl group having 1 to 24 carbon atoms.

Y is represented by the following General Formula:

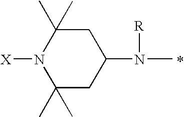

(wherein X represents a hydrogen atom or an alkyl group having 1 to 24 carbon atoms, R represents an alkyl group having 1 to 24 carbon atoms, and * represents a bond).

Q is represented by the following General Formula:

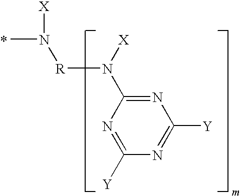

(wherein m is 0 or 1, and X and Y are the same as above. R represents, in a case of m=0, an alkyl group having 1 to 24 carbon atoms, or in a case of m=1, an alkylene group having 1 to 24 carbon atoms. * represents a bond).

A plurality of X, Y, and R may be the same as or different from each other].

Examples of the hindered amine compound represented by the above General Formula (2) include the compounds represented by the above Chemical Formulae [12] to [43].

In the above General Formula (2), the X at a 4-position of the piperidinyl group is preferably a hydrogen atom or a methyl group, and more preferably a methyl group. By using this hindered amine compound, the components of lower molecular weight produced upon degradation of the hindered amine stabilizer can be suppressed, and thus the deterioration of the optical characteristics after irradiation can be suppressed.

Further, in the above General Formula (2), in a case of a=0 and b=1, X of the General Formula representing the Q of the above General Formula (2) is preferably a hydrogen atom or a methyl group. By using this hindered amine stabilizer, the purification or the handling of the hindered amine light stabilizer becomes easier, and accordingly, the quality of the resulting resin composition is improved, whereby the light resistance or the optical performance may be improved.

### (Addition Amount of Hindered Amine-Based Compound)

The addition amount of the hindered amine compound used in the present invention is from 0.05 parts by mass to 5 parts by mass, preferably from 0.1 to 4 parts by mass, and more preferably from 0.2 to 3 parts by mass, based on 100 parts by mass of the polymer having an alicyclic structure.

If the addition amount of the hindered amine compound is no less than the lower limit, the density of the functional groups of the hindered amine compound becomes sufficient, and as a result, the light resistance is sufficiently exhibited. Thus, the change in the shapes and the refractive index during the use is suppressed, and further, the generation of the microcracks can be suppressed. On the other hand, if the addition amount of the hindered amine compound is no more than the upper limit, the hindered amine compound can be uniformly dispersed in the resin composition, thereby ensuring the transparency of the molded product. That is, by using the hindered amine compound within the above value range, good light resistance can be achieved while not impairing the transparency, or the like of the molded product.

### (Process for Preparing Hindered Amine-Based Compound)

As the hindered amine compound used in the present invention, for example, the compound represented by General Formula (1) can be produced, for example, by suitably selecting the condition in accordance with the method as described JP-A-52-73886, 63-286448, 5-9356, 5-43745, and the like.

### [Piperidine Derivative and Salt Thereof]

The novel piperidine derivative having a piperidylaminotriazine skeleton of the present invention is represented by the following General Formula (20).

Following General Formula (20)

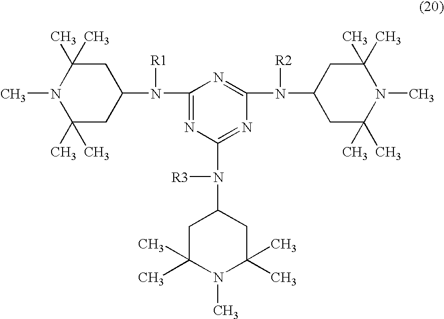

wherein R1 to R3 may be the same as or different from each other, and each represent an alkyl group having 1 to 18 carbon atoms.

A molded product comprising a resin composition containing the piperidine derivatives having a piperidylaminotriazine skeleton or a salt thereof has excellent weather resistance. The piperidine derivative or a salt thereof of the present invention has high compatibility with the above-described “polymer having an alicyclic structure at least in a part of a repeating structural unit”, and can provide a molded product comprising the resin composition containing the polymer with particularly excellent light resistance.

In General Formula (20), the alkyl group may be straight or branched, and examples thereof include a methyl group, an ethyl group, a propyl group, an isopropyl group, a butyl group, an isobutyl group, a sec-butyl group, a tert-butyl group, a hexyl group, a heptyl group, an octyl group, a nonyl group, a dodecyl group, and the like.

In the above General Formula (20), R1 to R3 are preferably all the same, and R1 to R3 are each more preferably an alkyl group having 4 to 12 carbon atoms.

Here, the alkyl group having 4 to 12 carbon atoms is most preferably a dodecyl group.

Examples of the piperidine derivative represented by the above General Formula (20) include the compounds represented by the above Chemical Formulae [31], [33], [35], and [42].

Examples of the salt of the compound represented by General Formula (20) of the present invention include salts with inorganic acids or organic acids. In this case, examples of the inorganic acids include hydrochloric acid, hydrobromic acid, hydroiodic acid, sulfuric acid, carbonic acid, and phosphoric acid. Further, examples of the organic acids include either optically active organic acids or optically non-active organic acids, for example, carboxylic acids such as formic acid, acetic acid, propionic acid, benzoic acid, trifluoroacetic acid, tartaric acid, and mandelic acid; sulfonic acids such as methanesulfonic acid, ethanesulfonic acid, benzenesulfonic acid, and p-toluenesulfonic acid, amino acids, and derivatives thereof. For the salts, the compositional ratio of the compound of the present invention and the acid may be equivalent or an any arbitrary ratio.

If the piperidine derivative or a salt thereof of the present invention is used as the hindered amine compound, the ratio of carbon atoms contained in the molecule structure can be from 67% by weight to 80% by weight, preferably 68% by weight to 79% by weight, and more preferably 70% by weight to 77% by weight.

If the ratio of carbon atoms of the piperidine derivative or a salt thereof is no less than the lower limit, a resin composition in which the piperidine derivative or a salt thereof is sufficiently dispersed can be obtained. As a result, in a molded product such as an optical component obtained from the resin composition, the light resistance can be sufficiently exhibited, and accordingly, the change in the shapes and the refractive index during the use is suppressed, and further, the generation of the microcracks can be suppressed. On the other hand, if the ratio of carbon atoms of the piperidine derivative or a salt thereof is no more than the upper limit, the density of the functional groups of the piperidine derivative or a salt thereof in the resin composition becomes sufficient, and as a result, more excellent light resistance can be exhibited. That is, by using the ratio of carbon atoms in the molecule structure within the above value range, a molded product such as an optical component having excellent light resistance can be obtained. As a result, in the use as an optical component, the deterioration of the optical characteristics can be suppressed, and in particular, in the use of a blue-violet laser light source, the deterioration of the optical characteristics can be effectively suppressed.

Further, the ratio of carbon atoms contained in the molecule structure as described above is a theoretical value as calculated from the chemical formula, but this theoretical value is substantially consistent to the ratio of carbon atoms as measured by CHN elemental analyzer (for example, CHNS-932 manufactured by LECO Corporation).

Also, the molecular weight of the piperidine derivative or a salt thereof used in the present invention can be from 500 to 3500, preferably from 600 to 3000, and more preferably from 700 to 2000.

If the molecular weight of the piperidine derivative or a salt thereof is no less than the lower limit, the movement of the piperidine derivative or a salt thereof is suppressed in the resin after molding. As a result, the light resistance is sufficiently exhibited, and accordingly, the change in the shapes and the refractive index during the use is suppressed, and accordingly, the generation of microcracks can be suppressed. On the other hand, if the molecular weight of the piperidine derivative or a salt thereof is no more than the upper limit, the fluidity upon melting becomes sufficient, and can be uniformly dispersed in the resin. As a result, the light resistance is sufficiently exhibited, and accordingly, the change in the shapes and the refractive index during the use is suppressed, and accordingly, the generation of microcracks can be suppressed. That is, by using the molecular weight of the piperidine derivative or a salt thereof within the above value range, as a result, both of at the molding and after the molding, the dispersibility of the piperidine derivative or a salt thereof is excellent, and accordingly, the change in the shapes and the refractive index of the molded product during the use is effectively suppressed, and accordingly, the generation of microcracks can be effectively prevented.

In addition, the molecular weight of the piperidine derivative or a salt thereof is a theoretical value as calculated from the chemical formula, but this theoretical value is substantially consistent to a weight average molecular weight in terms of polystyrene as measured by gel permeation chromatography (GPC), or a molecular weight as measured by mass analysis.

According to the piperidine derivative or a salt thereof used in the present invention, both of the ratio of carbon atoms and the molecular weight are within the above value range, and thus by using the compound in a predetermined amount, the dispersibility in the resin composition can be improved, and a molded product having excellent light resistance, transparency, or the like, and suppressed deterioration of the optical characteristics during the use of a blue-violet laser light source can be obtained.

In the resin composition of the present invention, by using the piperidine derivative or a salt thereof and the polymer represented by General Formula (3), an optical component having low reduction in the light transmittance during the use of a blue-violet laser light source and low deterioration in optical performances while maintaining moldability, low birefringence, heat resistance, mass productivity, mechanical strength, and light transmittance can be obtained. The optical component and the optical pickup device comprising the resin composition have sufficient optical performance, hardly deteriorate with a laser light close to ultraviolet ray, and hardly change the performances during the use, whereby it has industrially high value.

### (Addition Amount of Piperidine Derivative or Salt Thereof)

The addition amount of the piperidine derivative or a salt thereof used in the present invention is from 0.05 parts by mass to 5 parts by mass, preferably from 0.1 part by mass to 4 parts by mass, and more preferably from 0.2 part by mass to 3 parts by mass, based on 100 parts by mass of a polymer having an alicyclic structure.

If the addition amount of the piperidine derivative or a salt thereof is no less than the lower limit, the density of the functional groups of the piperidine derivative or a salt thereof becomes sufficient, and as a result, the light resistance is sufficiently exhibited. Accordingly, the change in the shapes and the refractive index during the use is suppressed, and further, the generation of the microcracks can be suppressed. On the other hand, if the addition amount of the piperidine derivative or a salt thereof is no more than the upper limit, the piperidine derivative or a salt thereof can be uniformly dispersed in the resin composition, thereby ensuring the transparency of the molded product. That is, by using the piperidine derivative or a salt thereof within the above value range, good light resistance can be achieved while not impairing the transparency, or the like of the molded product.

### [Process for Preparing Piperidine Derivative]

The piperidine derivative represented by General Formula (20) of the present invention can be obtained by reacting a compound represented by the following General Formula (21) with a chlorotriazine represented by the following General Formula (22).

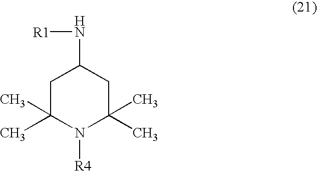

(wherein R1 represents an alkyl group having 1 to 18 carbon atoms, and R4 represents a hydrogen atom or a methyl group).

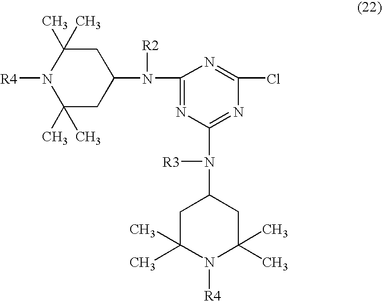

(wherein R2 and R3 may be the same as or different from each other, and each represent an alkyl group having 1 to 18 carbon atoms, and R4 represents a hydrogen atom or a methyl group).

Moreover, in a case where R4 of the compounds represented by General Formulae (21) and (22) is a hydrogen atom, the piperidine derivative is obtained by conversion into a methyl group by an Eschweiler-Clarke reaction. Here, the Eschweiler-Clarke reaction refers to one kind of the Leuckart-Wallach reactions, in which an amine is methylated using formaldehyde.

The molar ratio of the compound represented by General Formula (21) to the compound represented by General Formula (22) is most preferably 1:1, but either compound may be supplied in excess quantity. When the excess quantity is used, the amount is 1.01 to 10.0-fold, based on the preferable amount. The method for feeding both compounds to the reactor vessel is not particularly limited, for example, the total amount of both compounds may be together transferred to the reactor vessel to start the reaction, or the one compound may be gradually added to the other compound while being reacting.

The reaction may be carried out in the presence of a deoxidizing agent. Examples of the deoxidizing agent to be used include an inorganic salt such as sodium hydroxide, potassium hydroxide, sodium carbonate, potassium carbonate, and the like; and an organic salt such as triethylamine, tributylamine, pyridine, N,N-dimethylaniline, and the like.

The solvent used for the reaction is not particularly limited, unless the solvent effects the reaction, and examples thereof include water; saturated hydrocarbons such as pentane, hexane, heptane, cyclohexane, and the like; aromatic hydrocarbons such as benzene, toluene, xylene, and the like; halogenated hydrocarbons such as dichloromethane, chloroform, carbon tetrachloride, dichloroethane, chlorobenzene, dichlorobenzene, and the like; ethers such as ethylene glycol dimethyl ether, 1,3-dioxane, 1,4-dioxane, tetrahydrofuran, dimethyl ether, diethyl ether, diisopropyl ether, dibutyl ether, and the like; amides such as N,N-dimethylacetamide, and the like; nitriles such as acetonitrile, and the like; ketones such as acetone, methyl ethyl ketone, methyl isobutyl ketone, and the like; esters such as methyl acetate, ethyl acetate, and the like; sulfur-containing solvents such as dimethyl sulfoxide, and the like; 1,3-dimethyl-2-imidazolidinone (DMI); and the like. These solvents may be used singly or as a mixture, and when the solvent is used as a mixture, the solvent may use in any ratio.

The reaction is carried out at a temperature in a range from 0° C. to 300° C., and preferably 0° C. to 250° C. If the upper limit thereof is set according to the boiling point of the solvent to be used, the reaction may be carried out in an autoclave.

The isolation method for the piperidine derivative represented by General Formula (20) of the present invention is not particularly limited. When the product is deposited from the reaction solvent, isolation is possible by a filtration or centrifugation. When the product is dissolved in the reaction solvent, the method for distilling off the solvent under reduced pressure, or the method including adding a suitable solvent to deposit the product, and then filtrating or centrifuging the product, may be adapted. Alternatively, the product may be treated with suitable acid to form salt, and then the above procedure may be carried out, and these processes may be carried out in the combination.

If it is necessary to purify the compound represented by General Formula (1) of the present invention, a method known as a routine method can be employed, and examples thereof include methods of recrystallization, column chromatography, washing (sludge method) by a solvent, and treatment with activated carbon. The purification of these may be carried out after treating the compound represented by General Formula (1) with a suitable acid to form a salt.

The solvent used for purification is not particularly limited, and examples thereof include water; saturated hydrocarbons such as pentane, hexane, heptane, cyclohexane, and the like; aromatic hydrocarbons such as benzene, toluene, xylene, and the like; halogenated hydrocarbons such as dichloromethane, chloroform, carbon tetrachloride, dichloroethane, chlorobenzene, dichlorobenzene, and the like; ethers such as ethylene glycol dimethyl ether, 1,3-dioxane, 1,4-dioxane, tetrahydrofuran, dimethyl ether, diethyl ether, diisopropyl ether, dibutyl ether, and the like; amides such as N,N-dimethylacetamide, and the like; nitriles such as acetonitrile, and the like; ketones such as acetone, methyl ethyl ketone, methyl isobutyl ketone, and the like; esters such as methyl acetate, ethyl acetate, and the like; sulfur-containing solvents such as dimethyl sulfoxide, and the like; 1,3-dimethyl-2-imidazolidinone (DMI); and the like. These solvents may be used singly or as a mixture, and when the solvent is used as the mixture, the solvent may use in any ratio.

Further, the compound represented by General Formula (20) of the present invention can be obtained by reacting a compound represented by the following General Formula (21) and a cyanuric halide such as cyanuric chloride at a ratio of 3:1.

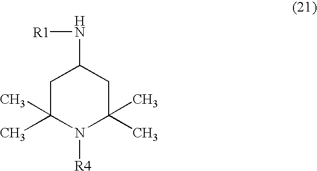

(wherein R1 represents an alkyl group having 1 to 18 carbon atoms, and R4 represents a hydrogen atom or a methyl group).

Moreover, if R4 in the compound represented by General Formula (21) is a hydrogen atom, the compound can be obtained by an Eschweiler-Clarke reaction for conversion into a methyl group.

This reaction is carried out under the same condition as in the reaction as described above of the compound represented by General Formula (21) with a compound represented by General Formula (22).

The compound represented by General Formula (21) of the present invention can be suitably prepared by the reaction of 2,2,6,6-tetramethyl-4-piperidone and alkylamine in the presence of hydrogen, and a hydrogenation catalyst.

At this time, examples of the reaction solvent include water; alcohols such as methanol, ethanol, isopropanol, and the like; saturated hydrocarbons such as pentane, hexane, heptane, cyclohexane, and the like; aromatic hydrocarbons such as benzene, toluene, xylene, and the like; and the like. These solvents may be used singly or as a mixture, and when the solvent is used as the mixture, the solvent may use in any ratio. The reaction can be carried out without a solvent. The amount of the solvent to be used is not particularly limited, but it is 0 to 100-fold by weight, and preferably 0 to 50-fold by weight, based on the amount of the starting materials, considering capacity efficiency and stirring efficiency.

The reaction temperature is from 10° C. to 100° C., and preferably from 20° C. to 80° C. The hydrogen pressure is from 0.01 MPa to 1 MPa, and preferably from 0.1 MPa to 0.5 MPa.

As the catalyst, for example, platinum or palladium, and preferably platinum can be used. This catalyst can be used as not supported or supported on a suitable inert material, such as carbon, calcium carbonate, alumina, and the like.

The amount of the alkylamine to be used to 2,2,6,6-tetramethyl-4-piperidone is from 0.8-fold mol to 1.5-fold mol, and preferably from 0.9-fold mol to 1.1-fold mol.

After completion of the reaction, the catalyst is separated by filtration, and then subjected to desolventation or distillation of a product for the use in the next process.

### [Phosphorus Stabilizer]

The resin composition of the present invention can comprise preferably 0.01 to 1 parts by mass, more preferably 0.02 to 0.8 parts by mass, and particularly preferably 0.05 to 0.6 parts by mass of the phosphorus stabilizer, based on 100 parts by mass of the polymer having an alicyclic structure in at least a part of repeating structural unit.

If the content of the phosphorus stabilizer is no less than the lower limit, the density of the functional groups of the phosphorus stabilizer becomes sufficient in the resin composition. As a result, the resulting molded product sufficiently exhibits light resistance, and accordingly the change in the shapes is suppressed during the use, and further the generation of microcracks can be prevented. On the other hand, the content of the phosphorus stabilizer is no more than the upper limit, the stabilizer is uniformly dispersed in the resin, ensuring the transparency, and thus the change in the refractive index during the use is suppressed. That is, when the content of the phosphorus stabilizer is within the above range, the change in the shapes of the molded product during the use is suppressed, and further the generation of microcracks can be prevented, and further, transparency is ensured, and thus the change in the refractive index during the use is suppressed.

As the phosphorus stabilizer used in the present invention, a compound having a phosphoric ester structure and a phenol structure in one molecule can be employed. By using a phosphorus stabilizer having such a structure, coloration of a molded product can be suppressed, and thus a stable light transmittance can be obtained either during the preparation, and during the use.

As the phosphorus stabilizer having a phosphoric ester structure and a phenol structure in one molecule, a compound represented by the following General Formula (5) can be used.

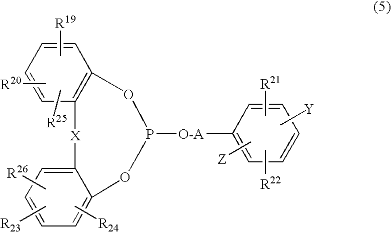

In General Formula (5), R¹⁹ to R²⁴ each independently represent a hydrogen atom, an alkyl group having 1 to 8 carbon atoms, a cycloalkyl group having 5 to 8 carbon atoms, an alkyl cycloalkyl group having 6 to 12 carbon atoms, an aralkyl group having 7 to 12 carbon atoms, or a phenyl group, and R²⁵ to R²⁶ each independently represent a hydrogen atom or an alkyl group having 1 to 8 carbon atoms.

X represents a single bond, a sulfur atom, or a —CHR²⁷— group (wherein R²⁷ represents a hydrogen atom, an alkyl group having 1 to 8 carbon atoms, or a cycloalkyl group having 5 to 8 carbon atoms).

A represents an alkylene group having 2 to 8 carbon atoms or a *—COR²⁸— group (wherein R²⁸ represents a single bond or an alkylene group having 1 to 8 carbon atoms, and * represents bonding to an oxygen atom).

One of Y and Z represents a hydroxyl group, an alkoxy group having 1 to 8 carbon atoms, or an aralkyloxy group having 7 to 12 carbon atoms, and the other represents a hydrogen atom or an alkyl group having 1 to 8 carbon atoms.

By using this phosphorus stabilizer represented by General Formula (5), the coloration of the molded product can be effectively suppressed, and thus a stable light transmittance can be obtained either of during the preparation and during the use.

As the phosphorus represented by General Formula (5), Sumilizer GP (trade name, manufactured by Sumitomo Chemical Co., Ltd.) can be used.

Further, as the phosphorus stabilizer used in the present invention, a phosphorus stabilizer having a saturated alkyl chain structure having 6 or more carbon atoms can be used. By using the phosphorus stabilizer having this structure, the dispersibility in the resin is improved, and the transparency is ensured. Thus, the change in the shapes during the use is suppressed, and further, the generation of microcracks or the change in the refractive index is also suppressed.

As the phosphorus stabilizer having a saturated alkyl chain structure having 6 or more carbon atoms, a compound represented by the following General Formula (6) can be used.

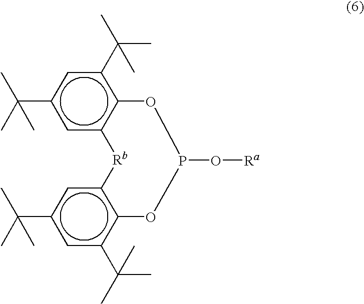

(wherein R^{a} represents an alkyl group having 1 to 24 carbon atoms, and preferably 6 to 24 carbon atoms, and R^{b} represents a single bond, a sulfur atom, or a —CHR^{c}— group (wherein R^{c} represents a hydrogen atom, an alkyl group having 1 to 8 carbon atoms, or a cycloalkyl group having 5 to 8 carbon atoms).

As the phosphorus stabilizer represented by General Formula (6), ADKSTAB HP-10 (trade name, manufactured by ADEKA Corporation) and the like can be used.

### [Hydrophilic Stabilizer]

The resin composition of the present invention preferably comprises 0.05 to 5 parts by mass of hydrophilic stabilizer based on 100 parts by mass of the polymer having an alicyclic structure at least in a part of a repeating structural unit, for the purpose of improving the humidity and thermal resistance property of the resin and improving the releasing property at the time of molding. Examples of the hydrophilic stabilizer include a compound containing, for example, a polyvalent alcohol described in JP-A-09-241484, a polyvalent alcohol, an ester of a polyvalent alcohol and a fatty acid, a sorbitol derivative, a compound having a hydrophilic group and a hydrophobic group described in JP-A-2001-26718, and the like.

### (Polyvalent Alcohol)

As such polyvalent alcohol, ones having the molecular weight of 2,000 or less and the ratio of carbon atom number to the number of a hydroxyl group in the same molecule of 1.5 to 30, preferably 3 to 20, particularly preferably 6 to 20 and having 6 or more carbon atoms, can be exemplified. Within the above range of the ratio and the carbon atom number, the compatibility with the thermoplastic resin is excellent, and an adverse effect on the transparency caused by forming foam at the time of melt-kneading is avoided. The range of the carbon atom number is preferably 6 to 100, and more preferably 6 to 60.

As the polyvalent alcohol, polyvalent alcohols in which at least one hydroxyl group in the molecule is bonded with a primary carbon atom, or in which the ratio of carbon atom number/hydroxyl group number is from 1.5 to 30, and in which the carbon atom number is 6 or more, are preferable.

Examples of the polyvalent alcohol of the present invention include a polyvalent alcohol containing an ether bond, a thioether bond, an alicyclic hydrocarbon group, or an aromatic hydrocarbon group in the molecule, but the aliphatic polyvalent alcohol is preferable.

Specific examples of the polyvalent alcohol include 3,7,11,15-tetramethyl-1,2,3-trihydroxyhexadecane, dihydroxyoctane, trihydroxyoctane, tetrahydroxyoctane, dihydroxynonane, trihydroxynonane, tetrahydroxynonane, pentahydroxynonane, hexahydroxynonane, dihydroxytriacontane, trihydroxytriacontane, eicosahydroxytriacontane, and the like. Among these, 3,7,11,15-tetramethyl-1,2,3-trihydroxyhexadecane is preferable.

Additionally, specific examples of the polyvalent alcohol also include 1,2-hexadecanediol, 2,3-heptadecanediol, 1,3-octadecanediol, 1,2-decyltetradecanediol, and the like.

### (Ester of Polyvalent Alcohol and Fatty Acid)

As the ester of the polyvalent alcohol and the fatty acid, for example, a sorbitol derivative, and the like disclosed in JP-A-2001-26682 are preferably used as they are excellent in transparency and provide a resin composition giving a less deterioration of transparency under the conditions of high temperature and high humidity atmosphere.

Besides, partially esterified glycelin or pentaerythritol which is fatty acid ester of the polyvalent alcohol disclosed in JP-B-07-007529 is also included as the preferable examples.

### (Sorbitol-Based Derivative)

Examples of the sorbitol derivative used in the present invention include the compounds represented by the following General Formulae (14) to (19)

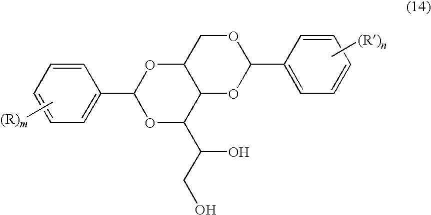

In Formula (14), each of R and R′ may be the same as or different from each other, and is any one of an alkyl group having 1 to 8 carbon atoms, a halogen atom, and an alkoxy group having 1 to 4 carbon atoms, and m and n are each independently an integer of 0 to 3.

Specific examples of the compound represented by above Formula (14) include 1,3,2,4-dibenzylidensorbitol, 1,3-benzyliden-2,4-p-methylbenzylidensorbitol, 1,3-benzyliden-2,4-p-ethylbenzylidensorbitol, 1,3-p-methylbenzyliden-2,4-benzylidensorbitol, 1,3-p-ethylbenzyliden-2,4-benzylidensorbitol, 1,3-p-methylbenzyliden-2,4-p-ethylbenzylidensorbitol, 1,3-p-ethylbenzyliden-2,4-p-methylbenzylidensorbitol, 1,3,2,4-di(p-methylbenzyliden)sorbitol, 1,3,2,4-di(p-ethylbenzyliden)sorbitol, 1,3,2,4-di(p-n-propylbenzyliden)sorbitol, 1,3,2,4-di(p-i-propylbenzyliden)sorbitol, 1,3,2,4-di(p-n-butylbenzyliden)sorbitol, 1,3,2,4-di(p-s-butylbenzyliden)sorbitol, 1,3,2,4-di(p-t-butylbenzyliden)sorbitol, 1,3,2,4-di(2′,4′-dimethylbenzyliden)sorbitol, 1,3,2,4-di(p-methoxybenzyliden)sorbitol, 1,3,2,4-di(p-ethoxybenzyliden)sorbitol, 1,3-benzyliden-2,4-p-chlorbenzylidensorbitol, 1,3-p-chlorbenzyliden-2,4-benzylidensorbitol, 1,3-p-chlorbenzyliden-2,4-p-methylbenzylidensorbitol, 1,3-p-chlorbenzyliden-2,4-p-ethylbenzylidensorbitol, 1,3-p-methylbenzyliden-2,4-p-chlorbenzylidensorbitol, 1,3-p-ethylbenzyliden-2,4-p-chlorbenzylidensorbitol, and 1,3,2,4-di(p-chlorbenzyliden)sorbitol, and a mixture of two or more of these, and particularly, 1,3,2,4-dibenzylidensorbitol, 1,3,2,4-di(p-methylbenzyliden)sorbitol, 1,3,2,4-di(p-ethylbenzyliden)sorbitol, 1,3-p-chlorbenzyliden-2,4-p-methylbenzylidensorbitol, 1,3,2,4-di(p-chlorbenzyliden)sorbitol, and a mixture of two or more of these can be preferably used.

Among the sorbitol derivatives described above, a compound represented by the following General Formula (15) can be mentioned as the preferable example.

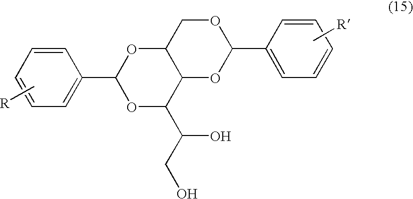

In Formula (15), R and R′ may be the same as or different from each other, and each represent a methyl group or an ethyl group.

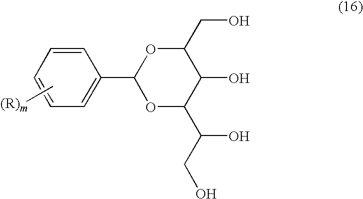

In Formula (16), each R may be the same as or different from each other, and is any one of an alkyl group having 1 to 8 carbon atoms, a halogen atom, and an alkoxy group having 1 to 4 carbon atoms, and m is an integer of 0 to 3.

Specifically, as the compound represented by the above General Formula (16), 2,4-benzylidensorbitol, 2,4-p-n-propylbenzylidensorbitol, 2,4-p-i-propylbenzylidensorbitol, 2,4-p-n-butylbenzylidensorbitol, 2,4-p-s-butylbenzylidensorbitol, 2,4-p-t-butylbenzylidensorbitol, 2,4-(2′,4′-dimethylbenzyliden) sorbitol, 2,4-p-methoxybenzylidensorbitol, 2,4-p-ethoxybenzylidensorbitol, 2,4-p-chlorbenzylidensorbitol, and a mixture of two or more of these can be used.

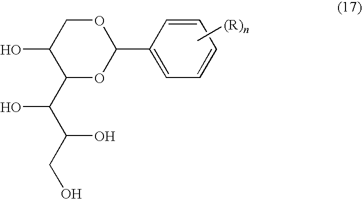

In Formula (17), each R may be same as or different from each other, and is any one of an alkyl group having 1 to 8 carbon atoms, a halogen atom, and an alkoxy group having 1 to 4 carbon atoms, and n is an integer of 0 to 3.

Specifically, as the compound represented by the above Formula (17), 1,3-benzylidensorbitol, 1,3-p-n-propylbenzylidensorbitol, 1,3-p-i-propylbenzylidensorbitol, 1,3-p-n-butylbenzylidensorbitol, 1,3-p-s-butylbenzylidensorbitol, 1,3-p-t-butylbenzylidensorbitol, 1,3-(2′,4′-dimethylbenzyliden)sorbitol, 1,3-p-methoxybenzylidensorbitol, 1,3-p-ethoxybenzylidensorbitol, 1,3-p-chlorbenzylidensorbitol, and a mixture of two or more of these can be used.

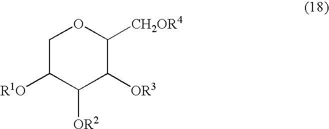

In Formula (18), R¹ to R⁴ are each an aliphatic acyl group having 10 to 30 carbon atoms or a hydrogen atom.

Specifically, as the compound represented by the above General Formula (18) , 1,5-sorbitanmonostearate, 1,5-sorbitandistearate, 1,5-sorbitantristearate, 1,5-sorbitanmonolaurate, 1,5-sorbitandilaurate, 1,5-sorbitantrilaurate, 1,5-sorbitanmonopalmitate, 1,5-sorbitandipalmitate, 1,5-sorbitantripalmitate, and a mixture of two or more of these can be used.

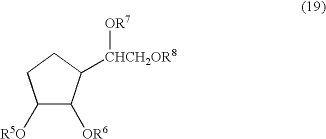

In Formula (19), R⁵ to R⁸ are each an aliphatic acyl group having 10 to 30 carbon atoms or a hydrogen atom.

Specifically, as the compound represented by Formula (19), 1,4-sorbitanmonostearate, 1,4-sorbitandistearate, 1,4-sorbitantristearate, 1,4-sorbitanmonolaurate, 1,4-sorbitandilaurate, 1,4-sorbitantrilaurate, 1,4-sorbitanmonopalmitate, 1,4-sorbitandipalmitate, and 1,4-sorbitantripalmitate, and a mixture of two or more of these can be used.

Among above sorbitol derivatives, benzylidenesorbitol derivatives represented by above Formulae (14) to (17) are preferable and dibenzylidenesorbitol derivative represented by above Formula (14) is more preferable. The sorbitol derivatives represented by Formulas (14) to (19) may be used singly or in combination with each other.

In the specification, in order to improve dispersibility of the sorbitol derivatives mentioned above, the derivatives may be mixed with fatty acids. An example of the fatty acids to be used includes an fatty acid having 10 to 30 carbon atoms.

### (Other Esters)

For the other polyvalent alcohol/fatty acid esters, ones in which a part of the alcoholic hydroxyl group is esterified can be used. A part of the specific examples of the polyvalent alcohol/fatty acid esters which can be used includes glycerin fatty acid esters such as glycerin monostearate, glycerin monolaurate, glycerin monomiristate, glycerin monopalmitate, glycerin distearate, glycerin dilaurate, and the like; pentaerythritol fatty acid esters such as pentaerythritol monostearate, pentaerythritol monolaurate, pentaerythritol distearate, and pentaerythritol dilaurate, pentaerythritol tristearate, and the like.

### (Compound Having Hydrophilic Group and Hydrophobic Group)

Examples of the compounds having a hydrophilic group and a hydrophobic group in the molecule include an amine compound or amide compound in which the hydrophilic group in the compound is a hydroxyl alkyl group and the hydrophobic group is an alkyl group having 6 or more carbon atoms.

Specifically, examples thereof include myristyl diethanolamine, 2-hydroxyethyl-2-hydroxydodecylamine, 2-hydroxyethyl-2-hydroxytridecylamine, 2-hydroxyethyl-2-hydroxytetradecylamine, pentaerythritol monostearate, pentaerythritol distearate, pentaerythritol tristearate, di-2-hydroxyethyl-2-hydroxydodecylamine, alkyl (carbon atoms 8 to 18) benzyldimethylammonium chloride, ethylenebisalkyl (carbon atoms 8 to 18) amide, stearyl diethanolamide, lauryl diethanolamide, myristyl diethanolamide, palmityl diethanolamide, and the like. Among these, the amine compound or the amide compound having a hydroxy alkyl group is preferably used.

The amount of the above-described hydrophilic stabilizer to be blended is preferably 0.0001 to 10 parts by mass, more preferably 0.05 to 5 parts by mass, and particularly preferably 0.1 to 3 parts by mass, based on 100 parts by mass of the polymer having an alicyclic structure used in the present invention. By using the hydrophilic stabilizer in the above amount, lower light transmittance in the change in temperatures or humidity, or the generation of fine cracks can be prevented, and accordingly, good optical performances of the polymer are not disturbed.

[Other Stabilizers]

For the resin composition used in the present invention, in addition to the above component, within the above range of not disturbing excellent properties of the optical component of the present invention, a well-known hydrophilic stabilizer, a weather resistance stabilizer, a heat resistance stabilizer, an antistatic agent, a flame retardant, a slipping agent, an antiblocking agent, an antifog additive, a lubricant, a natural oil, a synthetic oil, a wax, an organic or inorganic filler, and the like may be blended.

For example, as for the weathering stabilizer to be blended as an arbitrary component, ultraviolet absorbers such as a benzophenone compound, a benzotriazole compound, a nickel compound, and a hindered amine compound can be mentioned.

Specific examples of the benzotriazole ultraviolet absorbers include 2-(5-methyl-2-hydroxyphenyl)benzotriazole, 2,2-hydroxy-3,5-bis(α,α-dimethylbenzyl)phenyl, 2-(2′-hydroxy-5′-methyl-phenyl)benzotriazole, 2-(2′-hydroxy-3′,5′-di-t-butyl-phenyl)benzotriazole, 2-(2′-hydroxy-3′-t-butyl-5′-methyl-phenyl)-5-chlorobenzotriazole, 2-(2′-hydroxy-3′,5′-di-t-butyl-phenyl)-5-chlorobenzotriazole, 2-(2′-hydroxy-4′-n-octoxyphenyl)benzotriazole, and the like; benzotriazole derivatives such as Tinuvin 328 and Tinuvin PS (both manufactured by Chiba-Geigy Co., Ltd.), and SEESORB709(2-(2′-hydroxy-5′-t-octylphenyl)benzotriazole, manufactured by Shiraishi Calcium Kaisha, LTD.) which are commercially available.

Specific examples of the benzophenone ultraviolet absorbers include 2,4-dihydroxybenzophenone, 2-hydroxy-4-methoxybenzophenone, 2,2′-dihydroxy-4-methoxybenzophenone, 2,2′-dihydroxy-4,4′-dimethoxybenzophenone, 2,2′-dihydroxy-4,4′-dimethoxy-5-sulfobenzophenone, 2-hydroxy-4-methoxy-2′-carboxybenzophenone, 2-hydroxy-4-methoxy-5-sulfobenzophenone trihydrate, 2-hydroxy-4-n-octoxy benzophenone, 2-hydroxy-4-octadecyloxy benzophenone, 2-hydroxy-4-n-dodecyloxy-benzophenone, 2-hydroxy-4-benzyloxybenzophenone, 2,2′,4,4′-tetrahydroxy benzophenone, 2-hydroxy-4-dodecyloxy benzophenone, 2-hydroxy-4-(2-hydroxy-3-methacryloxy)propoxy benzophenone, and the like, Uvinul 490 (a mixture of 2,2′-dihydroxy-4,4′-dimethoxy benzophenone and other tetra-substituted benzophenone, manufactured by GAF Corporation), and Permyl B-100 (benzophenone compound, manufactured by Ferro Corporation).

Examples of the hindered amine compound include 2,2,6,6-tetramethyl-4-piperidylstearate, 1,2,2,6,6-pentamethyl-4-piperidylstearate, 2,2,6,6-tetramethyl-4-piperidylbenzoate, N-(2,2,6,6-tetramethyl-4-piperidyl)dodecyl imide succinate, 1-[(3,5-ditertiary butyl-4-hydroxyphenyl)propionyloxyethyl]-2,2,6,6-tetramethyl-4-piperidyl-(3,5-ditertiary butyl-4-hydroxyphenyl)propionate, bis(2,2,6,6-tetramethyl-4-piperidyl)sebacate, bis(1,2,2,6,6-pentamethyl-4-piperidyl)sebacate, bis(1,2,2,6,6-pentamethyl-4-piperidyl)-2-butyl-2-(3,5-ditertiary butyl-4-hydroxybenzyl)malonate, N,N′-bis(2,2,6,6-tetramethyl-4-piperidyl)hexamethylenediamine, tetrakis(2,2,6,6-tetramethyl-4-piperidyl)-1,2,3,4-butanetetracarboxylate, tetrakis(1,2,2,6,6-pentamethyl-4-piperidyl)-1,2,3,4-butanetetracarboxylate, bis(2,2,6,6-tetramethyl-4-piperidyl)di(tridecyl)-1,2,3,4-butanetetracarboxylate, bis(1,2,2,6,6-pentamethyl-4-piperidyl)di(tridecyl)-1,2,3,4-butanetetracarboxylate, 3,9-bis[1,1-dimethyl-2-{tris(2,2,6,6-tetramethyl-4-piperidyloxycarbonyloxy)butylcarbonyloxy}ethyl]-2,4,8,10-tetraoxaspiro[5.5]undecane, 3,9-bis[1,1-dimethyl-2-{tris-(1,2,2,6,6-pentamethyl-4-piperidyloxycarbonyloxy)butylcarbonyloxy}ethyl]-2,4,8,10-tetraoxaspiro[5.5]undecane, 1,5,8,12-tetrakis[4,6-bis{N-(2,2,6,6-tetramethyl-4-piperidyl)butylamino}-1,3,5-triazin-2-yl]-1,5,8,12-tetraazadodecane, 1-(2-hydroxyethyl)-2,2,6,6-tetramethyl-4-piperidinol/dimethylsuccinate condensate, 2-tertiaryoctylamino-4,6-dichloro-s-triazine/N,N′-bis(2,2,6,6-tetramethyl-4-piperidyl)hexamethylenediamine condensate, N,N′-bis(2,2,6,6-tetramethyl-4-piperidyl)hexamethylenediamine/dibromoethane condensate, 2,2,6,6-tetramethyl-4-hydroxypiperidine-N-oxyl, bis(2,2,6,6-tetramethyl-N-oxylpiperidine)sebacate, tetrakis(2,2,6,6-tetramethyl-N-oxylpiperidyl)butane-1,2,3,4-tetracarboxylate, 3,9-bis(1,1-dimethyl-2-(tris(2,2,6,6-tetramethyl-N-oxylpiperidyl-4-oxycarbonyl)butylcarbonyloxy)ethyl)-2,4,6,10-tetraoxaspiro[5.5]undecane, 1,6-bis(2,2,6,6-tetramethyl-4-piperidylamino)hexane/dibromoethane polycondensate, 1,6-bis(2,2,6,6-tetramethyl-4-piperidylamino)hexane/2,4-dichloro-6-tertiaryoctylamino-s-triazine polycondensate, 1,6-bis(2,2,6,6-tetramethyl-4-piperidylamino)hexane/2,4-dichloro-6-morpholino-s-triazine polycondensate, and the like.

Further, examples of the heat-resistant stabilizer to be blended as an arbitrary component include phenol antioxidants such as tetrakis[methylene-3-(3,5-di-t-butyl-4-hydroxyphenyl)propionate] methane, alkyl ester of β-(3,5-di-t-butyl-4-hydroxyphenyl) propionic acid, and 2,2′-oxamidebis[ethyl-3-(3,5-di-t-butyl-4-hydroxyphenyl)propionate], fatty acid metal salts such as zinc stearate, calcium stearate, and calcium 1,2-dihydroxystearate, and polyalcohol fatty acid esters such as glycerin monostearate, glycerin distearate, pentaerythritol monostearate, pentaerythritol distearate, and pentaerythritol tristearate. Further, phosphorous stabilizers such as distearyl pentaerythritol diphosphite, phenyl-4,4′-isopropylidene diphenol-pentaerythritol diphosphite, bis(2,6-di-t-butyl-4-methylphenyl)pentaerythritol diphosphite, and tris(2,4-di-t-butylphenyl)phosphite, may be used.

These stabilizers may be blended singly or in combination. For example, blending of tetrakis[methylene-3-(3,5-di-t-butyl-4-hydroxyphenyl)propionate] methane, and zinc stearate and glycerin monostearate may be exemplified. Theses stabilizers may be used by blending one kind or two or more kinds thereof.

Further, examples of the process antioxidant include a phenol-containing antioxidant, a phosphoric-containing antioxidant, a sulfur-containing antioxidant, or the like. Among these, a phenol-containing antioxidant is preferred, and an alkyl substituted phenol-containing antioxidant is particularly preferred.

Examples of the phenol-containing antioxidant include an acrylate phenol compound such as 2-tertiarybutyl-6-(3-tertiarybutyl-2-hydroxy-5-methylbenzyl)-4-methylphenylacrylate, 2,4-di-tertiaryamyl-6-(1-(3,5-di-tertiaryamyl-2-hydroxyphenyl)ethyl)phenylacrylate described in JP-A-63-179953 and JP-A-1-168643; an alkyl-substituted phenol compound such as 2,6-di-tertiarybutyl-4-methylphenol, 2,6-di-tertiarybutyl-4-ethylphenol, octadecyl-3-(3,5-di-tertiarybutyl-4-hydroxyphenyl)propionate, 2,2′-methylene-bis(4-methyl-6-tertiarybutylphenol), 4,4′-butylidene-bis(6-tertiarybutyl-m-cresol), 4,4′-thiobis(3-methyl-6-tertiarybutylphenol), bis(3-cyclohexyl-2-hydroxy-5-methylphenyl)methane, 3,9-bis(2-(3-(3-tertiarybutyl-4-hydroxy-5-methylphenyl)propionyl oxy)-1,1-dimethylethyl)-2,4,8,10-tetraoxaspiro[5,5]undecane, 1,1,3-tris(2-methyl-4-hydroxy-5-tertiarybutylphenyl)butane, 1,3,5-trimethyl-2,4,6-tris(3,5-di-tertiarybutyl-4-hydroxybenzil) benzene, tetrakis(methylene-3-(3′,5′-di-tertiarybutyl-4′-hydroxyphenylpropionate)methane [that is, pentaerythrimethyl-tetrakis(3-(3,5-di-tertiarybutyl-4-hydroxyphenylpropionate)], triethyleneglycolbis(3-(3-tertiarybutyl-4-hydroxy-5-methylphenyl) propionate), tocophenol; a triazine group-containing phenol compound such as 6-(4-hydroxy-3,5-di-tertiarybutylanilino)-2,4-bisoctylthio-1,3,5-triazine, 6-(4-hydroxy-3,5-dimethylanilino)-2,4-bisoctylthio-1,3,5-triazine, 6-(4-hydroxy-3-methyl-5-tertiarybutylanilino)-2,4-bisoctylthio-1,3,5-triazine, and 2-octylthio-4,6-bis(3,5-di-tertiarybutyl-4-oxyanilino)-1,3,5-triazine. Among these, preferred are an acrylate phenol compound and alkyl-substituted phenol compound, more preferred is an alkyl-substituted phenol compound. In addition, tetrakis(methylene-3-(3′,5′-di-tertiarybutyl-4′-hydroxyphenylpropionate)methane is excellent in heat resistance and stability, and thus preferred.

A sulphur-containing antioxidant include, for example, dilauryl-3,3-thiodipropionate, dimyristyl-3,3′-thiodipropionate, distearyl-3,3-thiodipropionate, laurylstearyl-3,3-thiodipropionate, pentaerythritol-tetrakis-(β-lauryl-thio-propionate), and 3,9-bis(2-dodecylthioethyl)-2,4,8,10-tetraoxaspiro[5,5]undecane.

A lactone-containing antioxidant is not limited as long as it has a lactone structure in the compound, but an aromatic lactone compound is preferred. In particular, more preferred is the compound having a benzofuranone skeleton, still more preferred is 3-arylbenzofuran-2-one having an aryl group as a substituent in the side chain of a furan ring, and 5,7-di-tertiary-3-(3,4-dimethylphenyl)-3H-benzofuran-2-one may be exemplified.

### (Inorganic Dispersant/Inorganic Fine Particles)

A known inorganic dispersant can be added to the resin composition of the present invention. Further, according to the particle diameter of the inorganic dispersant, the transparency may be ensured.

As the inorganic dispersant establishing the transparency, the inorganic fine particles having an average particle diameter of 1 nm to 30 nm are preferable. The particle diameter of the inorganic fine particles is more preferably from 1 nm to 20 nm, and particularly preferably in a range from 1 nm to 10 nm. If the average particle diameter is 1 nm or more, the dispersibility of the inorganic fine particles becomes good, and accordingly, the optical performance can be ensured, whereas when the average particle diameter is 30 nm or less, the transparency of the obtained thermoplastic material composition can be ensured. Here, the average particle diameter refers to a diameter of an equivalent sphere having the same volume.

The ratio of the inorganic fine particles to the resin is not particularly limited, but it is preferably in a range of 70% by volume or less, and more preferably in a range of 50% by volume or less. With 70% by volume or less, the transparency of the obtained thermoplastic material composition can be ensured.

Further, the distribution of the particle diameters is not particularly limited, but in order to exhibit the effect of the present invention more efficiently, formation of a relatively narrower distribution is more suitably used rather than formation of broad distribution. Specifically, it is preferably in a range of the variation coefficient (value obtained by dividing a standard deviation by an average value, indicative of a difference in measured values, dimensionless number)±30, and more preferably in a range of the variation coefficient ±10.

Examples of the inorganic fine particles include oxide fine particles, sulfide fine particles, selenide fine particles, telluride fine particles, phosphide fine particles, double oxide fine particles, oxoate fine particles, double salt fine particles, and complex salt fine particles. Specific examples of the inorganic fine particles include those of titanium oxide, zinc oxide, aluminum oxide, zirconium oxide, hafnium oxide, niobium oxide, tantalum oxide, magnesium oxide, calcium oxide, strontium oxide, barium oxide, yttrium oxide, lanthanum oxide, cerium oxide, indium oxide, tin oxide, lead oxide, and double oxides containing these oxides, such as lithium niobate, potassium niobate, lithium tantalite, and the like, phosphates, sulfates formed by combination with these oxides, and other like, zinc sulfate, cadmium sulfate, zinc selenide, and cadmium selenide, but not limited to these.

As the inorganic fine particles, fine particles of semiconductor crystal composition can be suitably used. The semiconductor crystal composition is not particularly limited, but it is preferable that absorption, light emission, fluorescence, or the like does not occur in a wavelength region used for an optical device.

Specific examples of the composition include simple substances of the 14^{th} group elements in the periodic table such as carbon, silica, germanium and tin; simple substances of the 15^{th} group elements in the periodic table such as phosphor (black phosphor), simple substances of the 16^{th} group elements in the periodic table such as selenium and tellurium, compounds comprising a plural number of the 14^{th} group elements in the periodic table such as silicon carbide (SiC) compounds of an element of the 14^{th} group in the periodic table and an element of the 16^{th} group in the periodic table such as tin oxide (IV) (SnO₂), tin sulfide (II, IV) (Sn(II) Sn(IV) S₃), tin sulfide (IV) (SnS₂), tin sulfide (II) (SnS), tin selenide (II) (SnSe), tin telluride (II) (SnTe), lead sulfide (II) (PbS), lead selenide (II) (PbSe) and lead telluride (II) (PbTe), compounds of an element of the 13^{th} group in the periodic table and an element of the 15^{th} group in the periodic table (or III-V group compound semiconductors) such as boron nitride (BN), boron phosphide (BP), boron arsenide (BAs), aluminum nitride (AlN), aluminum phosphide (AlP) aluminum arsenide (AlAs), aluminum antimonide (AlSb), gallium nitride (GaN), gallium phosphide (GaP), gallium arsenide (GaAs), gallium antimonide (GaSb), indium nitride (InN), indium phosphide (InP), indium arsenide (InAs) and indium antimonide (InSb), compounds of an element of the 13^{th} group in the periodic table and an element of the 16^{th} group in the periodic table such as aluminum sulfide (Al₂S₃), aluminum selenide (Al₂Se₃), gallium sulfide (Ga₂S₃), gallium selenide (Ga₂Se₃) gallium telluride (Ga₂Te₃), indium oxide (In₂O₃), indium sulfide (In₂S₃), indium selenide (In₂Se₃) and indium telluride (In₂Te₃), compounds of an element of the 13^{th} group in the periodic table and an element of the 17^{th} group in the periodic table such as thalliumchloride (I) (TlCl), thallium bromide (I) (TlBr), thallium iodide (I) (TlI), compounds of an element of the 12^{th} group in the periodic table and an element of the 16^{th} group in the periodic table (or II-VI group compound semiconductors) such as zinc oxide (ZnO), zinc sulfide (ZnS), zinc selenide (ZnSe), zinc telluride (ZnTe), cadmium oxide (CdO), cadmium sulfide (CdS), cadmium selenide (CdSe), cadmium telluride (CdTe), mercury sulfide (HgS), mercury selenide (HgSe) and mercury telluride (HgTe), compounds of an element of the 15^{th} group in the periodic table and an element of the 16^{th} group in the periodic table such as arsenic sulfide (III) (As₂S₃), arsenic selenide (III) (As₂Se₃), arsenic telluride (III) (As₂Te₃), antimony sulfide (III) (Sb₂S₃), antimony selenide (III) (Sb₂Se₃), antimony telluride (III) (Sb₂Te₃), bismuth sulfide (III) (Bi₂S₃), bismuth selenide (III) (Bi₂Se₃) and bismuth telluride (III) (Bi₂Te₃), compounds of an element of the 11^{th} group in the periodic table and an element of the 16^{th} group in the periodic table such as copper oxide (I) (Cu₂O) and copper selenide (I) (Cu₂Se), compounds of an element of the 11^{th} group in the periodic table and an element of the 17^{th} group in the periodic table such as copper chloride (I) (CuCl), copper bromide (I) (CuBr), copper iodide (I) (CuI), silver chloride (AgCl) and silver bromide (AgBr), compounds of an element of the 10^{th} group in the periodic table and an element of the 16^{th} group in the periodic table such as nickel oxide (II) (NiO), compounds of an element of the 9^{th} group in the periodic table and an element of the 16^{th} group in the periodic table such as cobalt oxide (II) (CoO) and cobalt sulfide (II) (CoS), compounds of an element of the 8^{th} group in the periodic table and an element of the 16^{th} group in the periodic table such as triiron tetraoxide (Fe₃O₄) and iron sulfide (II) (FeS), compounds of an element of the 7^{th} group in the periodic table and an element of the 16^{th} group in the periodic table such as manganese oxide (II) (MnO), compounds of an element of the 6^{th} group in the periodic table and an element of the 16th group in the periodic table such as molybdenum sulfide (IV) (MOS₂) and tungsten oxide (IV) (WO₂), compounds of an element of the 5^{th} group in the periodic table and an element of the 16^{th} group in the periodic table such as vanadium oxide (II) (VO), vanadium oxide (IV) (VO₂) and tantalum oxide (V) (Ta₂O₅), compounds of an element of the 4^{th} group in the periodic table and an element of the 16^{th} group in the periodic table such as titanium oxide (such as TiO₂, Ti₂O₅, Ti₂O₃ and Ti₅O₉), compounds of an element of the 2^{th} group in the periodic table and an element of the 16^{th} group in the periodic table such as magnesium sulfide (MgS) and magnesium selenide (MgSe), chalcogen spinels such as cadmium oxide (II) chromium(III) (CdCr₂O₄), cadmiumselenide (II) chromium(III) (CdCr₂Se₄), coppersulfide (II) chromium(III) (CuCr₂S₄) and mercuryselenide (II) chromium (III) (HgCr₂Se₄), and barium titanate (BaTiO₃), and the like.

Further, the semiconductor clusters structures of which are established such as (BN)₇₅(BF₂)₁₅F₁₅ described in Adv. Mater., Vol. 4, p. 494 (1991) by G. Schmid, et al., and Cu₁₄₆Se₇₃ (triethylphosphine)₂₂ described in Angew. Chem. Int. Ed. Engl. Vol. 29 (1990), p. 1452 are also listed as examples.

Moreover, it is preferable that the inorganic fine particles have a small value of the linear expansion coefficient, since such a value can reduce the effect on the linear expansion coefficient of a composite by dispersion of the inorganic fine particles.

The above-described inorganic fine particles, for example, silicon nitride, and the like have strong covalent bonding property, and accordingly, they tend to have a low linear expansion coefficient. Thus, they can be suitably used. On the other hand, the linear expansion coefficient of the oxide crystal tens to be a little higher, but silicate, and the like have low linear expansion coefficients, and thus, they can be suitably used.

As these inorganic fine particles, one kind of the inorganic fine particles may be used, and also, several kinds of the inorganic fine particles may be used in combination. Several kinds of the inorganic fine particles may be in a mixed form, a core/shell (lamination) form, a compound form, a composite form in which another inorganic fine particle is present in one parent inorganic fine particle, and the like.

In a case where an inorganic dispersant is used in the present invention, modification for dispersing the inorganic dispersant can be carried out. The modification can be carried out for both of the resin and the inorganic dispersant, for the purpose of introduction of a polar group for improving the intermolecular force of the resin, or of inhibition of hydrogen bonding to prevent the aggregation of the inorganic dispersant.

As the modification method that can be carried out for the resin, a well known method is used, for example, including graft modifications of a polymer having an alicyclic structure.

As the modifiers, unsaturated carboxylic acids and the derivatives thereof are generally used. Specific examples of the unsaturated carboxylic acids include (meth) acrylic acid, maleic acid, fumaric acid, tetrahydrophthalic acid, itaconic acid, citraconic acid, crotonic acid, isocrotonic acid and endocisbicyclo[2,2,1]hept-5-ene-2,3-dicarboxylic acid (Nadic Acid™), and derivatives of the unsaturated carboxylic acids, for example, unsaturated carboxylic acid anhydrides, unsaturated carboxylic acid halides, unsaturated carboxylic acid amides, unsaturated carboxylic acid imides and ester compounds of the unsaturated carboxylic acids. More specific examples of the derivatives of the unsaturated carboxylic acid include maleic anhydride, citraconic anhydride, malenyl chloride, maleimide, monomethyl maleate, dimethyl maleate and glycidyl maleate.

Of the above modifiers, preferably used are α-β-unsaturated dicarboxylic acids and alpha, beta-unsaturated dicarboxylic acid anhydrides, e.g., maleic acid, nadic acid and anhydrides of these acids. The modifiers may be used in combination of two or more kinds thereof.

Further, as the modification method that can be carried out for the inorganic dispersant, a well known method is used. For example, by using a silane coupling agent, a silicone oil coupling agent, a titanate coupling agent, an aluminate coupling agent, a zirconate coupling agent, or the like as a surface-treatment agent, the surface treatment can be carried out. The surface modification methods are exemplified in, for example, JP-A-2006-273991, Patent Publication No. 2636204, and the like.

### [Process for Producing Resin Composition]

The process for preparing the resin composition of the present invention is not particularly limited, but the resin composition may be produced by the known methods. Specifically, pellet form resin composition can be obtained by adding the polymer having an alicyclic structure and the hindered amine compound, and the phosphorus stabilizer and the hydrophilic stabilizer depending on the purpose, furthermore, the other stabilizer within the above range which dose not lose the purpose of the present invention, being mixed and then flash dried, or mixing each component by the use of Henschel mixer, Ribon blender, melt blender, homomixer, or the like and then pelletized. Furthermore, the molded product can be obtained, in accordance with an injection molding process, an extrusion molding process, a blow molding process, a vacuum molding process, a slash molding process, depending on the shape of target molded product.

### (Content of Metal Component)

As for the resin composition of the present invention, the content of an iron atom (Fe) as the metal component contained in the resin composition is preferably 5 ppm or less, and more preferably 2 ppm or less. The content of the iron atom may be determined in accordance with a known process, including for example, an atomic absorption analysis, or the like. If the content of an iron atom is no more than the upper limit, the coloration of the molded product can be suppressed, thereby establishing the transparency.

### (Other Metal Component)

As for the other metal component, the content of the metal component which might lead the deterioration of the resin needs to be in the range which does not impair the effect of the present invention. Examples of the metal component include vanadium, zinc, and calcium. In the present invention, the metal which is mixed into the resin from a starting material, a catalyst, and a process, needs to be minimized. For example, a zinc compound such as zinc stearate used as a hydrochloric acid absorbent also has an effect leading the deterioration of the resin. In addition, when the content of the residual metal catalyst component in the resin is small, the optical properties such as transparency or the like is not disturbed, thus it being preferred.

### [Uses]

The molded product obtained from the resin composition of the present invention has excellent light resistance and transparency, and used in solar cells, sunshine roofs of automobiles, outdoor components of window frames, or the like, and optical components as described below.

### (Total Light Transmittance and Spectral Light Transmittance)

In a case of using the resin composition of the present invention for optical applications, it is necessary to transmit light beam. Therefore, it is preferable to have good light transmittance. The light transmittance is defined by a total light transmittance or a spectral light transmittance according to the applications.

In a case of using the resin composition in total light or a plurality of wavelength region, it is necessary to have good total light transmittance and the total light transmittance in a state where an antireflection coating is not provided on the surface is 85% or more, and preferably 88 to 93%. When the total light transmittance is 85% or more, the required amount of light can be obtained. A measuring method of the total light transmittance may use a well-known method and the measuring apparatuses are not limited. For example, on the basis of ASTM D1003, a method is exemplified by a method in which total light transmittance is obtained by molding the thermoplastic amorphous resin in such a way that a sheet having a thickness of 3 mm, and measuring the molded sheet using a haze meter.

Further, in a case of an optical system using only a specific wavelength, for example, a laser optical system, even when the total light transmittance is not relatively high, it can be still used as long as the spectral light transmittance in the wavelength is in a preferred range. In this case, the spectrum light transmittance in the using light wavelength in a state where the antireflection coating is not provided on the surface is preferably 85% or more, more preferably 86 to 93%. When the spectral light transmittance is 85% or more, the required amount of light can be obtained, thus it is preferable. As a measuring method and a measuring device, a well-known method and device can be used. Specifically, a spectral photometer may be exemplified as the measuring device.

Further, the molded product comprising the resin composition of the present invention has excellent light transmittance at a wavelength of 300 to 450 nm, as well as 390 to 420 nm, and particularly of 400 to 420 nm, for example, of laser light. The spectral light transmittance at a wavelength of 400 nm is 85% or more, and preferably 86 to 93%. In addition, the resin composition hardly generates deterioration, and thus, the optical property hardly changes when it is used as an optical component.

In addition, in a case of using in the optical components, a known antireflection coating can be provided so as to further improve the light transmittance.

### [Optical Component]

The molded product obtained from the resin composition of the present invention is excellent in the light transmittance at a wavelength in the range from 300 nm to 450 nm. Accordingly, the molded product may be used as the optical component in the optical system having a light source containing the wavelength in the range from 300 nm to 450 nm. The optical component is a component used for the optical machine, and specifically exemplified by an analytical cell used for a detector for UV, an optical component used for an imaging system using no UV cut filter, a filter for a solar battery, a sealant for LED, a lens used in an LED optical system, an optical component used in a light-emitting device such as an organic EL-related member, a lens for a projector, and a display panel, or the like.

The molded product obtained from the resin composition of the present invention may be also applied particularly suitably for an optical lens and an optical prism such as an imaging system lens of a camera; a lens such as a microscope, an endoscope, an telescope lens; a total light transmittance type lens such as an eyeglass lens; a pickup lens of an optical disk such as a CD, a CD-ROM, a WORM (a write once read many optical disk), an MO (a rewritable optical disk; a magneto optical disk), an MD (a mini disk), and a DVD (a digital video desk); a laser scanning lens such as an fθ lens of a laser beam printer and a lens for a censor; a prism lens of a finder system of a camera; a lens for a optical pickup device such as a sensor lens, a diffraction plate, a collimator, an objective lens, a beam expander, and a beam shaper; or the like. The molded product obtained from the resin composition of the present invention is particularly excellent in the light transmittance at a wavelength in the range from 390 to 420 nm, and thus may be suitably used as a lens for a optical pickup device using a blue-violet laser beam source. The optical disk application may be exemplified by a CD, a CD-ROM, a WORM (a write once read many optical disk), an MO (a rewritable optical disk; a magneto optical disk), an MD (a mini disk), and a DVD (a digital video desk), or the like. Examples of the other optical application include a light guide plate such as a liquid crystal display; an optical film such as a polarization film, a retardation film, and an optical diffusion film; an optical diffusion film; an optical card; and a liquid crystal display element substrate.

The resin composition of the present invention may be molded in a various form of spherical shape, rod-like shape, plate-like shape, column shape, cylindrical shape, tubular shape, fibrous shape, film shape, or sheet shape, and may be used in the various forms above.

The method of molding for obtaining an optical component is not particularly limited and a known method can be used. For the applications and shapes, although it is different in accordance with the applications and shapes, injection molding method, extrusion molding method, blow molding method, vacuum molding method, and slash molding method can be employed. However, from the viewpoints of moldability and productivity, the injection molding method is preferred. The molding condition is approximately selected according to a purpose of uses or the molding method, but the temperature of the resin in the injection molding method is generally selected from the range of 150 to 400° C., preferably 200 to 350° C., more preferably 230 to 330° C.

Since the resin composition of the present invention is excellent in low birefringence, transparency, mechanical strength, thermal resistance, and low absorption, it is possible to be used in various applications, and particularly it is possible to be used suitably in the optical component used in the optical pickup device.

[Optical Path Difference Providing Structure]

An optical path difference providing structure is a structure having a function of providing a predetermined optical path difference to a predetermined light on at least one optical surface of the optical components through which the light passes.

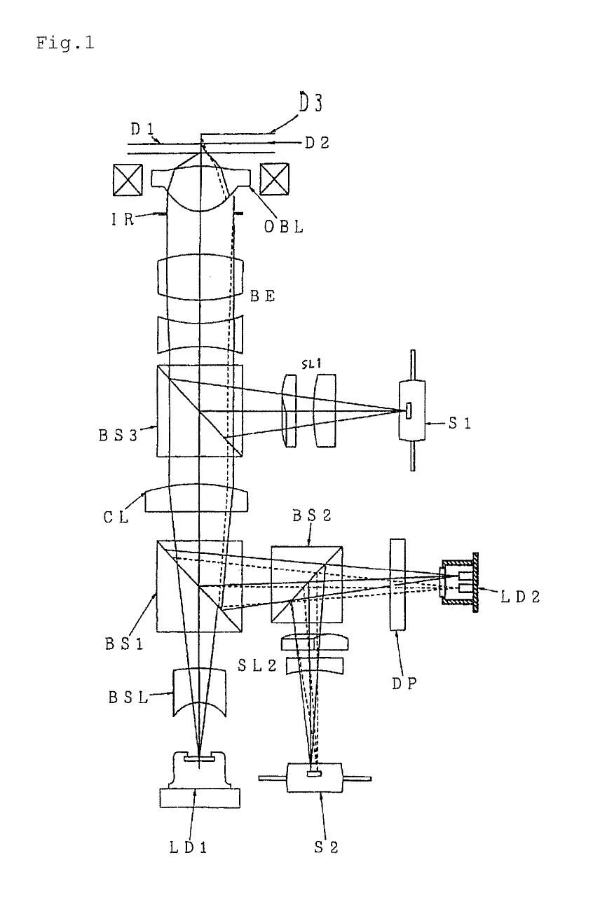

Hereinafter, it will be described in detail in FIG. 1 which relates to the pickup device.

The molded product obtained from the resin composition of the present invention is disposed in a common optical path of a first light source, a second light source, and a third light source and used in an objective lens OBL having a diffraction structure. Further, in the objective lens, a saw-like diffraction structure is provided.

This structure is provided in which fine steps are provided in a concentric pattern with a central focus on the optical axis, and the light beam passing through neighboring orbicular zones are given by the predetermined optical path difference. By setting a pitch (diffraction power) or a depth (brazed wavelength) of the saw structure, as for the ‘optical disc of high density’, the light beam from the first light source forms a light-collected spot by the second diffraction light, and as for the DVD, the light beam from the second light source forms a light-collected spot by a first diffraction light.

By using the light having a different diffraction order, an efficiency of diffraction in each case is improved so that the amount of light is secured.

As for the CD, it is preferable that the light beam from the third light source is set to a diffracted light having the order same to that of the DVD, but also may be set to the other suitable order. In this example, the first diffracted light is allowed as in the DVD to form a light-collected spot.

Such diffraction structure is one example of the optical path difference providing structure, and other known structures of ‘retardation providing structure’ or ‘multi level structure’ may also be employed.

Herein, the optical path difference providing structure is employed so as to correct a spherical aberration caused by the difference in thickness of the optical disc format, but it also can be used for correcting the aberration caused by the wavelength difference of the using wavelength or the variation in the using wavelength (mode hop). The former is the correction for a spherical chromatic aberration caused by the wavelength difference of 50 nanometer or more, and the latter is the correction for a small wavelength variation changing within 5 nm.

In this example, an example in which the diffraction structure is provided on the objective lens is described, but it is also possible to be provided on the other optical components such as a collimator or coupling lenses. It is most preferable to use such material in the optical component having a refracting surface and an aspherical surface. By using the resin composition of the present invention, prolonged use which is realized only in a glass in the past is now realized, and a lens having the optical path difference providing structure which is impossible in a glass lens can be easily provided.

[Optical Pickup Device]

An optical pickup device is a device having a function of playing back and/or recording information on an optical information recording medium, and which includes a light source for emitting light, and an optical component for irradiating light to the optical information recording medium and/or collecting light reflected from the optical information recording medium. Specifications of the device are not limited. However, in order to describe effects of the present invention, an example of an optical component used for the optical pickup device which can be obtained from the resin composition of the present invention will be described with reference to FIG. 1.

In FIG. 1, the target is the optical pickup device using the light source having the using wavelength of 405 nm, so-called blue-violet laser, which is 3-format compatible of ‘optical disc of high density’, DVD, and CD. The ‘optical disc of high density’ having the protective substrate thickness t1 of 0.6 mm is supposed as a first optical information medium, the DVD having the protective substrate thickness t2 of 0.6 mm is supposed as a second optical information recording medium, and the CD having the protective substrate thickness t3 of 1.2 mm is supposed as a third optical information recording medium. Each of D1, D2, and D2 represents the thickness of the substrate.

FIG. 1 is a schematic view showing an optical pickup device related to the present invention.

A laser diode LD1 is the first light source, and the blue-violet laser having a wavelength 2\1 of 405 nm is used but the laser having a wavelength in the range of 390 to 420 nm can be appropriately employed. LD2 is a second light source, and the red laser having a wavelength λ2 of 655 nm is used but the laser having a wavelength in the range of 630 to 680 nm can be appropriately employed. LD2 is also a third light source, and the infrared laser having a wavelength λ3 of 780 nm is used but the infrared laser having a wavelength in the range of 750 to 800 nm can be appropriately employed.

The laser diode LD2 is so-called light source unit of two-laser in one-package in which two light emitting points of the second light source (light source for DVD) and the third light source (light source for CD) are packed in a same package.

In this package, since the second light source is adjusted to be disposed on an optical axis, the third light source is disposed slightly away from the optical axis thereby resulting difference in an image height. Accordingly, techniques for improving this characteristic are already known so that such techniques can be employed if necessary. In the invention, a correcting plate DP is used to perform the correction. In the correcting plate DP, a grating is formed so that the displacement of the optical axes is corrected.

The solid line from LD2 is the light beam of light source for DVD, and the dashed line is the light beam of light source for CD. A beam splitter BS1 transmits or reflects the light beam of the light source entered from LD1 and LD2 in a direction towards the OBL of objective lens

In order to improve a beam quality, the light beam transmitted from the LD1 is entered to a beam shaper BSL, sent to the BS1 mentioned above, and then incident to the collimator CL thereby being collimated to infinite parallel light. Then, the light beam is sent to the beam splitter BS3, and then to the beam expander BE constituted by concave and convex lenses, and then entered to the objective lens OBL. Next, the light beam forms the light-collected spot on the information recording surface via a protective substrate of the first optical information recording medium. Further, the light beam is reflected on the information recording surface, passed the collimator CL via same path as above, a proceeding direction is converted by the beam splitter BS3, and then the light beam is collected to a sensor S1 via a sensor lens SL1. The light beam is subjected to a photoelectric conversion by the sensor thereby being converted into an electronic signal.

In addition, in-between the beam expander BE and objective lens OBL, a λ/4 (quarter the wavelength) plate not shown is disposed, such that gives a just half the wavelength change between the forwarding and returning process thus changing the polarization direction. Therefore, the proceeding direction of the light beam in the returning direction is changed by the BS3.

The beam shaper BSL has curvatures differing respectively for two directions of a direction perpendicular to the optical axis and a direction perpendicular to such direction (having a curvature of rotation asymmetric for the optical axis).

Each of the light beam emitted from the light source, under the semiconductor light source configuration, has a different divergence angle to two directions of a direction perpendicular to the optical axis and a direction perpendicular to such direction, and forms an elliptical shape as viewed in the optical axis direction, but it is not preferable for the light beam of the light source for the optical disc. Therefore, the light beam is subjected to different refractions in each direction by the beam shaper BSL so that the light beam emitted has an approximately circular cross section. In the invention, the beam shaper BSL is disposed in the optical path of LD1, but it can also be disposed in the optical path of LD2.

In the same manner as in LD1, the light beam transmitted from the LD2 forms a light-collected spot on an optical disc (a second optical information recording medium and a third optical information recording medium), reflects and then is finally collected in the sensor S2. Except that an agreement in the optical paths is made by the BS1, there is no change as compared to LD1.

The objective lens OBL is a single lens in this figure, but it may be formed of a plurality of optical components if necessary.

Since the resin composition of the present invention has low birefringence, it is obvious that the resin composition can be perfectly used in the device having such configuration.

[Actuator]

In FIG. 1 relating the optical pickup device, a state where the light beam transmitted from each LDs is collected on the information recording surface via a protective substrate of the optical disc is described, but a basic position is replaced by an actuator according to the optical disc for playing back/recording, and the focus slide (focusing) is performed from the reference position.

According to the thicknesses of a protective substrate and the size of a pit in each optical information recording medium, a numerical apertures required for the objective lens BL is changed. Here, the numerical apertures for CD is 0.45, and the numerical apertures for DVD and ‘optical disc of high density’ is 0.65, but those may be appropriately selected from the range of 0.43 to 0.5 as for the CD and from the range of 0.58 to 0.68 as for the DVD. IR is a diaphragm to cut unnecessary light.

The parallel light is incident on the objective lens OBL, but a configuration in which a collimation is not provided and a limited divergent light is incident may be employed.

By using the resin composition of the present invention, a long period of use realized only by a conventional method can be realized, and it is obvious that a torque required for an operation by the actuator or the like is significantly decreased as compared to the glass lens.

### EXAMPLES

Hereinafter, the present invention will be further explained in detail with the reference to Examples. Firstly, Synthesis Examples of the hindered amine compounds used in Examples will be described.

### (Hindered Amine Compound)

### Synthesis Example 1

### Synthesis of the exemplary compound 1 represented by Chemical Formula [13] (N,N′-dibutyl-,N″-lauryl-N,N′-bis-(1,2,2,6,6-pentamethyl-4-piperidin-4-yl)-[1,3,5]-triazine-2,4,6-triamine (LTABM))

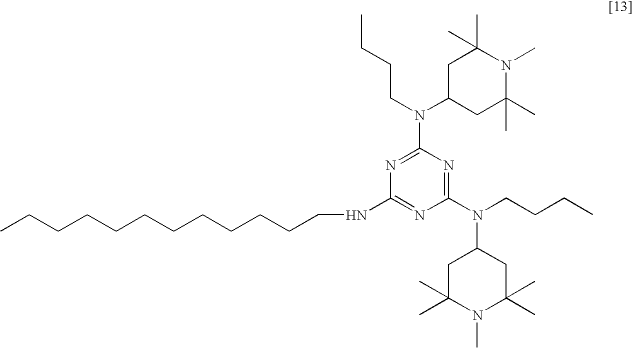

### (1) Synthesis of N-butyl-2,2,6,6-tetramethyl piperidine-4-amine (TABA)

108.67 g (0.7 mol) of 2,2,6,6-tetramethyl-4-piperidone (TAA), 53.76 g (0.735 g) of butylamine, and 2.52 g of 2% platinum carbon (50% water content) were charged into 163.0 g of methanol, and the mixture was allowed to undergo a reaction at a hydrogen pressure of 0.3 MPa and 50° C. for 2.5 hours. The catalyst was removed by filtration, and then desolvented, and distilled to obtain 127.93 g of a target compound as a colorless transparent liquid.

### (2) Synthesis of 2,4-bis(butyl(1,2,2,6,6-pentamethyl piperidin-4-yl)amino)-6-chloro-1,3,5-triazine (CTABM)

127.42 g (0.6 mol) of TABA and 27.5 g (0.66 mol) of 96% sodium hydroxide were charged into 175 g of water, and heated to a temperature of 60° C., and 55.32 g (0.3 mol) of cyanuric chloride dissolved in 210 g of toluene was then added dropwise thereto over 1 hour. Then, the mixture was aged at 65° C. to 78° C. for 3 hours, and the reaction mass was cooled, and washed with 100 g of water twice. 27.03 g (0.9 mol) of paraformaldehyde was charged into the reaction mass, and heated to a temperature of 80° C., and 30.38 g (0.66 mol) of formic acid was then added dropwise thereto over 1 hour. Then, the mixture was aged for 3 hours. The reaction mass was cooled, and then washed with 65 g of a 17% aqueous sodium hydroxide solution, and then twice with 100 g of water. The obtained toluene solution was desolvented, pulverized, and then dried to obtain 160.8 g of a target compound as a white crystal.

### (3) Synthesis of N,N′-dibutyl-, N″-lauryl-N,N′-bis-(1,2,2,6,6-pentamethyl-4-piperidin-4-yl)-[1,3,5]-triazine-2,4,6-triamine (LTABM)

54.83 g (0.10 mol) of CTABM and 8.29 g (0.06 mol) of potassium carbonate were charged into 60 g of dimethylacetamide, and heated to a temperature of 130° C., and then 18.54 g (0.10 mol) of laurylamine dissolved in 20 g of dimethylacetamide was then added dropwise thereto over 1 hour. Then, the mixture was aged at 130 to 145° C. for 2 hours. The reaction mass was cooled, and then discharged into 300 g of water, and the reaction product was extracted with 150 g of toluene and washed with 20 g of a 1 N aqueous sodium hydroxide solution and then twice with 100 g of water. The obtained toluene solution was purified by silica gel column chromatography, and concentrated to obtain 61.6 g of a target compound as a viscous liquid.

The exemplary compound 1 represented by Chemical Formula [13] has a theoretical molecular weight of 713.18, and a ratio of carbon atoms (theoretical value) of 72.42

### Synthesis Example 2

### Synthesis of the Exemplary Compound 2 Represented by Chemical Formula [20] (N,N′,N″-trilauryl-N,N′-bis-(2,2,6,6-tetramethyl piperidin-4-yl)-[1,3,5]-triazine-2,4,6-triamine (LTADA))

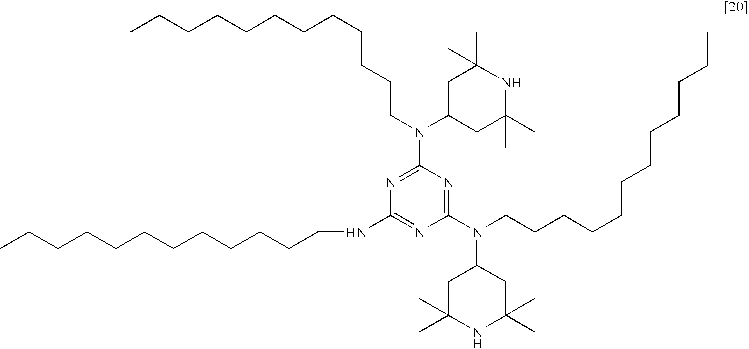

### (1) Synthesis of N-dodecyl-2,2,6,6-tetramethyl-2-piperidine-4-amine (TADA)

77.6 g (0.5 mol) of 2,2,6,6-tetramethyl-4-piperidone (TAA), 97.3 g (0.525 g) of laurylamine, and 1.8 g of 2% platinum carbon (50% water content) were charged into 116.4 g of methanol, and subjected to reaction at a hydrogen pressure of 0.3 MPa and 50° C. for 2.5 hours. The catalyst was removed by filtration, desolvented, and distilled to obtain 152.8 g of a target compound as a yellowish liquid.

### (2) Synthesis of 2,4-bis(dodecyl(2,2,6,6-tetramethylpiperidin-4-yl)amino)-6-chloro-1,3,5-triazine (CTADA)

110.0 g (0.339 mol) of TADA and 14.58 g (0.35 mol) of 96% sodium hydroxide were charged into 65 g of water, and heated to a temperature of 60° C., and 30.43 g (0.165 mol) of cyanuric chloride dissolved in 110 g of toluene was then added dropwise thereto over 1 hour. Then, the mixture was aged at 65° C. to 78° C. for 3 hours. The reaction mass was cooled, washed twice with 100 g of water, and desolvented to obtain 132.7 g of a target compound as a viscous liquid.

### (3) Synthesis of N,N′,N″-trilauryl-N,N′-bis-(2,2,6,6-tetramethyl piperidin-4-yl)-[1,3,5]-triazine-2,4,6-triamine (LTADA)

71.7 g (0.089 mol) of CTADA and 6.91 g (0.050 mol) of potassium carbonate were charged into 70 g of dimethylacetamide, and heated to a temperature of 130° C., and 16.51 g (0.089 mol) of laurylamine dissolved in 15 g of dimethylacetamide was then added dropwise thereto over 30 minutes. Then, the mixture was aged at 140° C. to 150° C. for 5 hours. The reaction mass was cooled, and then discharged into 300 g of water, and the reaction product was extracted with 150 g of toluene, and washed with 20 g of a 1 N aqueous sodium hydroxide solution, and again twice with 100 g of water. The obtained toluene solution was purified by silica gel column chromatography, and then concentrated to obtain 71.8 g of a target compound as a viscous liquid.

The exemplary compound 2 represented by Chemical Formula [20] has a theoretical molecular weight of 909.55, and a ratio of carbon atoms (theoretical value) of 75.27

### Synthesis Example 3

### Synthesis of the Exemplary Compound 3 Represented by Chemical Formula [34] (N,N′,N″-trilauryl-N,N′,N″-tris(2,2,6,6-tetramethyl-4-piperidin-4-yl)-[1,3,5]-triazine-2,4,6-triamine (TTADA))

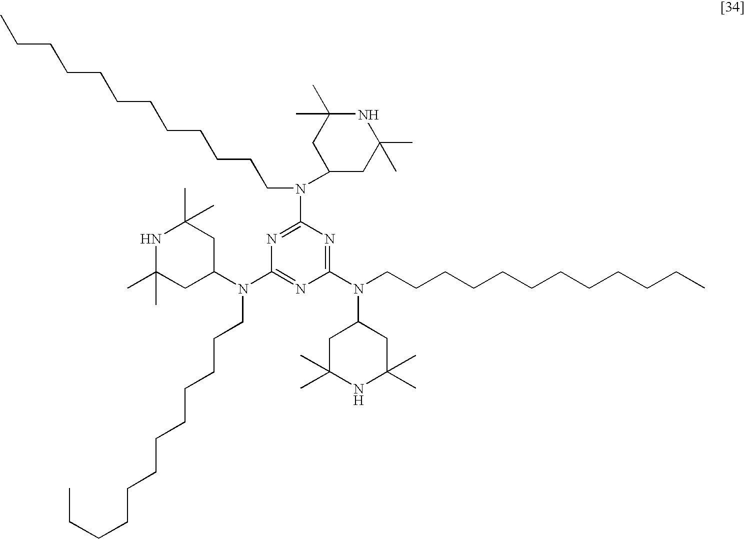

### (1) Synthesis of N-dodecyl-2,2,6,6-tetramethyl piperidine-4-amine (TADA)

77.6 g (0.5 mol) of 2,2,6,6-tetramethyl-4-piperidone (TAA), 97.3 g (0.525 g) of laurylamine, and 1.8 g of 2% platinum carbon (50% water content) were charged into 116.4 g of methanol, and the mixture was allowed to undergo a reaction at a hydrogen pressure of 0.3 MPa and 50° C. for 2.5 hours. The catalyst was removed by filtration, desolvented, and then distilled to obtain 152.8 g of a target compound as a yellowish liquid.

### (2) Synthesis of 2,4-bis(dodecyl(2,2,6,6-tetramethyl piperidin-4-yl)amino)-6-chloro-1,3,5-triazine (CTADA)

110.0 g (0.339 mol) of TADA and 14.58 g (0.35 mol) of 96% sodium hydroxide were charged into 65 g of water, and heated to a temperature of 60° C., and 30.43 g (0.165 mol) of cyanuric chloride dissolved in 110 g of toluene was then added dropwise thereto over 1 hour. Then, the mixture was aged at 65° C. to 78° C. for 3 hours. The reaction mass was cooled, washed twice with 100 g of water, and desolvented to obtain 132.7 g of a target compound as a viscous liquid.

### (3) Synthesis of N,N′,N″-trilauryl-N,N′,N″-tris(2,2,6,6-tetramethyl-4-piperidin-4-yl)-[1,3,5]-triazine-2,4,6-triamine (TTADA)

61.0 g (0.076 mol) of CTADA and 6.36 g (0.046 mol) of potassium carbonate were charged into 70 g of dimethylacetamide, and heated to a temperature of 130° C., and 24.64 g (0.076 mol) of TADA dissolved in 20 g of dimethylacetamide was then added dropwise thereto over 30 minutes. Then, the mixture was aged at 150° C. to 160° C. for 18 hours. The reaction mass was cooled, and then discharged into 300 g of water, and the reaction product was extracted with 150 g of toluene, and washed with 20 g of a 1 N aqueous sodium hydroxide solution, and again washed twice with 100 g of water. The obtained toluene solution was purified by silica gel column chromatography, and then concentrated to obtain 68.2 g of a target compound as a viscous liquid.

The exemplary compound 3 represented by Chemical Formula [34] has a theoretical molecular weight of 1048.79, and a ratio of carbon atoms (theoretical value) of 75.58

### Synthesis Example 4

### Synthesis of the Exemplary Compound 4 Represented by Chemical Formula [5]

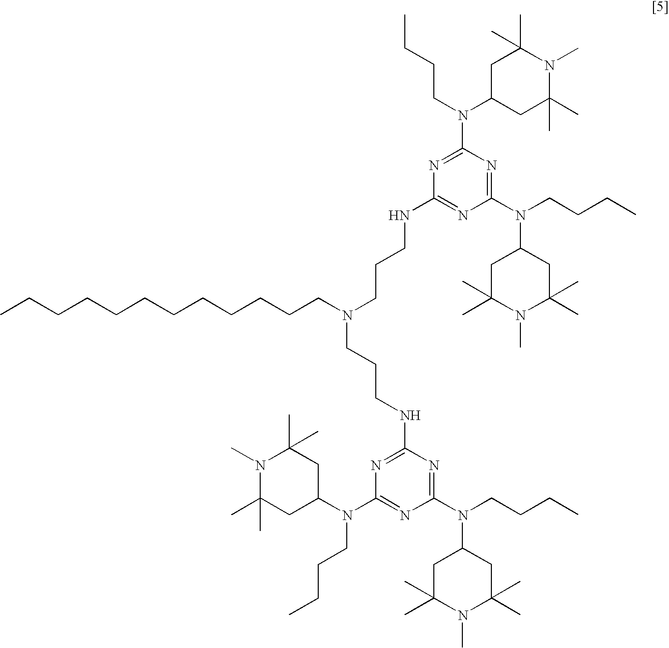

### (1) Synthesis of N,N-bis(2-cyanoethyl)dodecylamine

To a solution (150 ml) of 27.8 g of 1-aminododecane in ethanol was added dropwise 39.8 g of acrylonitrile over 0.5 hour at room temperature, and then 22.5 g of acetic acid was then added dropwise thereto over 0.5 hour. Then, the mixture was stirred at 77° C. for 10 hours. It was left to be cooled to room temperature, and 150 ml of water and 22.8 g of 28% aqueous ammonia were added thereto. The mixture was extracted with 330 ml of ethyl acetate. The organic layer obtained by liquid separation was washed with 100 ml of water and twice with 50 ml of saturated brine. It was dried over anhydrous magnesium sulfate, the solvent was removed by distillation, and the concentrated residue was purified by silica gel column chromatography to obtain 39.7 g of a target compound as a white solid.

¹H NMR (CDCl₃): δ=0.90(3H, t, J=6.5 Hz), 1.21-1.32 (18H, m), 1.32-1.51 (2H, m), 2.52-2.59 (6H, m), 2.82 (4H, t, 6.5 Hz)

### (2) Synthesis of N,N-bis(3-aminopropyl)dodecylamine

19.7 g of N,N-bis(2-cyanoethyl)dodecylamine, 1.97 g of RaneyCo, and 80 ml of 1,4-dioxane were charged into an autoclave, and subjected to a hydrogenation reaction at an initial hydrogen pressure of 8.2 MPa and 120° C. for 2 hours. The catalyst was removed by filtration, and the obtained filtrate was concentrated and dried to obtain 21.0 g of a target compound as a pale red oily substance. This procedure was carried out one more time to obtain a total of 40.8 g of a target compound as a pale red oily substance.

¹H NMR (CDCl₃): δ=0.88 (3H, t, J=6.5 Hz), 1.26-1.37 (18H, m), 1.37-1.47 (2H, m), 1.53-1.68 (4H, m), 2.35-2.47 (6H, m), 2.72-2.85 (4H, m)

GC-MS (m/z): 299

(3) 100 ml of a solution of 12.2 g of N,N-bis(3-aminopropyl)dodecylamine, 45.1 g of 2-chloro-4,6-bis(N-(1,2,2,6,6-pentamethyl piperidin-4-yl)butylamino)-1,3,5-triazine, and 11.1 g of potassium carbonate in N,N-dimethyl formamide (DMF) was stirred at 120° C. for 7 hours. It was left to be cooled to room temperature, and 350 ml of water was then added thereto. The mixture was extracted with 400 ml of ethyl acetate. The organic layer obtained by liquid separation was washed twice with 350 ml of water and once with 30 ml of saturated brine, and dried over anhydrous magnesium sulfate. The solvent was removed by distillation, and the concentrated residue was then purified by silica gel column chromatography to obtain 23.3 g of a target compound as a white solid.

¹H NMR (CDCl₃): δ=0.80-0.96 (15H, m), 1.09 (24H, s), 1.15 (24H, s), 1.10-1.70 (52H, m), 1.70 (4H, t, J=6.6 Hz), 2.24 (12H, s), 2.29-2.39 (2H, m), 2.45 (4H, t, J=6.6 Hz), 3.18-3.40 (8H, m), 3.38 (4H, dd, J=6.6, 12.5 Hz), 5.00-5.32 (4H, m)

MS (FD, m/z): 1354

Melting point: 67° C.

The exemplary compound 4 represented by Chemical Formula [5] has a theoretical molecular weight of 1355.2, and a ratio of carbon atoms (theoretical value) of 70.9

### Synthesis Example 5

### Synthesis of the Exemplary Compound 5 Represented by Chemical Formula [44] (N-dodecyl-2,2,6,6-tetramethyl piperidine-4-amine (TADA))

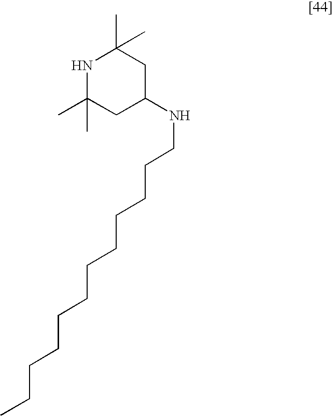

77.6 g (0.5 mol) of 2,2,6,6-tetramethyl-4-piperidone (TAA), 97.3 g (0.525 g) of laurylamine, and 1.8 g of 2% platinum carbon (50% water content) were charged into 116.4 g of methanol, and the mixture was allowed to undergo a reaction at a hydrogen pressure of 0.3 MPa and 50° C. for 2.5 hours. The catalyst was removed by filtration, desolvented, and then distilled to obtain 152.8 g of a target compound as a yellowish liquid.

The exemplary compound 5 represented by Chemical Formula [44] has a theoretical molecular weight of 324.59, and a ratio of carbon atoms (theoretical value) of 77.71

### Synthesis Example 6 (Synthesis Example)

### Synthesis of the Exemplary Compound 6 Represented by Chemical Formula [31] (N,N′,N″-tributyl-N,N′,N″-tris-(1,2,2,6,6-pentamethyl-4-piperidinyl)-[1,3,5]-triazine-2,4,6-triamine (T4M))

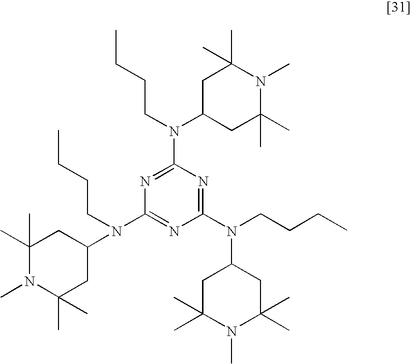

### (1) Synthesis of N-butyl-2,2,6,6-tetramethyl piperidine-4-amine (TABA)

108.7 g (0.7 mol) of 2,2,6,6-tetramethyl-4-piperidone (TAA), 53.8 g (0.735 mol) of butylamine, and 3.3 g of 2% platinum carbon (50% water content) were charged into 163.0 g of methanol, and the mixture was allowed to undergo a reaction at a hydrogen pressure of 0.3 MPa and 50° C. for 2.5 hours. This procedure was repeated four times. The catalyst was removed by filtration, desolvented, and then distilled to obtain 547.2 g (2.58 mol, yield 92%) of a target compound as a colorless transparent liquid.

### (2) Synthesis of N,N′,N″-tributyl-N,N′,N″-tris-(2,2,6,6-tetramethyl-4-piperidinyl)-[1,3,5]-triazine-2,4,6-triamine (TTABA)

326.5 g (1.54 mol) of TABA and 69.5 g (1.67 mol) of 96% sodium hydroxide were charged into 341 g of water, and heated to a temperature of 50° C., and 138.3 g (0.75 mol) of cyanuric chloride dissolved in 526 g of toluene was then added dropwise thereto over 3 hours. Then, the mixture was aged at 50° C. to 60° C. for 3 hours. The reaction mass was subjected to liquid separation while maintaining it at 60° C., and again washed with 294 g of warm water three times to obtain a solution of the reaction product in toluene. Thereafter, toluene was removed by distillation under reduced pressure at 80° C., and 483 g of dimethylacetamide (DMAc) and 54.4 g (0.39 mol) of potassium carbonate were charged thereinto. The mixture was heated to a temperature of 160° C., and 151.3 g (0.71 mol) TABA dissolved in 151.5 g of DMAc was then added dropwise thereto over 2 hours. Then, the mixture was aged for 18 hours under reflux. The reaction mass was cooled, and then discharged into 542 g of water added with 8.6 g (0.21 mol) of 96% sodium hydroxide. The reaction product was extracted with 526 g of toluene, and then washed with 515 g of water three times to obtain a solution of TTABA in toluene.

### (3) Synthesis of N,N′,N″-tributyl-N,N′,N″-tris-(1,2,2,6,6-pentamethyl-4-piperidinyl)-[1,3,5]-triazine-2,4,6-triamine (T4M)

101.9 g (3.39 mol) of paraformaldehyde was charged into the solution of TTABA in toluene obtained in (2), and heated to a temperature of 80° C., and 125.0 g (2.72 mol) of formic acid was then added dropwise thereto over 2 hours, and then the mixture was aged for 3 hours. The reaction mass was washed with 328 g of water added with 22.70 g (0.54 mol) of 96% sodium hydroxide while maintaining it at 80° C., and again washed twice with 324 g of warm water. The obtained toluene solution was diluted with addition of 896 g of toluene, purified by silica gel column chromatography, concentrated, and pulverized to obtain 457.7 g of a target compound as white powder (yield 81%/TCTA).

The exemplary compound 6 represented by Chemical Formula [31] has a theoretical molecular weight of 754.23, and a ratio of carbon atoms (theoretical value) of 71.66

### Synthesis Example 7 (Synthesis Example)

### Synthesis of the exemplary compound 7 represented by Chemical Formula [42] (N,N′,N″-trioctyl-N,N′,N″-tris-(1,2,2,6,6-pentamethyl-4-piperidinyl)-[1,3,5]-triazine-2,4,6-triamine (T8M))

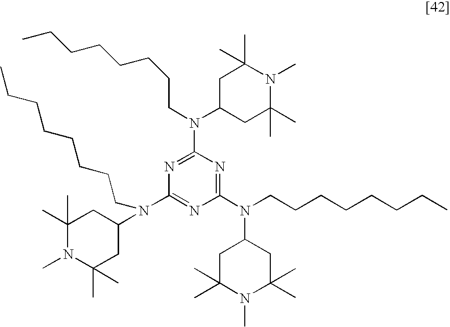

### (1) Synthesis of N-octyl-2,2,6,6-tetramethylpiperidine-4-amine (TAOA)

81.5 g (0.525 mol) of TAA, 64.7 g (0.5 mol) of octylamine, and 2.3 g of 2% platinum carbon (50% water content) were charged into 77.6 g of methanol, and the mixture was allowed to undergo a reaction at a hydrogen pressure of 0.3 MPa and 50° C. for 2.5 hours. This procedure was repeated four times. The catalyst was removed by filtration, desolvented, and then distilled to obtain 515.9 g of a target compound as a pale yellow transparent liquid (yield 96%).

### (2) Synthesis of N,N′,N″-trioctyl-N,N′,N″-tris (2,2,6,6-tetramethyl-4-piperidinyl)-[1,3,5]-triazine-2,4,6-triamine (TTAOA)

330.2 g (1.23 mol) of TAOA and 55.6 g (1.33 mol) of 96% sodium hydroxide were charged into 316 g of water, and heated to a temperature of 50° C., and 110.6 g (0.6 mol) of cyanuric chloride dissolved in 420 g of toluene was then added dropwise thereto over 3 hours. Then, the mixture was aged at 50° C. to 60° C. for 3 hours. The reaction mass was subjected to liquid separation while maintaining it at 60° C., and three times washed with 297 g of warm water to obtain a solution of the reaction product in toluene. Thereafter, toluene was removed by distillation under a reduced pressure at 80° C., and 467 g of DMAc and 43.5 g (0.31 mol) of potassium carbonate were charged thereinto, and the mixture was heated to a temperature of 160° C., and 153.0 g (0.57 mol) of TAOA dissolved in 153.0 g of DMAc was then added dropwise thereto over 1 hour. Then, the mixture was aged for 19 hours under reflux. The reaction mass was cooled, and then discharged into 519 g of water added with 7.1 g (0.17 mol) of 96% aqueous sodium hydroxide, and the reaction product was extracted with 498 g of toluene, and three times washed with 498 g of water to obtain a solution of TTAOA in toluene.

### (3) Synthesis of N,N′,N″-trioctyl-N,N′,N″-tris-(1,2,2,6,6-pentamethyl-4-piperidin-4-yl)-[1,3,5]-triazine-2,4,6-triamine (T8M)

81.0 g (2.70 mol) of paraformaldehyde was charged into the solution of TTAOA in toluene obtained in (2), and heated to a temperature of 80° C., and 99.5 g (2.16 mol) of formic acid was then added dropwise thereto over 2 hours. Then, the mixture was aged for 3 hours. The reaction mass was cooled, and then washed with 342 g of water added with 15.0 g (0.36 mol) of 96% sodium hydroxide, and washed twice with 341 g of warm water. The obtained toluene solution was diluted with addition of 713 g of toluene, purified by silica gel column chromatography, and concentrated to obtain 496.1 g of a target compound as a pale yellow transparent liquid. (yield 90%/TCTA)

The exemplary compound 7 represented by Chemical Formula [42] has a theoretical molecular weight of 922.55, and a ratio of carbon atoms (theoretical value) of 74.21

### Synthesis Example 8 (Synthesis Example)

### Synthesis of the Exemplary Compound 8 Represented by Chemical Formula [35] (N,N′,N″-tridodecyl-N,N′,N″-tris-(1,2,2,6,6-pentamethyl-4-piperidinyl)-[1,3,5]-triazine-2,4,6-triamine (T12M))

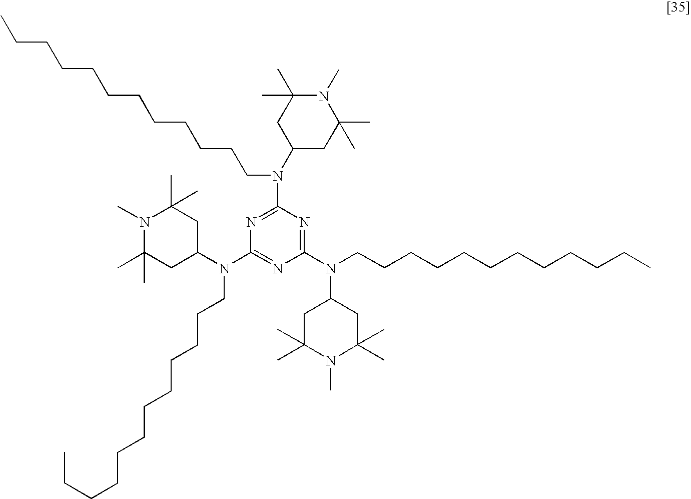

### (1) Synthesis of N-dodecyl-2,2,6,6-tetramethyl piperidine-4-amine (TADA)

77.6 g (0.5 mol) of TAA, 97.3 g (0.525 g) of dodecylamine, and 2.3 g of 2% platinum carbon (50% water content) were charged into 77.6 g of methanol, and subjected to reaction at a hydrogen pressure of 0.3 MPa and 50° C. over 2.5 hours. The catalyst was removed by filtration, desolvented, and then distilled to obtain 144.4 g of a target compound as a yellowish liquid (yield 89%).

### (2) Synthesis of N,N′,N″-tridodecyl-N,N′,N″-tris-(2,2,6,6-tetramethyl-4-piperidinyl)-[1,3,5]-triazine-2,4,6-triamine (TTADA)

194.8 g (0.6 mol) of TADA and 27.5 g (0.66 mol) of 96% sodium hydroxide were charged into 115 g of water, and heated to a temperature of 60° C., and 55.3 g (0.3 mol) of cyanuric chloride dissolved in 210 g of toluene was then added dropwise thereto over 1 hour. Then, the mixture was aged at 65° C. to 80° C. for 3 hours. The reaction mass was subjected to liquid separation while maintaining it at 80° C., and washed twice with 115 g of warm water to obtain a solution of reaction product in toluene. Thereafter, toluene was removed by distillation under reduced pressure at 80° C., and 274 g of DMAc and 21.8 g (0.16 mol) of potassium carbonate were charged thereinto. The mixture was heated to a temperature of 150° C., and 97.4 g (0.3 mol) of TADA dissolved in 97.4 g of DMAc was then added dropwise thereto over 2 hours. Then, the mixture was aged for 18 hours under reflux. The reaction mass was cooled, and then discharged into 290 g of water added with 3.8 g (0.09 mol) of 96% sodium hydroxide. The reaction product was extracted with 290 g of toluene and then twice with 290 g of water to obtain a solution of TTADA in toluene.

### (3) Synthesis of N,N′,N″-tridodecyl-N,N′,N″-tris-(1,2,2,6,6-pentamethyl-4-piperidin-4-yl)-[1,3,5]-triazine-2,4,6-triamine (T12M)

35.1 g (1.17 mol) of paraformaldehyde was charged into the solution of TTADA in toluene obtained in (2), and heated to a temperature of 80° C., and 49.7 g (1.08 mol) of formic acid was then added dropwise thereto over 1 hour, and then the mixture was aged for 3 hours. The reaction mass was cooled, and then washed with 125 g of water added with 7.9 g (0.19 mol) of 96% sodium hydroxide, and washed twice with 125 g of water. The obtained toluene solution was purified by silica gel column chromatography, and then concentrated to obtain 291.3 g of a target compound as a viscous solution. (yield 89%/TCTA)

The exemplary compound 8 represented by Chemical Formula [35] has a theoretical molecular weight of 1090.87, and a ratio of carbon atoms (theoretical value) of 75.97

### Synthesis Example 9

### Synthesis of N,N′-dibutyl-N″-dodecyl-N,N′-bis(2,2,6,6-tetramethyl-4-piperidyl)-1,3,5-triazine-2,4,6-triamine (Exemplary compound 9 represented by Chemical Formula [12])

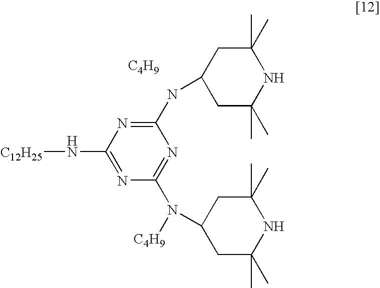

53.62 g of 2-chloro-4,6-bis(N-(2,2,6,6-tetramethyl-4-piperidyl)butylamino)-1,3,5-triazine and 8.29 g of potassium carbonate were charged into 100 g of dimethylacetamide. The mixture was heated to a temperature of 130° C., and 18.54 g of dodecylamine dissolved in 20 g of dimethylacetamide was then added dropwise thereto over 30 minutes. Then, the mixture was stirred at 130° C. to 145° C. for 2 hours. The reaction mixture was cooled, and then discharged into 200 g of water, and the reaction product was extracted with 150 g of hexane, and washed with 50 g of a 1 N aqueous sodium hydroxide solution, and again with 100 g of saturated brine (twice). The obtained hexane solution was dried over anhydrous magnesium sulfate, and purified by silica gel column chromatography to obtain 34.99 g of a target compound as a viscous liquid.

¹H NMR (CDCl₃): δ=0.65(2H, br), 0.80-0.98 (9H, m), 1.14 (12H, s), 1.00-1.90 (48H, m), 3.10-3.45 (6H, m), 4.97-5.40 (2H, m)

MS (FD, m/z): 685

### Synthesis Example 10

### Synthesis of N,N′,N″-tributyl-N,N′-bis(2,2,6,6-tetramethyl-4-piperidyl)-1,3,5-triazine-2,4,6-triamine (Exemplary Compound 10 Represented by Chemical Formula [38])

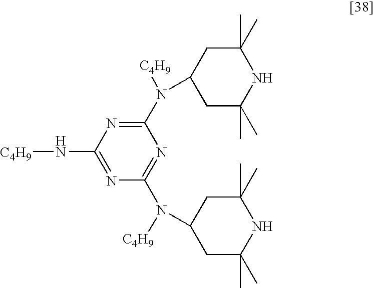

53.62 g of 2-chloro-4,6-bis(N-(2,2,6,6-tetramethyl-4-piperidyl)butylamino)-1,3,5-triazine and 8.29 g of potassium carbonate were charged into 100 g of dimethylacetamide. The mixture was heated to a temperature of 130° C., and 7.31 g of butylamine was then added dropwise thereto over 45 minutes. Then, the mixture was stirred at 130 to 145° C. for 3 hours. The reaction mixture was cooled, and then discharged into 200 g of water, and the reaction product was extracted with 150 g of hexane, and washed with 50 g of a 1 N aqueous sodium hydroxide solution, and again with 100 g of saturated brine (twice). The obtained hexane solution was dried over anhydrous magnesium sulfate, and purified by silica gel column chromatography to obtain 30.17 g of a target compound as a viscous liquid.

¹H NMR (CDCl₃): δ=0.65(2H, br), 0.80-0.98 (9H, m), 1.14 (12H, s), 1.00-1.90 (32H, m), 3.10-3.45 (6H, m), 4.97-5.40 (2H, m)

MS (FD, m/z): 572

### Synthesis Example 11

### Synthesis of N,N′,N″,N″-tetrabutyl-N,N′-bis(2,2,6,6-tetramethyl-4-piperidyl)-1,3,5-triazine-2,4,6-triamine (Exemplary Compound 11 Represented by Chemical Formula [40])

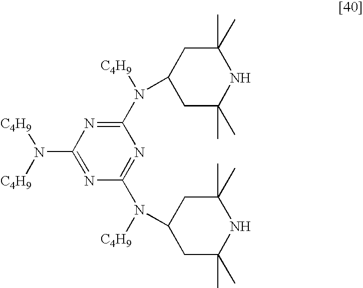

53.62 g of 2-chloro-4,6-bis(N-(2,2,6,6-tetramethyl-4-piperidyl)butylamino)-1,3,5-triazine and 8.29 g of potassium carbonate were charged into 60 g of dimethylacetamide. The mixture was heated to a temperature of 130° C., and 12.93 g of dibutylamine dissolved in 20 g of dimethylacetamide was then added dropwise thereto over 1 hour. Then, the mixture was stirred at 130 to 140° C. for 2 hours. The reaction mixture was cooled, and then discharged into 300 g of water, and the reaction product was extracted with 150 g of hexane, and washed with 20 g of a 1 N aqueous sodium hydroxide solution, and again with 100 g of water (three times). The obtained hexane solution was dried over anhydrous magnesium sulfate, and purified by silica gel column chromatography to obtain 58.96 g of a target compound as a viscous liquid.

¹H NMR (CDCl₃): δ=0.68(2H, br), 0.80-0.96 (12H, m), 1.09 (12H, s), 1.15 (12H, s), 1.10-1.70 (24H, m), 3.18-3.40 (4H, m), 3.46 (4H, t, J=7.2), 5.20-5.40 (2H, m)

MS (FD, m/z): 629

### Synthesis Example 12

### Synthesis of N,N′-dibutyl-N,N′-bis(2,2,6,6-tetramethyl-4-piperidyl)-N″, N″-dioctyl-1,3,5-triazine-2,4,6-triamine (Exemplary Compound 12 Represented by Chemical Formula [41])

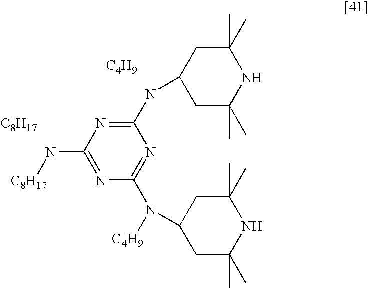

53.62 g of 2-chloro-4,6-bis(N-(2,2,6,6-tetramethyl-4-piperidyl)butylamino)-1,3,5-triazine and 8.29 g of potassium carbonate were charged into 60 g of dimethylacetamide. The mixture was heated to a temperature of 130° C., and 24.18 g of dioctylamine dissolved in 20 g of dimethylacetamide was then added dropwise thereto over 1 hour. Then, the mixture was stirred at 130 to 145° C. for 4 hours. The reaction mixture was cooled, and then discharged into 300 g of water, and the reaction product was extracted with 150 g of hexane, and washed with 20 g of a 1 N aqueous sodium hydroxide solution, and with 100 g of water (three times). The obtained hexane solution was dried over anhydrous magnesium sulfate, and purified by silica gel column chromatography to obtain 65.33 g of a target compound as a viscous liquid.

¹H NMR (CDCl₃): δ=0.69(2H, br), 0.80-0.98 (12H, m), 1.05 (12H, s), 1.15 (12H, s), 1.10-1.70 (40H, m), 3.20-3.40 (4H, m), 3.46 (4H, t, J=7.4), 5.18-5.37 (2H, m)

MS (FD, m/z): 741

### Synthesis Example 13

### Synthesis of N-dodecyl-N′,N″-bis(1,2,2,6,6-pentamethyl-4-piperidyl)-N′,N″-dioctyl-1,3,5-triazine-2,4,6-triamine (Exemplary compound 13 Represented by Chemical Formula [39])

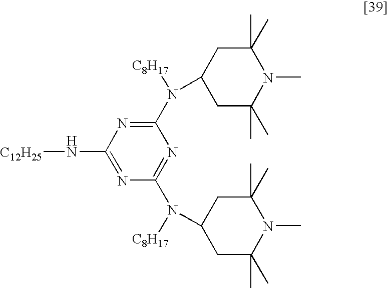

### (1) Synthesis of 2,2,6,6-tetramethyl-4-octylaminopiperidine

116.43 g of 2,2,6,6-tetramethyl-4-piperidone, 96.94 g of octylamine, and 2.01 g of platinum oxide were charged into 178.4 g of methanol, and subjected to a catalytic hydrogenation reaction under a normal pressure at 40° C. for 4 hours. The catalyst was removed by filtration, and then the solvent was removed by distillation to obtain 200.27 g of a target compound as a pale yellow liquid.

### (2) Synthesis of 2-chloro-4,6-bis(N-(1,2,2,6,6-pentamethyl-4-piperidyl)octylamino)-1,3,5-triazine

36.90 g of cyanuric chloride was charged into 866.9 g of toluene, and 107.39 g of 2,2,6,6-tetramethyl-4-octyl aminopiperidine was then added dropwise thereto at 20° C. to 25° C. over 1 hour. Thereafter, 290.0 g of a 20% aqueous potassium carbonate solution was then added dropwise thereto, and the mixture was stirred at 20° C. to 25° C. for 1 hour, and then at 80° C. to 85° C. for 10 hours. By the liquid separation operation, the organic layer was obtained, and this was washed with 500 g of water (three times). It was dried over anhydrous magnesium sulfate, and 19.16 g of 94% paraformaldehyde were then charged thereinto. The mixture was heated to a temperature of 85° C., and 19.73 g of formic acid was then added dropwise thereto over 1 hour. While maintaining the heating under reflux, a Dean-Stark device was used to remove water produced. The reaction mixture was cooled, and then washed with 500 g of a 1.1% aqueous potassium carbonate solution, and with 500 g of water (three times). The obtained toluene solution was dried over anhydrous magnesium sulfate, and the solvent was removed by distillation under reduced pressure to obtain 135.1 g of a target compound as a viscous liquid.

### (3) Synthesis of N-dodecyl-N′,N″-bis(1 2,2,6,6-pentamethyl-4-piperidyl)-N′,N″-dioctyl-1,3,5-triazine-2,4,6-triamine

50.0 g of 2-chloro-4,6-bis(N-(1 2,2,6,6-pentamethyl-4-piperidyl)octylamino)-1,3,5-triazine, 5.17 g of potassium carbonate, and 13.72 g of octylamine were charged into 80 g of dimethylacetamide, and the mixture was stirred at 130 to 145° C. for 3 hours. The reaction mixture was cooled, and then discharged into 300 g of water, and the reaction product was extracted with 150 g of hexane, and washed with 20 g of a 1 N aqueous sodium hydroxide solution, and with 100 g of water (three times). The obtained hexane solution was dried over anhydrous magnesium sulfate, and purified by silica gel column chromatography to obtain 55.88 g of a target compound as a viscous liquid.

¹H NMR (CDCl₃): δ=0.88(9H, t, J=6.8), 1.11 (12H, s), 1.16 (12H, s), 1.10-1.70 (52H, m), 2.26 (6H, s), 3.20-3.40 (6H, m), 4.60 (1H, t, J=5.5), 4.95-5.37 (2H, m)

MS (FD, m/z): 825

### Synthesis Example 14

### Synthesis of N-butyl-N′,N″-didodecyl-N′,N″-bis(2,2,6,6-tetramethyl-4-piperidyl)-1,3,5-triazine-2,4,6-triamine (the exemplary compound 14 represented by Chemical Formula [43])

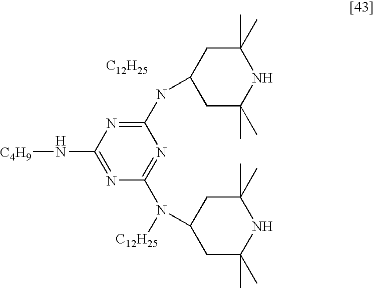

### (1) Synthesis of 2,2,6,6-tetramethyl-4-dodecylaminopiperidine

117.30 g of 2,2,6,6-tetramethyl-4-piperidone, 140.01 g of dodecylamine, and 1.74 g of platinum oxide were charged into 178.4 g of methanol, and subjected to a catalytic hydrogenation reaction under a normal pressure at 40° C. for 5 hours. The catalyst was removed by filtration, and the solvent was then removed by distillation to obtain 245.09 g of a target compound as a pale yellow liquid.

(2) Synthesis of 2-chloro-4,6-bis(N-(2,2,6,6-tetramethyl-4-piperidyl)dodecylamino)-1,3,5-triazine

36.90 g of cyanuric chloride was charged into 866.9 g of toluene, and 129.80 g of 2,2,6,6-tetramethyl-4-dodecyl aminopiperidine was then added dropwise thereto at 20° C. to 25° C. over 0.5 hour. Thereafter, 290.7 g of a 20% aqueous potassium carbonate solution was then added dropwise thereto, and the mixture was stirred at 20° C. to 25° C. for 1 hour, and then at 80° C. to 85° C. for 10 hours. The organic layer obtained by the liquid separation operation was washed with 500 g of water (twice). It was dried over anhydrous magnesium sulfate, and the solvent was removed by distillation under reduced pressure to obtain 140.30 g of a target compound as a pale yellow solid.

### (3) Synthesis of N-butyl-N′,N″-didodecyl-N′,N″-bis(2,2,6,6-tetramethyl-4-piperidyl)-1,3,5-triazine-2,4,6-triamine

53.25 g of 2-chloro-4,6-bis(N-(2,2,6,6-tetramethyl-4-piperidyl)dodecylamino)-1,3,5-triazine and 5.81 g of potassium carbonate were charged into 60 g of dimethylacetamide, the mixture was heated to a temperature of 130° C., and 5.45 g of butylamine dissolved in 20 g of dimethylacetamide was then added dropwise thereto over 1 hour. Thereafter, the mixture was stirred at 130 to 145° C. for 5 hours. The reaction mixture was discharged into 300 g of water, and the reaction product was extracted with 150 g of hexane, and washed with 20 g of a 1 N aqueous sodium hydroxide solution, and again with 100 g of water (three times). The obtained hexane solution was dried over anhydrous magnesium sulfate, and purified by silica gel column chromatography to obtain 53.42 g of a target compound as a viscous liquid.

¹H NMR (CDCl₃): δ=0.65(2H, br), 0.80-0.98 (9H, m), 1.14 (12H, s), 1.00-1.90 (64H, m), 3.10-3.45 (6H, m), 4.58 (1H, t, J=5.8), 4.97-5.40 (2H, m)

MS (FD, m/z): 797

Furthermore, the molecular weights of the hindered amine compounds obtained in Synthesis Examples 1 to 14 were measured, and the values of the weight average molecular weight in terms of polystyrene as measured by gel permeation chromatography (GPC), or a molecular weight as measured by mass analysis were substantially consistent to the values of the theoretical molecular weights. Further, the ratio of carbon atoms contained in the molecule structure was also measured, and the values of the carbon ratio as measured by means of a CHN elemental analyzer (CHNS-932 manufactured by LECO Corporation) were substantially consistent to the theoretical values.

### (Other Hindered Amine Compound)

A compound represented by Chemical Formula [45]: TINUVIN770 (trade name, manufactured by Chiba-Geigy Co., Ltd.)

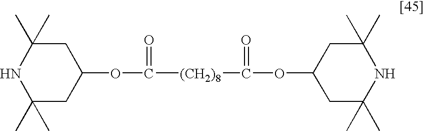

The theoretical molecular weight of TINUVIN770:480.72, and a ratio of carbon atoms (theoretical value) of 69.96

ADKSTAB LA-67 (trade name, manufactured by ADEKA Corporation), a condensate comprising the following compound:

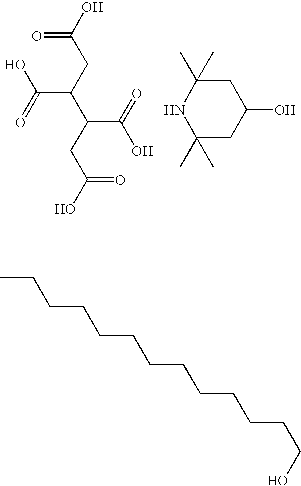

The molecular weight (measured value) of ADKSTAB LA-67:900, the ratio of carbon atoms (measured value): 72

CHIMASSORB 944 (trade name, manufactured by Chiba-Geigy Co., Ltd.)

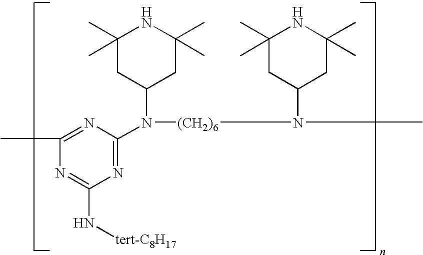

The molecular weight (measured value) of CHIMASSORB 944:2600, the ratio of carbon atoms (measured value): 70

CYASORB 3346 (trade name, manufactured by Cytec Industries Inc.)

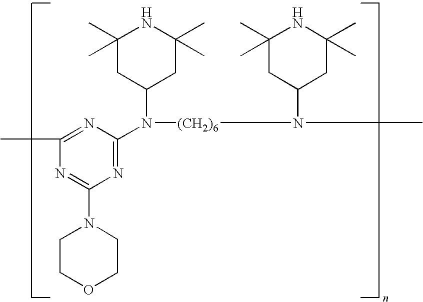

The molecular weight (measured value) of CYASORB 3346:1600, the ratio of carbon atoms (measured value): 66

Uvinul 5050H (trade name, manufactured by BASF)

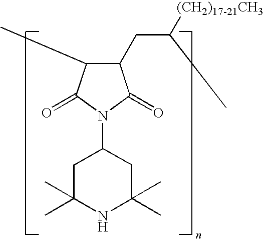

The molecular weight (measured value) of Uvinul 5050H: 3800, the ratio of carbon atoms (measured value): 77

### (Physical Properties of Hindered Amine Compound)

As for the hindered amine compound used in Examples, (1) hexane solubility, and (2) 5% by weight reducing temperature (heat resistance) were as shown in Table 1.

Furthermore, these physical properties were measured in the following manner.

(1) Hexane Solubility

The samples in an amount as shown in Table 1 were added into 100 g of n-hexane at 23° C., and the mixture was stirred for 1 hour, and thereafter the state was evaluated with naked eyes on the basis of following criteria.

### (Criteria for Evaluation)

S; Completely dissolved, C; Dissolved, but insoluble materials remained, and I.S.; Substantially not dissolved.

(2) 5% by Weight Reducing Temperature

The sample was heated at 5° C./minutes under nitrogen, and measured by means of TG8120 type TG-DTA device manufactured by Rigaku Corporation.

TABLE 1
Physical properties of the hindered amine compound

| | | | | | Heat resistance | |
|---|---|---|---|---|---|---|
| | | | | | 1% by weight | 5% by weight |
| | | | | | reducing | reducing |
| | Hexane solubility | | | | temperature | temperature |
| Names | 11 g | 25 g | 43 g | 100 g | (° C.) | (° C.) |
| Exemplary compound 1: | — | — | S | S | 271.6 | 333.4 |
| Chemical Formula [13] | | | | | | |
| Exemplary compound 2: | — | — | S | S | 292.9 | 366.7 |
| Chemical Formula [20] | | | | | | |
| Exemplary compound 3: | — | — | S | S | 246.2 | 377.1 |
| Chemical Formula [34] | | | | | | |
| Exemplary compound 4: | — | — | S | S | 292.7 | 357.2 |
| Chemical Formula [5] | | | | | | |
| Exemplary compound 5: | — | — | S | S | 103.3 | 188.1 |
| Chemical Formula [44] | | | | | | |
| Exemplary compound 6: | — | — | S | S | 250.7 | 320.2 |
| Chemical Formula [31] | | | | | | |
| Exemplary compound 7: | — | — | S | S | 291.5 | 340.1 |
| Chemical Formula [42] | | | | | | |
| Exemplary compound 8: | — | — | S | S | 330.1 | 378.2 |
| Chemical Formula [35] | | | | | | |
| Exemplary compound 9: | — | — | S | C | — | — |
| Chemical Formula [12] | | | | | | |
| Exemplary compound 10: | — | — | S | C | — | — |
| Chemical Formula [38] | | | | | | |
| Exemplary compound 11: | — | — | S | S | — | — |
| Chemical Formula [40] | | | | | | |
| Exemplary compound 12: | — | — | S | S | — | — |
| Chemical Formula [41] | | | | | | |
| Exemplary compound 13: | — | — | S | S | — | — |
| Chemical Formula [39] | | | | | | |
| Exemplary compound 14: | — | — | S | S | — | — |
| Chemical Formula [43] | | | | | | |
| TINUVIN 770: Chemical Formula | C | I.S. | — | — | 207.4 | 248.4 |
| [45] | | | | | | |
| ADKSTAB LA-67 | — | — | S | S | 215.0 | 288.8 |
| CHIMASSORB 944 | — | S | S | C | 226.2 | 378.0 |
| CYASORB 3346 | I.S. | — | — | — | 156.8 | 350.4 |
| Uvinul 5050H | — | — | S | S | 238.3 | 325.1 |

### (Phosphorus Stabilizer)

As the phosphorus stabilizer, the following compounds were used.
Sumilizer GP (trade name, manufactured by Sumitomo Chemical Co., Ltd.)

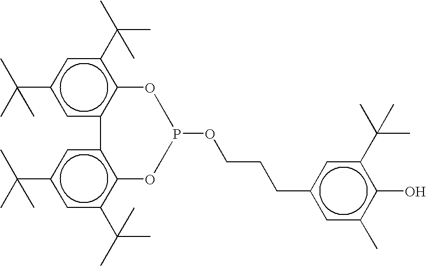

ADKSTAB HP-10 (trade name, manufactured by ADEKA Corporation)

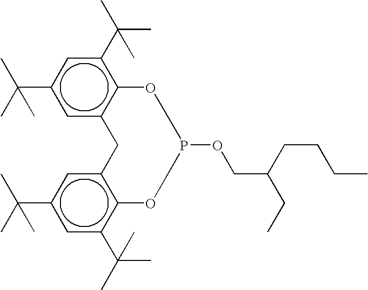

(Hydrophilic Stabilizer)

As the hydrophilic stabilizer, the following compound was used.
Pentaaerythritol monostearyl ester (trade name: Exepal PE-MS, manufactured by Kao Corporation)

As the UV absorbent, the following compound was used.
Tinuvin 328 (trade name, manufactured by Chiba-Geigy Co., Ltd.)

### [Process for Preparing Resin Composition A]

### (Preparation of Catalyst)

VO(OC₂H₅) Cl₂ was diluted with cyclohexane to prepare a vanadium catalyst in which the vanadium concentration is 6.7 mmol/L-cyclohexane. Ethylaluminum sesquichloride (Al(C₂H₅)_{1.5}Cl_{1.5}) was diluted with cyclohexane to prepare an organoaluminum compound catalyst in which the aluminum concentration is 107 mmol/L-hexane.

### (Polymerization)

The copolymerization reaction of ethylene and tetracyclo [4.4.0.1^{2,5},1^{7,10}]-3-dodecene was continuously carried out by using a polymerization apparatus with stirrer (inner diameter of 500 mm, reaction volume of 100 L).

Upon carrying out the copolymerization reaction, the vanadium catalyst prepared by the above process was supplied to the polymerization apparatus so that the vanadium catalyst concentration became 0.6 mmol/L, related to cyclohexane in the polymerization apparatus used as the polymerization solvent.

In addition, ethylaluminum sesquichloride which is organoaluminum compound was supplied to the polymerization apparatus so as to be Al/V=8.0. The copolymerization reaction was continuously carried out at the polymerization temperature of 11° C. and the polymerization pressure of 1.8 kg/cm²G.

### (Demineralization)

The polymerization reaction was terminated by adding water and NaOH solution having the concentration of 25% by weight as a pH adjuster, to ethylene-tetracyclo [4.4.0.1^{2,5}, 1^{7,10}]-3-dodecene copolymer solution extracted from the polymerization apparatus, and the catalyst residue existing in the copolymer was removed from the copolymer solution. (demineralization)

To the cyclohexane solution of ethylene tetracyclo [4.4.0.1^{2,5},1^{7,10}]-3-dodecene copolymer subjected to the demineratization treatment, the liquid stabilizer was added in the amount described in the Table 2, and then the cyclohexane solution of the copolymer having the concentration of the copolymer in the cyclohexane solution is 5% by weight was supplied to the double-pipe heater (outer pipe diameter of 2B, inner pipe diameter of 3/4B, and length of 21 m) using steam of 20 kg/cm²G as heat source, in the amount of 150 kg/H to be heated to 180° C., in the heating process.

Next, by the use of the double-pipe flash dryer (outer pipe diameter of 2B, inner pipe diameter of 3/4B, and length of 27 m) using steam of 25 kg/cm²G as heat source, and a flash hopper (volume of 200 L), from the cyclohexane solution of the copolymer subjected to the heating process, cyclohexane which is a polymerization solvent as well as most of the unreacted monomers were removed, to obtain a flash dried cyclic olefin random copolymer in the melt state. Thereafter, by the use of the twin screw kneading extruder with a vent, the cyclic olefin random copolymer in the melt state was charged to the resin insertion site of the extruder, and then the melting stabilizer were added in the amount described in the Table 2 to a cylinder site which is located in a downstream side as compared with a vent site, while being aspirated through the trap by the vacuum pump for the purpose of removing a volatile component from the vent site, and kneaded and mixed in the downstream side as compared with a vent site of the extruder. Subsequently, the resulting product was made into a pellet by an under water pelletizer equipped to outlet of the extruder, and the obtained pellet was dried with heated air at 100° C. for 4 hours.

Further, the addition amount of each of the hindered amine compound, the phosphorus stabilizer, and the hydrophilic stabilizer as shown in the Table is an addition amount (parts by mass), based on 100 parts by mass of the polymer. Further, as the melting stabilizer, the stabilizer as described in Table 2 was put into a vessel, and heat-molten at a predetermined temperature for 10 hours was used. This is also applied to the processes for preparation of the resin compositions A(H), B, and C.

### [Process for Preparing Resin Composition A(H)]

The same procedure as the process for preparing the resin composition A was carried out, except that a solution of the ethylene/tetracyclo[4.4.0.1^{2,5}.1^{7,10}]-3-dodecene copolymer in cyclohexane solution (polymer concentration: 7.7% by weight) obtained by the process for preparing the resin composition A was continuously hydrogenated by using a nickel/diatomaceous earth catalyst (N112 manufactured by Nikki Chemical Co., Ltd.) under the condition of a reaction temperature of 100° C., a reaction pressure of 1 MPa, and LHSV=5/hr to hydrogenate the copolymer, thereby preparing the resin composition A(H).

[Process for Preparing Resin composition B]

To a pressure-resistant container purged with nitrogen, 7.68 kg of styrene and 0.32 kg of isoprene were added, mixed, stirred, and 32 kg of anhydrous cyclohexane, 0.4 kg of mixed monomer, and 0.01 kg of dibutyl ether were charged, 0.0454 kg of hexane solution (concentration of 15%) of n-butyllithium was added while stirring at 50° C. to carried out the polymerization. After a lapse of 0.5 hour from starting the polymerization, 7.6 kg of a mixed monomer was added to the solution continuously over 1 hour. After a lapse of 0.5 hour from completing the addition of the mixed monomer, 0.01 kg of isopropyl alcohol was added to the solution, to obtain a polymerization reaction solution in which styrene-isoprene random copolymer was dissolved.

Next, to 40 kg of the polymerization reaction solution was added 0.3 kg of a stabilized nickel hydrogenation catalyst E22U (60% nickel supported silica-alumina carrier manufactured by Nikki Chemical Co., Ltd.), and mixed to obtain a mixed solution, and the mixed solution was charged to an autoclave. To the autoclave, a hydrogen gas was supplied, and the hydrogenation reaction was carried out in the autoclave at 160° C. and 4.5 MPa for 6 hours, wile being stirred. After the hydrogenation reaction is completed, the hydrogenation catalyst is removed by filtering to obtain a colorless transparent solution.

To the cyclohexane solution of hydrogenated styrene-isoprene random copolymer, above liquid stabilizer was added in the amount described in the Table 2, and then the cyclohexane solution of the copolymer having the concentration of the copolymer in the cyclohexane solution is 5% by weight was supplied to the double-pipe heater (outer pipe diameter of 2B, inner pipe diameter of 3/4B, and length of 21 m) using steam of 20 kg/cm²G as heat source in the amount of 150 kg/H to be heated to 180° C., in the heating process.

Next, by the use of the double-pipe flash dryer (outer pipe diameter of 2B, inner pipe diameter of 3/4B, and length of 27 m) using steam of 25 kg/cm² G as heat source, and a flash hopper (volume of 200 L), from the cyclohexane solution of the copolymer subjected to the heating process, cyclohexane which is a polymerization solvent as well as most of the unreacted monomers were removed, to obtain a flash dried vinyl alicyclic hydrocarbon polymer in the melt state. Thereafter, by the use of the twin screw kneading extruder with a vent, the vinyl alicyclic hydrocarbon polymer in the melt state was charged to the resin insertion site of the extruder, and then the melting stabilizer was added in the amount described in the Table 2 to a cylinder site which is located in a downstream side as compared with a vent site, while being aspirated through the trap by the vacuum pump for the purpose of removing a volatile from the vent site, and kneaded and mixed in the downstream side as compared with a vent site of the extruder. Subsequently, the resulting product was made into a pellet by an under water pelletizer equipped to the outlet of the extruder, and the obtained pellet was dried with heated air at 100° C. for 4 hours.

### [Process for Preparing Resin Composition C]

To 50 kg of dehydrated cyclohexane, 0.082 kg of 1-hexene, 0.015 kg of dibutyl ether, and 0.03 kg of triisobutylaluminum were charged to the reactor and mixed at room temperature under the nitrogen atmosphere, and then 20 kg of 8-methyl-tetracyclo [4.4.0.1^{2,5}.1^{7,10}]-dodeca-3-ene (methyltetracyclododecene, hereinafter abbreviated to as “MTD”) and 8 kg of tungsten hexachloride (0.7% toluene solution) were added thereto continuously over 2 hours to carry out the polymerization while maintaining the temperature of 45° C.

To the polymerization solution, 0.106 kg of butyl glycidyl ether and 0.052 kg of isopropyl alcohol were added to inactivate the polymerization catalyst and to terminate the polymerization reaction. Thereafter, to 70 kg of the reaction solution containing the obtained ring-opening polymer, 30 kg of cyclohexane was added, and 0.5 kg of a nickel-alumina catalyst (manufactured by Nikki Chemical Co., Ltd.) was further added as the hydrogenation catalyst. To the solution, hydrogen was supplied to pressurize at 5 MPa, and the solution was heated to 200° C. while stirring to react for 4 hours. Thereafter, the hydrogenation catalyst was removed by the filtration to obtain a colorless transparent solution.

To the cyclohexane solution of a hydrogenated MTD ring-opening polymer was added the liquid stabilizer in the amount described in the Table 2, and then the cyclohexane solution of the copolymer having the concentration of the copolymer in the cyclohexane solution is 5% by weight was supplied to the double-pipe heater (outer pipe diameter of 2B, inner pipe diameter of 3/4B, and length of 21 m) using steam of 20 kg/cm²G as heat source, in the amount of 150 kg/H to be heated to 180° C., in the heating process.

Next, by the use of the double-pipe flash dryer (outer pipe diameter of 2B, inner pipe diameter of 3/4B, and length of 27 m) using steam of 25 kg/cm²G as heat source, and a flash hopper (volume of 200 L), from the cyclohexane solution of the copolymer subjected to the heating process, cyclohexane which is a polymerization solvent as well as most of the unreacted monomers were removed, to obtain a flash dried hydrogenation product of the MTD ring-opening polymer in the melt state. Thereafter, by the use of the twin screw kneading extruder with a vent, the hydrogenation product of the MTD ring-opening polymer in the melt state was charged from the resin injection site of the extruder, and then the melting stabilizer was added in the amount described in the Table 2 to a cylinder site which is located in a downstream side as compared with a vent site, while being aspirated through the trap by the vacuum pump for the purpose of removing a volatile from the vent site, and kneaded and mixed in the downstream side as compared with a vent site of the extruder. Subsequently, the resulting product was made into a pellet by an under water pelletizer equipped to outlet of the extruder, and the obtained pellet was dried with heated air at 100° C. for 4 hours.

### Examples 1 to 23 and Comparative Examples 1 to 6

According to the above preparation process, the resin compositions A, A (H), B, and C containing the hindered amine compound, the phosphorus stabilizer, and the hydrophilic stabilizer as shown in the Table 2 were prepared. The characteristics of the molded product were evaluated by carrying out the following tests.

### (Haze and Spectral Light Transmittance)

The resin composition was subjected to an injection molding by the injection molding machine (IS-50 manufactured by TOSHIBA MACHINE CO., LTD) which was set to a cylinder temperature of 260° C. and a mold temperature of 125° C., to prepare a test piece having an optical surface of 45 mmφ×3 mm (thickness), and a haze and a spectral light transmittance (405 nm and 650 nm) were measured. The results are shown in the Table 2.

(Reliability Evaluation with Blue-Violet Laser Beam)

By using the above test piece, the blue-violet laser light of 405±10 nm and 25 mW/mm² was radiated to the center of the test piece positioned in a constant-temperature bath of 70° C. for 336 hours by using a laser diode (TC4030S-F405ASU manufactured by Neo Arc Co., Ltd.). Before the irradiation, after 168 hours of the irradiation, and after completion of the irradiation, wavefront RMS values of the 1 mmφ in the center of the test piece were measured, and temporal changes were evaluated. For the RMS value measurement, a laser interferometer (PTI 250RS manufacture by ZYGO Corporation (linearly-polarization specifications) was used. Further, the irradiation portion in the test piece was observed by a stereomicroscope, and white turbidity and adhesion of foreign matters were confirmed. The results were represented by following symbols. The results are shown in the Table 2.

### (Evaluation of RMS Values)

◯: No change of RMS value

Δ: Rate of change of RMS value was observed less than 0.01λ.

x: RMS value was changed by 0.01 λ or more. Alternatively, the measurement was impossible.

### (Evaluation of White Turbidity and Adhesion of Foreign Matters)

∇: White turbidity and adhesion of foreign matters were remarkably observed.

(Test on Weather Resistance)

The above test piece was subjected to an exposure test at a BP temperature of 63° C., 18 minutes for water spray in every 120 minutes, a spray pressure of 1.0 kgf/cm², and a spray amount of 2100 cc/min, using a sunshine carbon arc light system weatherometer (WEL-SUN-HC-E) manufactured by Suga Testing Machinery. The hazes were measured before the test, after 1000 hours, after 2000 hours, and after 4000 hours. The results are shown in the Table 2.

TABLE 2

| | | | | Melting stabilizer | | | | | | Laser reliability | | | Weather | | | |
|---|---|---|---|---|---|---|---|---|---|---|---|---|---|---|---|---|
| | | | | | | Addi- | Spectral light | | | evaluation | | | resistance test | | | |
| | | Solution stabilizer | | | Melt- | tion | transmittance/ | | | Results of | | | (sunshine | | | |
| | Resin | | Addition | | ing | amount | haze | | | evaluation of | | | weatherometer) | | | |
| | com- | | amount | | con- | (parts | | 405 | 650 | RMS/appearance | | | Haze (%) | | | |
| | po- | | (parts by | | di- | by | haze | nm | nm | | 168 | 336 | | 1000 | 2000 | 4000 |
| | sition | Types | mass) | Types | tion | mass) | (%) | (%) | (%) | 0 hr | hr | hr | 0 h | h | h | h |
| Example 1 | A | SUMILIZER | 0.04 | Exemplary | *1 | 0.2 | 0.2 | 87.8 | 91.1 | ◯ | ◯∇ | Δ∇ | | | | |
| | | GP | | Compound 1 | | | | | | | | | | | | |
| Example 2 | A | SUMILIZER | 0.30 | Exemplary | *1 | 1.0 | 0.0 | 87.8 | 91.2 | ◯ | ◯ | ◯∇ | | | | |
| | | GP | | Compound 2 | | | | | | | | | | | | |
| Example 3 | A | ADKSTAB | 0.30 | Exemplary | *1 | 1.0 | 0.1 | 87.7 | 91.1 | ◯ | ◯ | ◯∇ | | | | |
| | | HP-10 | | Compound 2 | | | | | | | | | | | | |
| Example 4 | A(H) | SUMILIZER | 0.60 | Exemplary | *1 | 4.0 | 0.2 | 89.8 | 91.1 | ◯ | ◯ | ◯∇ | | | | |
| | | GP | | Compound 3 | | | | | | | | | | | | |
| Example 5 | A | SUMILIZER | 0.20 | Exemplary | *1 | 1.0 | 0.1 | 88.0 | 91.0 | ◯ | ◯ | ◯∇ | 0.1 | 0.1 | 0.1 | 1.5 |
| | | GP | | Compound 3 | | | | | | | | | | | | |
| Example 6 | A | SUMILIZER | 0.20 | None | | None | 0.1 | 87.7 | 91.1 | ◯ | ◯ | ◯∇ | | | | |
| | | GP | | | | | | | | | | | | | | |
| | | Exemplary | 1.00 | | | | | | | | | | | | | |
| | | Compound 3 | | | | | | | | | | | | | | |
| Example 7 | A(H) | SUMILIZER | 0.20 | Exemplary | *1 | 1.0 | 0.0 | 90.3 | 91.2 | ◯ | ◯ | ◯∇ | | | | |
| | | GP | | Compound 3 | | | | | | | | | | | | |
| | | | | hydrophilic | *1 | 1.8 | | | | | | | | | | |
| | | | | stabilizer | | | | | | | | | | | | |
| Example 8 | A | SUMILIZER | 0.20 | Exemplary | *2 | 2.0 | 0.2 | 87.8 | 91.2 | ◯ | ◯ | ◯∇ | | | | |
| | | GP | | Compound 3 | | | | | | | | | | | | |
| Example 9 | A | SUMILIZER | 0.20 | Exemplary | *3 | 2.0 | 0.2 | 87.5 | 91.2 | ◯ | ◯ | ◯∇ | | | | |
| | | GP | | Compound 3 | | | | | | | | | | | | |
| Example 10 | A | SUMILIZER | 0.20 | Exemplary | *2 | 2.0 | 0.3 | 86.7 | 91.2 | ◯ | ◯ | ◯∇ | | | | |
| | | GP | | Compound 4 | | | | | | | | | | | | |
| Example 11 | A | SUMILIZER | 0.20 | Exemplary | *3 | 2.0 | 0.2 | 85.4 | 91.0 | ◯ | ◯ | ◯∇ | | | | |
| | | GP | | Compound 4 | | | | | | | | | | | | |
| Example 12 | A | SUMILIZER | 0.25 | ADKSTAB | *1 | 1.0 | 0.4 | 86.8 | 90.7 | ◯ | ◯∇ | ◯∇ | | | | |
| | | GP | | LA-67 | | | | | | | | | | | | |
| | | | | hydrophilic | *1 | 2.3 | | | | | | | | | | |
| | | | | stabilizer | | | | | | | | | | | | |
| Example 13 | A | SUMILIZER | 0.10 | CHIMASS | *3 | 2.5 | 0.5 | 87.6 | 90.5 | ◯ | ◯∇ | Δ∇ | | | | |
| | | GP | | ORB944 | | | | | | | | | | | | |
| Comparative | A | SUMILIZER | 0.10 | CYASORB | *3 | 2.5 | 0.7 | 87.0 | 90.2 | ◯∇ | ◯∇ | X∇ | | | | |
| Example 1 | | GP | | 3346 | | | | | | | | | | | | |
| Example 14 | B | SUMILIZER | 0.10 | Exemplary | *2 | 0.6 | 0.0 | 91.0 | 92.1 | ◯ | ◯ | ◯∇ | | | | |
| | | GP | | Compound 2 | | | | | | | | | | | | |
| Example 15 | C | SUMILIZER | 0.30 | Exemplary | *2 | 1.5 | 0.3 | 89.8 | 91.3 | ◯ | ◯∇ | Δ∇ | | | | |
| | | GP | | Compound 3 | | | | | | | | | | | | |
| Example 16 | A | None | | Exemplary | *1 | 1.5 | 0.1 | 88.0 | 91.1 | ◯ | ◯∇ | Δ∇ | | | | |
| | | | | Compound 1 | | | | | | | | | | | | |
| Example 17 | A | SUMILIZER | 0.40 | Exemplary | *2 | 3.0 | 0.4 | 86.6 | 91.0 | ◯ | ◯ | ◯∇ | | | | |
| | | GP | | Compound 8 | | | | | | | | | | | | |
| Example 18 | A(H) | SUMILIZER | 0.20 | Exemplary | *2 | 0.7 | 0.1 | 88.8 | 91.2 | ◯ | ◯ | ◯∇ | | | | |
| | | GP | | Compound 6 | | | | | | | | | | | | |
| Example 19 | A(H) | SUMILIZER | 0.20 | Exemplary | *2 | 0.7 | 0.1 | 89.2 | 91.1 | ◯ | ◯ | ◯∇ | | | | |
| | | GP | | Compound 7 | | | | | | | | | | | | |
| Example 20 | A(H) | SUMILIZER | 0.20 | Exemplary | *2 | 0.7 | 0.1 | 89.3 | 91.2 | ◯ | ◯ | ◯ | 0.1 | 0.1 | 0.1 | 0.3 |
| | | GP | | Compound 8 | | | | | | | | | | | | |
| Example 21 | A(H) | SUMILIZER | 0.10 | Exemplary | *2 | 0.15 | 0.0 | 89.4 | 91.2 | ◯ | ◯ | ◯∇ | | | | |
| | | GP | | Compound 8 | | | | | | | | | | | | |
| Example 22 | B | SUMILIZER | 0.15 | Exemplary | *2 | 0.5 | 0.0 | 90.3 | 91.4 | ◯ | ◯∇ | ◯∇ | | | | |
| | | GP | | Compound 8 | | | | | | | | | | | | |
| Example 23 | C | SUMILIZER | 0.30 | Exemplary | *2 | 1.0 | 0.1 | 89.7 | 91.5 | ◯ | ◯ | ◯∇ | | | | |
| | | GP | | Compound 8 | | | | | | | | | | | | |
| Comparative | A | SUMILIZER | 0.30 | Exemplary | *1 | 0.03 | 0.1 | 87.3 | 91.0 | Δ∇ | X∇ | X∇ | 0.1 | 0.3 | 1.9 | 6.3 |
| Example 2 | | GP | | Compound 1 | | | | | | | | | | | | |

| Comparative | A | SUMILIZER | 0.30 | Exemplary | *1 | 7.0 | 4.2 | 79.2 | 86.5 | Deformed during | | |
|---|---|---|---|---|---|---|---|---|---|---|---|---|
| Example 3 | | GP | | Compound 1 | | | | | | the test | | |
| Comparative | A | SUMILIZER | 0.30 | Exemplary | *1 | 1.0 | 1.5 | 70.3 | 89.4 | Δ∇ | X∇ | X∇ |
| Example 4 | | GP | | Compound 5 | | | | | | | | |
| Comparative | A | SUMILIZER | 0.30 | TINUVIN770 | *2 | 1.0 | 0.8 | 86.5 | 90.6 | Δ∇ | X∇ | X∇ |
| Example 5 | | GP | | | | | | | | | | |

| | | | | | | | | | | |
|---|---|---|---|---|---|---|---|---|---|---|
| Comparative | A | SUMILIZER | 0.30 | Uvinul | *3 | 1.0 | 3.8 | 74.9 | 87.7 | Deformed during |
| Example 6 | | GP | | 5050H | | | | | | the test |

Melting condition
*1: 80° C., 10 hrs,
*2: 130° C., 10 hrs,
*3: 180° C., 10 hrs

Further, in Example 1, the resin composition A in which the content of an iron atom was 0.4 ppm or 5.6 ppm was prepared, and it was confirmed that the molded product obtained from the resin composition in which the content of an iron atom was 0.4 ppm had temporal deterioration of the reliability evaluation results in the blue-violet laser light, as compared to the molded product obtained from the resin composition in which the content of an iron atom was 5.6 ppm.

### Example 24

The copolymerization reaction of ethylene and 1,4-methano-1,4,4a,9a-tetrahydrofluorene (MTHF) having the following structure was carried out as below.

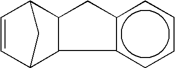

Into a glass-made reaction vessel having a 500 ml volume that had been equipped with a stirring device was flowed nitrogen as an inert gas at a flow rate of 25 Nl/hr for 30 minutes, and 250 ml of cyclohexane and 10 ml of MTHF as a cyclic olefin, and 0.56 ml of a solution of ethyl aluminum sesquichloride ((C₂H₅)_{1.5}AlCl_{1.5}) in decane (concentration: 2.214 mM/ml) were introduced thereinto, and the polymerization solvent was stirred at 500 to 600 rpm while adjusting the solvent temperature to 25° C. The solvent temperature reached 25° C., and in addition to nitrogen, ethylene and hydrogen were flowed into the reaction vessel at feed rates of 25 Nl/hr and 2 Nl/hr, respectively, and after 10 minutes, 0.46 ml of a solution of VO(OC₂H₅)Cl₂ in hexane (concentration: 0.271 mM/ml) and 5 ml of hexane, which had been place in the dropping funnel on the top of the reaction vessel in advance were introduced to the solution to initiate the polymerization.

After 5 minutes, 5 ml of methanol was added to stop the polymerization to obtain a polymerization solution containing an ethylene/cyclic olefin (MTHF) copolymer. Thereafter, the polymerization solution was transferred to a separately prepared beaker having a 1 L volume, and additionally, 5 ml of concentrated hydrochloric acid and a stirrer were added thereto to stir for 2 hours under strongly stirring and to carry out a demineralization operation. To a beaker to which acetone at a 3-fold volume based on the polymerization solution was added, the polymerization solution after demineralization was added while stirring to precipitate, and the precipitated copolymer was separated from the filtrate by filtration. The obtained polymer containing the solvent was dried under reduced pressure at 130° C. for 12 hours to obtain 2.4 g of an ethylene/MTHF copolymer.

The content of the cyclic olefin in the obtained ethylene/MTHF copolymer as calculated from a ¹³C-NMR spectrum was 31.1 mol %, and the glass transition temperature was 125° C. This ethylene/MTHF copolymer was pulverized by a freezing pulverizer, and then 30 mg of the exemplary compound 2 and 10 mg of Sumilizer GP were mixed with 2.1 g of the ethylene/MTHF copolymer, and a press machine at 240° C. was used to obtain a press sheet having a thickness of 100 microns. The obtained test piece had good transparency. This press sheet was left outdoor for 1 year, but no change could be found.

### Example 25

The polymerization was carried out in the same manner as in Example 17, except that 15 g of cyclopentadienebenzaine adduct (BNBD) represented by the following Formula was used instead of MPBH of Example 1, to obtain 1.7 g of an ethylene/BNBD copolymer.

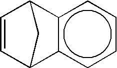

The content of the cyclic olefin in the obtained ethylene/BNBD copolymer as calculated from a ¹³C-NMR spectrum was 37.8 mol %, and the glass transition temperature was 133° C. This ethylene/MTHF copolymer was pulverized by a freezing pulverizer, and then 20 mg of the exemplary compound 3 and 7 mg of Sumilizer GP were mixed with 1.4 g of the ethylene/MTHF copolymer, and a press machine at 240° C. was used to obtain a press sheet having a thickness of 100 microns. The obtained test piece had good transparency. This press sheet was left outdoor for 1 year, but no change could be found.

### Examples 26 to 31, and Comparative Example 7

### Preparation of Raw Material Resin Composition

By the same preparation process as in Examples 1 to 16, the raw material resin composition comprising the phosphorus stabilizer/the hydrophilic stabilizer as shown in Table 3 was prepared.

TABLE 3

| | | Liquid stabilizer | | Melting stabilizer | | |
|---|---|---|---|---|---|---|
| | | | Addition | | | Addition |
| | | | amount | | | amount |
| | Resin | | (parts by | | Melting | (parts by |
| | Composition | Type | mass) | Type | condition | mass) |
| Raw | A(H) | Sumilizer | 0.15 | Hydrophilic | 80° C., 10 Hr | 1.5 |
| material | | GP | | stabilizer | | |

This raw material was fed to a JSW TEX44 biaxial extruder, and HALS and the UV absorbent as described in Table 4 were fed from a vent, and mixed at a resin temperature of 265° C. to obtain a pellet.

TABLE 4

| | HALS | | UV absorbent | |
|---|---|---|---|---|
| | | Addition | | Addition |
| | | amount (parts | | amount (parts |
| | Type | by mass) | Type | by mass) |
| Example 26 | Exemplary | 0.7 | Tinuvin | 0.5 |
| | Compound 9 | | 328 | |
| Example 27 | Exemplary | 0.7 | Tinuvin | 0.5 |
| | Compound 10 | | 328 | |
| Example 28 | Exemplary | 0.7 | Tinuvin | 0.5 |
| | Compound 11 | | 328 | |
| Example 29 | Exemplary | 0.7 | Tinuvin | 0.5 |
| | Compound 12 | | 328 | |
| Example 30 | Exemplary | 0.7 | Tinuvin | 0.5 |
| | Compound 13 | | 328 | |
| Example 31 | Exemplary | 0.7 | Tinuvin | 0.5 |
| | Compound 14 | | 328 | |
| Comparative | None | — | Tinuvin | 0.5 |
| Example 7 | | | 328 | |

These pellets were subjected to an injection molding by the injection molding machine (IS-50 manufactured by TOSHIBA MACHINE CO., LTD.) which was set to a cylinder temperature of 260° C. and a mold temperature of 125° C., to prepare a square plate of 65 mm×35 mm×2 mm (thickness), and a haze was measured. The results are shown in the Table 5.

After this square plate was left outdoor for exposure to a direct sunlight and rainfall for 1 and 3 years, haze and appearance tests were carried out, and the results thereof are shown in Table 5.

TABLE 5

| | | After 1 years | | After 3 years | |
|---|---|---|---|---|---|
| | Haze | Haze | Examination of | | Examination of |
| | (%) | (%) | appearance | Haze (%) | appearance |
| Example 26 | 0.2 | 0.2 | Not changed | 3.5 | White turbid |
| Example 27 | 0.3 | 0.2 | Not changed | 2.0 | White turbid |
| Example 28 | 0.2 | 0.3 | Not changed | 1.1 | White turbid |
| Example 29 | 0.3 | 0.2 | Not changed | 0.6 | White turbid |
| Example 30 | 0.2 | 0.3 | Not changed | 0.4 | Substantially |
| | | | | | not changed |
| Example 31 | 0.2 | 0.2 | Not changed | 1.1 | White turbid |
| Comparative | 0.2 | 1.3 | White | 8.2 | Severely white |
| Example 7 | | | turbid/yellowed | | turbid/yellowed |

1. A resin composition comprising 100 parts by mass of the polymer having an alicyclic structure at least in a part of a repeating structural unit and 0.05 to 5 parts by mass of a hindered amine compound having a carbon atom at a ratio of from 67% by weight to 80% by weight in the molecular structure and having a molecular weight of from 500 to 3500.

2. The resin composition according to claim 1, wherein the solubility of the hindered amine compound in 100 g of hexane at 23° C. is 25 g or more.

3. The resin composition according to claim 1, wherein the solubility of the hindered amine compound in 100 g of hexane at 23° C. is 100 g or more.

4. The resin composition according to claim 1, wherein when the hindered amine compound is heated at 5° C./minute under nitrogen, the 5% by weight reducing temperature at heating of the hindered amine compound is 300° C. or higher.

5. The resin composition according to claim 1, wherein the hindered amine compound is represented by the following General Formula (1): 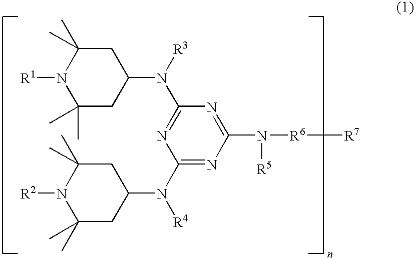
wherein, in Formula (1), n represents 1 or 2,
R¹ and R² may be the same as or different from each other, and each represent a hydrogen atom or a methyl group,
R³, R⁴ and R⁵ may be the same as or different from each other, and each represent a hydrogen atom, an alkyl group having 1 to 24 carbon atoms, the saturated hydrocarbon group having an alicyclic skeleton having 5 to 12 carbon atoms, in which the alicyclic skeleton may have 1 to 3 alkyl substituents having 1 to 4 carbon atoms, a group represented by —R^{A}—Ph(—R^{B})p (wherein R^{A} represents an alkylene group having 1 to 3 carbon atoms, and Ph represents a phenyl group that is unsubstituted or substituted with an alkyl group having 1 to 4 carbon atoms represented as R^{B} and p is an integer of 0 to 3), a substituted alkyl group having 2 to 4 carbon atoms, which has at least one substituent on a carbon atom other than the carbon atom to which a nitrogen atom is directly bonded, in which the substituent is selected from an OH group, an alkoxy group having 1 to 8 carbon atoms, and a dialkylamino group (a plurality of the alkyl groups, may be the same as or different from each other, and are each an alkyl group having 1 to 4 carbon atoms),
R⁶ represents an alkylene group having 1 to 4 carbon atoms, or a single bond, and
R⁷ represents a hydrogen atom,
an aliphatic saturated hydrocarbon group having 1 to 17 carbon atoms,
a saturated hydrocarbon group having an alicyclic skeleton having 5 to 12 carbon atoms, in which the alicyclic skeleton may have 1 to 3 alkyl substituents having 1 to 4 carbon atoms,
a group represented by —R^{7A}-Ph(—R^{7B})p (wherein R^{7A} represents a divalent or trivalent saturated hydrocarbon group having 1 to 3 carbon atoms, and Ph represents a phenyl group that is unsubstituted or substituted with an alkyl group having 1 to 4 carbon atoms represented as R^{7B} and p is an integer of 0 to 3),
a N,N-dialkylamino group represented by —N(R^{7F})(R^{7G}) (wherein R^{7F} and R^{7G} each independently represent an alkyl group having 1 to 18 carbon atoms), or a group represented by —N(R^{7F})— (wherein R^{7F} represents an alkyl group having 1 to 18 carbon atoms, and “—” represents a bond),
a substituted aliphatic saturated hydrocarbon group in which an aliphatic saturated hydrocarbon group has 2 to 4 carbon atoms, which has at least one substituent on a carbon atom other than the carbon atom to which R⁶ is directly bonded, in which the substituent is selected from an OH group, an alkoxy group having 1 to 8 carbon atoms, and a dialkylamino group (a plurality of the alkyl groups, may be the same as or different from each other, and are each an alkyl group having 1 to 4 carbon atoms), or
a group represented by the following formula: 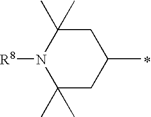
wherein R⁸ represents a hydrogen atom or a methyl group, and * represents a bond.

6. The resin composition according to claim 1, wherein the hindered amine compound is represented by the following General Formula (2): 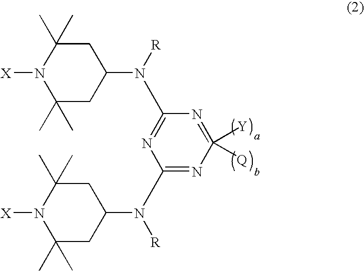
wherein, in Formula (2), a and b are each 0 or 1, and satisfy a+b=1,
R represents an alkyl group having 1 to 24 carbon atoms,
Y is represented by the following Formula: 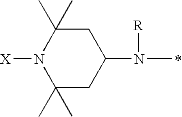
wherein X represents a hydrogen atom or an alkyl group having 1 to 24 carbon atoms, R represents an alkyl group having 1 to 24 carbon atoms, and * represents a bond, and
Q is a group represented by the following Formula: 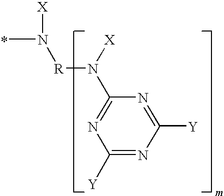
wherein m is 0 or 1, and X and Y are the same as above, R represents, in a case of m=0, an alkyl group having 1 to 24 carbon atoms, or in a case of m=1, an alkylene group having 1 to 24 carbon atoms, * represents a bond, and
a plurality of X, Y, and R, may be the same as or different from each other.

7. The resin composition according to claim 6, wherein X in the above General Formula (2) is a hydrogen atom or a methyl group.

8. The resin composition according to claim 1, wherein the polymer is represented by the following General Formula (3): 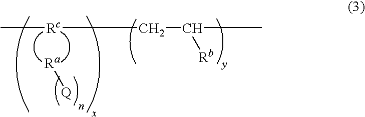
wherein, in Formula (3), x and y each represents a copolymerization ratio, and are each a real number satisfying 0/100≦y/x≦95/5, x and y are based on moles,
n represents a number of a substituent Q, and is a real number satisfying 0≦n≦2,
R^{a} is a 2+n valent group selected from the group consisting of hydrocarbon groups having 2 to 20 carbon atoms,
R^{b} is a hydrogen atom, or a monovalent group selected from the group consisting of hydrocarbon groups having 1 to 10 carbon atoms,
R^{c} is a tetravalent group selected from the group consisting of hydrocarbon groups having 2 to 10 carbon atoms,
Q is COOR^{d} (wherein R^{d} is a hydrogen atom or a monovalent group selected from the group consisting of a hydrocarbon group having 1 to 10 carbon atoms), and
R^{a}, R^{b}, R³, and Q may be each one kind, or a combination of two or more kinds thereof at any ratio.

9. The resin composition according to claim 1, wherein the polymer is a polymer having one or two or more kinds of the structures represented by the following General Formula (4): 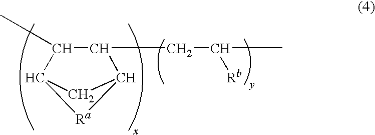
wherein, in Formula (4), R^{a} is a divalent group selected from the group consisting of hydrocarbon groups having 2 to 20 carbon atoms,
R^{b} is a hydrogen atom, or a monovalent group selected from the group consisting of hydrocarbon groups having 1 to 10 carbon atoms,
R^{a} and R^{b} may be each one kind, or a combination of two or more kinds thereof at any ratio, and
x and y each represent a copolymerization ratio, and are each a real number satisfying 5/95≦y/x≦95/5 and x and y are based on moles.

10. The resin composition according to claim 8, wherein the copolymerization ratio y/x of the polymer is 50/505 y/x≦95/5.

11. The resin composition according to claim 8, wherein the polymer is a copolymer of ethylene and a cyclic olefin, and the cyclic olefin is selected from the group consisting of tetracyclo[4.4.0.1^{2,5}.1^{7,10}]-3-dodecene, 1,4-methano-1,4,4a,9a-tetrahydrofluorene, a cyclopentadiene-benzaine adduct, and a cyclopentadiene-acenaphthylene adduct.

12. The resin composition according to claim 8, wherein the polymer is a hydrogenated polymer.

13. The resin composition according to claim 8, wherein the polymer is a vinyl alicyclic hydrocarbon polymer.

14. The resin composition according to claim 1, wherein the content of an iron atom is 5 ppm or less.

15. The resin composition according to claim 1, which further comprises 0.01 to 1 parts by mass of a phosphorus stabilizer.

16. The resin composition according to claim 15, wherein the phosphorus stabilizer has a phosphoric ester structure and a phenol structure in one molecule.

17. The resin composition according to claim 15, wherein the phosphorus stabilizer is represented by the following General Formula (5): 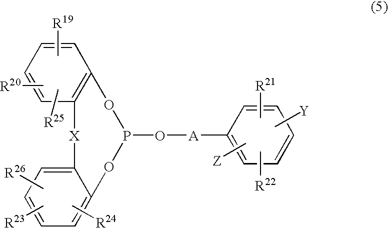
wherein, in General Formula (5), R¹⁹ to R²⁴ each independently represent a hydrogen atom, an alkyl group having 1 to 8 carbon atoms, a cycloalkyl group having 5 to 8 carbon atoms, an alkyl cycloalkyl group having 6 to 12 carbon atoms, an aralkyl group having 7 to 12 carbon atoms, or a phenyl group, and R²⁵ to R²⁶ each independently represent a hydrogen atom or an alkyl group having 1 to 8 carbon atoms,
X represents a single bond, a sulfur atom, or a —CHR²⁷— group (wherein R²⁷ represents a hydrogen atom, an alkyl group having 1 to 8 carbon atoms, or a cycloalkyl group having 5 to 8 carbon atoms),
A represents an alkylene group having 2 to 8 carbon atoms or a *—COR²⁸— group (wherein R²⁸ represents a single bond or an alkylene group having 1 to 8 carbon atoms, and * represents bonding to an oxygen atom side), and
one of Y and Z represents a hydroxyl group, an alkoxy group having 1 to 8 carbon atoms, or an aralkyloxy group having 7 to 12 carbon atoms, and the other represents a hydrogen atom or an alkyl group having 1 to 8 carbon atoms.

18. The resin composition according to claim 15, wherein the phosphorus stabilizer has a saturated alkyl chain structure having 6 or more carbon atoms.

19. The resin composition according to claim 18, wherein the phosphorus stabilizer is represented by the following General Formula (6): 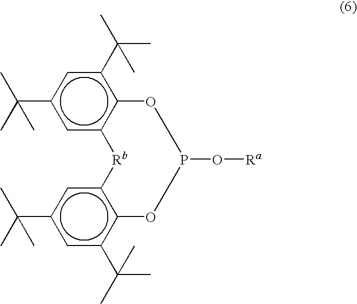
wherein R^{a} represents an alkyl group having 1 to 24 carbon atoms, and R^{b} represents a single bond, a sulfur atom, or a —CHR^{c}— group (wherein R^{c} represents a hydrogen atom, an alkyl group having 1 to 8 carbon atoms, or a cycloalkyl group having 5 to 8 carbon atoms).

20. The resin composition according to claim 1, which further comprises 0.05 to 5 parts by mass of a hydrophilic stabilizer.

21. The resin composition according to claim 1, which further comprises inorganic fine particles having an average particle diameter of 1 nm to 30 nm.

22. The resin composition according to claim 1, which is used in the preparation of a molded product.

23. A piperidine derivative or a salt thereof represented by the following General Formula (20): 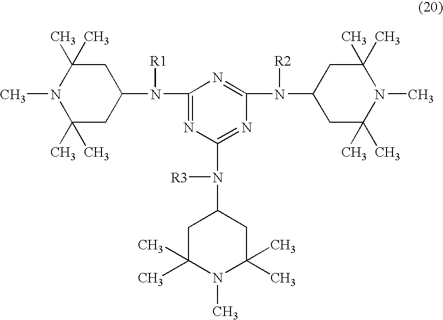
wherein R1 to R3 may be the same as or different from each other, and each represent an alkyl group having 1 to 18 carbon atoms.

24. The piperidine derivative or a salt thereof according to claim 23, wherein R1 to R3 in the above General Formula (20) are all the same.

25. The piperidine derivative or a salt thereof according to claim 24, wherein R1 to R3 in the above General Formula (20) are all an alkyl group having 4 to 12 carbon atoms.

26. The resin composition according to claim 1, which comprises a piperidine derivative or a salt thereof represented by the following general formula (20) as the hindered amine compound 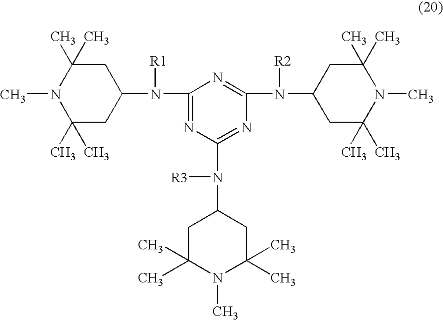
wherein R1 to R3 may be the same as or different from each other, and each represent an alkyl group having 1 to 18 carbon atoms.

27. A molded product obtained from the resin composition according to claim 1.

28. An optical component comprising the molded product according to claim 27.

29. The optical component according to claim 28, which has an optical path difference providing structure.

30. The optical component according to claim 28, which is used for an optical pickup device.

31. The optical component according to claim 30, wherein the optical pickup device is capable of recording or playing back the information on a plurality of the optical information recording media having different substrate thickness, utilizing a plurality of light sources having different wavelength.

32. The optical component according to claim 31, wherein at least one of the light sources has a wavelength of 390 nm to 420 nm.

33. The optical component according to claim 28, wherein at least one portion of the optical component is capable of operating while being supported on an actuator.

34. An optical pickup device, which utilizes the optical component according to claim 28.

35. The optical component according to claim 28, which is used in an optical system having a light source having a wavelength in a range from 300 nm to 450 nm.

36. An outdoor component comprising the molded product according to claim 27.

37. A method for using the resin composition according to claim 1 as a material for an optical component.
